(19) United States
(12) Patent Application Publication
(10) Pub. No.: US 2019/0183938 A1
(43) Pub. Date: Jun. 20, 2019

(54) Provision of a Therapeutically Active Cell Product

(71) Applicant: PDI Pharm Development International AG, Hergiswil (CH)

(72) Inventor: Wojciech Orlowski, Lodz (PL)

(73) Assignee: PDI Pharm Development International AG, Hergiswil (CH)

(21) Appl. No.: 16/328,853

(22) PCT Filed: Aug. 29, 2017
(86) PCT No.: PCT/CH2017/000080

§ 371 Date: Feb. 27, 2019

(30) Foreign Application Priority Data
Aug. 29, 2016 (CH) 1109/16

(51) Int. Cl. A61K 35/28; C12N 5/0775; C12N 15/02; A61P 3/10

(52) U.S. Cl. CPC A61K 35/28; A61P 3/10; C12N 15/02; C12N 5/0663

(57) ABSTRACT

A therapeutically active cell product and an ex vivo method for preparing it from a tissue donation are provided. The method comprises an in vitro immuno-depletion of hematopoietic stem cells and cells of hematopoietic lineage. The therapeutically active cell product obtained by the method comprises a portion of non-hematopoietic stem cells comprising non-hematopoietic progenitor [stem] cells, multipotent stem cells and/or pluripotent stem cells. Further aspects include the cell product for therapeutic use, in particular for the treatment of autoimmune and/or neurological disease.

### TECHNICAL FIELD

The present invention relates to a therapeutically active cell product which comprises non-hematopoietic stem and progenitor cells and it relates to a method of providing the cell product.

### BACKGROUND ART

Adult tissues of human and animal organisms comprise adult stem cells, which are known to self-renew and differentiate into tissue cells and thereby physiologically provide for tissue repair and tissue regeneration. This makes the use of adult stem cells attractive for clinical applications.

Adult stem cells were isolated from diverse tissue sources such as umbilical cord, bone marrow, and adipose tissue. Hematopoietic stem cells isolated from bone marrow have been used for treatment of patients with hematologic malignancies for many years already and today clinical applications of adult stem cell procedures still mainly relate to hematopoietic stem cells.

However, bone marrow not only contains a heterogeneous population of hematopoietic stem cells but also of non-hematopoietic stem cells. Non-hematopoietic stem cells of bone marrow includes e.g. mesenchymal stem cells, tissue specific progenitor stem cells and multi- or pluripotent stem cells which have the capacity to differentiate into various different tissues. For some mesenchymal stem cells it is known that they possess a multipotent differentiation potential.

Meanwhile, several hundred clinical studies of stem cells-based treatment were performed in order to test and document the feasibility and efficacy, as can be derived e.g. from publications in the internet such as in the official database of US National Institutes of Health available on (http://www.clinicaltrial.gov/) or on other sites such as http://neuro.cellthera.org/for-specialists. Many of the clinical trials using non-hematopoietic stem cells are still in progress.

A major difficulty for establishing routine clinical applications using non-hematopoietic stem cell treatments or in particular using mesenchymal stem cells are due to difficulties in obtaining [mesenchymal] stem cell products. Still, no standard method of providing non-hematopoietic stem cells has been established which makes evaluation of the therapeutic effect and comparison between clinical studies difficult.

Several difficulties regarding the provision of mesenchymal stem cells for transplantation have to be overcome: First, mesenchymal stem cells are rare, e.g. in bone marrow they make up a portion of only 0.001 to 0.05% of the cells. Furthermore, there is a lack of consistently expressed surface antigens for the identification and isolation of mesenchymal stem cells on the basis of positive markers as is possible e.g. for hematopoietic stem cells. The International Society for Cellular Therapy (ISCT) published a position paper in 2006 for establishing minimal criteria which are characteristic of MSCs, such as plastic adherence, multipotent differentiation potential in vitro and the presence of CD105, CD73 and CD90 on over 95% of the cells and the essential lack, i.e. expression on less 2% of the cells of a combination of CD45, CD34 and CD14 or CD11b or CD79α or CD 19. However, these criteria suffer from draw backs such as the fact that CD105, CD73 and CD90 are not only expressed on mesenchymal stem cells MSC but are expressed also on many other cell types and that the differentiation potential of mesenchymal stem cells observed in vitro and in vivo is not necessarily the same and may further vary e.g. with the source of the cells or the culture conditions. In fact, numerous obstacles including that mesenchymal stem cells cannot be generally defined by their surface markers (surface antigens), that MSCs produce non homogenous cell populations as a result of in vitro cultivation and that results obtained from the isolation and cultivation of murine mesenchymal stem cells cannot be transferred to human mesenchymal stem cells still hinder the provision of therapeutically active cell products comprising mesenchymal and other stem cells for the treatment of diseases such as autoimmune diseases and/or neurological diseases involving tissue degeneration.

### Disclosure of the Invention

Hence, a general object of the invention is a method to provide a therapeutically active non-hematopoietic stem cell product. The cell product and a method of providing it seek to overcome, alleviate or eliminate above mentioned disadvantages singly or in any combination.

Particular objects of the invention include a method of providing a cell product comprising non-hematopoietic organ-specific progenitor stem cells, pluripotent stem cells and multipotent stem cells and in particular mesenchymal stem cells.

Objects of the invention further include the provision of cell products for medical use as needed after tissue damage or tissue loss due to various reasons including auto-immune and neurological diseases or tissue necrosis due e.g. toxins, infection or trauma. A particular object is to provide a cell product which promotes, improves or enables tissue regeneration in conditions, where therapeutic treatment of tissue damage so far is unsatisfactory. Such objects include providing an improved cell product for treating autoimmune diseases and/or neurological diseases such as e.g. multiple sclerosis and other diseases with progressive loss of some cell and tissue types.

Such objects were solved by the provision of a cell product and a method of preparing it according to the independent claims with further embodiments according to the dependent claims. In order to implement these and still further objects of the invention, which will become more readily apparent as the description proceeds, the following aspects of the invention are manifested as described below.

A first aspect of the invention relates to a method of providing a therapeutically active cell product which is prepared ex vivo from a tissue donation forming an original population of cells. A washed and/or singled cell suspension may be prepared from the original population. The method comprises an in vitro immuno-depletion of hematopoietic stem cells and cells of hematopoietic lineage. The immuno-depletion comprises a depletion of cells expressing at least one selected surface antigen of a group of selected surface antigens. The group of selected surface antigens comprises at least one member of the CD45 surface antigen family, in particular CD45, and at least three surface antigens selected from CD14, CD19, CD34, further members of the CD45 surface antigen family and ICAM-1. The therapeutically active cell product obtained by the method comprises a portion of non-hematopoietic stem cells comprising non-hematopoietic progenitor [stem] cells, multipotent stem cells and/or pluripotent stem cells.

The tissue for the donation can be any tissue comprising non-hematopoietic stem cells. A preferred tissue is bone marrow.

The immuno-depletion comprises an immuno-labeling procedure in which cells expressing one or more of the selected surface antigens are labeled with specific antibodies and a separation procedure for removal of the immuno-labeled cells such as in particular hematopoietic cells from the original cell population.

According to the method of the invention the cell product is prepared ex vivo from an autologous or a heterologous donor tissue such as e.g. bone marrow or another tissue. Medical methods for obtaining donor tissue from a human or animal individual such as for obtaining e.g. bone marrow, peripheral or menstrual blood or tissue probes from umbilical cord, adipose tissue, such as abdominal fat or skin or Wharton's jelly are well established and are not subject of the method according to the invention. Similarly, physical acts of administrating the cell product to a human or an animal are not part of the method of providing the cell product according to the invention.

The cells contained in the donor tissue used for the preparation of the cell product are referred to as the original population of cells. The original population of cells may be suspended yielding an original cell suspension.

In some embodiments, the original cell population obtained from the donated tissue may be fractionated or partially purified prior to the immuno-depletion. Such prior step may efficiently remove particular cell types which are part of a tissue e.g. some blood cells from a tissue highly supplied with blood such as in bone marrow. For example, a washing and filtering step may remove e.g. erythrocytes, cells which are larger than the selected pore size of a filter, cell debris and other components obtained together with the tissue probe. The filtering step may also be applied for singling the cells of the tissue probe and removing tissue lumps.

The term “cell suspension” applies to any cell suspension during the sequence of steps of the in vitro method as apparent from the context. Accordingly, except from the cellular composition of the original cell suspension, the compositions of all of the subsequent cell suspensions differ from the cellular composition of original cell population.

The term hematopoietic cells refers to cells of the hematopoietic system, including hematopoietic stem cells and cells of hematopoietic lineage at various stages during differentiation including e.g. lymphoid or myeloid progenitor cells, erythroblasts and fully differentiated hematopoietic cells, such as e.g. erythrocytes, platelets, macrophages, granulocytes or lymphocytes such as B- and T-cells.

Surprisingly, it has been found that the cell product of the method of the invention is therapeutically active. It comprises non-hematopoietic stem cells and is enriched in some non-hematopoietic stem cells, in particular some cell types expressing surface antigens characteristic of mesenchymal stem cells, due to the removal of hematopoietic cells.

Depending on the use of the cells for some embodiments it may be desired to remove certain cell types completely or essentially completely, thus to deplete e.g. over 90% or in particular over 95% or 99% of a particular selected cell type from the original cell population. In some embodiments, this may be pursued e.g. for a subset of hematopoietic cells e.g. lymphocytes, in particular B- and T-cells and the corresponding progenitor stem cells differentiating to cells mediating the adaptive immune system, or for any cell type known to cause or contribute to tissue damage due to auto immune activity or for removal of cancer or pre-cancerous cells from the cell product.

However, the therapeutic activity of the cell product does not generally require complete or essentially complete depletion of all of the cells expressing one or more of the selected surface antigens or, in particular, of all of the hematopoietic cells. In fact the depletion of the cells expressing at least one of the selected surface antigens, or the “selected cells” for short, may vary depending on the particular selected surface antigen and other factors including the source of the original tissue donation. In some embodiments depletion may range for various selected cell types e.g. from approximately 15% or 20% or 30% or 40% or 50% or 60% or 70% or 80% or 90% to almost all of the selected cells removed from the original suspension, thus a depletion may range from a low or a significant removal of the selected cells to an essentially complete or complete depletion of the selected cells. Accordingly, in some embodiments the cell product may still comprise a portion of hematopoietic cells expressing a particular selected surface antigen that, however, preferably is less than 50% or in particular less than 30% or less than 10%.

By applying an immuno-depletion with several selected surface antigens a particularly efficient removal of target cells simultaneously expressing two or more of the selected surface antigens is achieved. The immuno-depletion in the method according to the invention benefits from this effect because it requires a group of at least four selected antibodies for the immuno-depletion.

The portion of cells comprising non-hematopoietic progenitor [stem] cells, non-hematopoietic multipotent stem cells and non-hematopoietic pluripotent stem cells, which is contained in the cell product or enriched in the cell product compared to the original population of cells due to the removal of in particular hematopoietic cells, is for short referred to in this text as non-hematopoietic progenitor and stem cells or even shorter as non-hematopoietic stem cells. It is defined as a portion of cells which are able to self-renew and which are not committed and/or not irreversibly committed to a cell type for or within the hematopoietic lineage. Thus, the portion of non-hematopoietic stem cells includes e.g. non-hematopoietic stem cells with a very broad differentiation potential, even pluripotent stem cells, very naive stem cells, cells called very small embryonic like stem cells (VSELs), multipotent stem cells, and in particular also mesenchymal stem cells which are known to provide a therapeutic benefit in tissue regeneration. The portion of non-hematopoietic stem cells also includes non-hematopoietic progenitor [stem] cells. Progenitor cells generally exhibit a more limited capacity to self-renew than other stem cells and usually they are unipotent, i.e. determined for differentiation into one particular somatic cell type. Progenitor cells are sometimes also called determined stem cells. However, progenitors are not necessarily irreversibly committed to the determined cell type and depending on the cellular environment and/or influence of trophic factors, progenitor cells may transdifferentiate into another cell type. The portion of non-hematopoietic stem cells also includes multipotent adult progenitor cells (MAPCs). The terminology in stem cell biology for some cell types is still evolving and sometimes cells with essentially the same level of sternness and/or differentiation potential are termed differently and, vice versa, some cells which differ in the differentiation potential and/or the level of sternness are referred to by the same name. As indicated above, in the scope of the present invention the term stem cells encompasses progenitor cells (which may be indicated by the notation “progenitor [stem] cells”).

As known in the art, cells express on their surface a number of antigens which are characteristically present or absent depending on the particular (stem) cell type. Therefore, the analysis of surface antigens, which are also called just antigens or markers, allows characterizing different cell types according to their surface antigen profile. The cellular composition of the original and subsequent cell suspensions and of the cell product may be described by counting the number of cells expressing particular cell surface antigens, in particular in comparison to the total cell number. Many of the surface antigens belong to the CD (cluster of differentiation) surface antigens.

As conventional in the art, in this text cell types are specified by their physiological role such as e.g. hematopoietic cells, lymphocytes, mesenchymal stem cells and/or by their antigen profile, indicating whether one or more surface antigen is present or absent on the surface such as e.g. CD34-positives (CD34⁺), or CD14-negatives (CD14⁻).

It is well-known in the art that surface antigen expression is a dynamic cellular process depending on e.g. developmental stage, cellular health, tissue environment, age and other factors. Thus, a particular cell changes its surface antigen profile e.g. along its differentiation pathway and depends e.g. on environmental factors such as tissue or cellular factors on its stage of health or age. Therefore, such circumstances, have to be considered in the choice of surface antigens serving as a selected surface antigen for the immuno-depletion of the method.

For example, in the differentiation process of the hematopoietic system, hematopoietic stem and progenitor cells change their surface antigen profile as a function of time and in particular also of their environment when they migrate from the bone marrow into the peripheral blood system, (for more information e.g. ZYDOWICZ, B. MAZUR, “Cells Immunophenotype in Normal Hematopoiesis”, Postepy Biologii Komoriki Tom 35, 2008, Suplement Nr. 24 35-44 http://pbkom.eu/sites/default/files/artykulydo2012/35_s24_35.pdf) or e.g. Attar A., “Changes in the Cell Surface Markers During Normal Hematopoiesis: A Guide to Cell Isolation.”Global Journal of Hematology and Blood Transfusion, 1, 20-28, 2014 or e.g. van Lochem E. G., “Immunophenotypic differentiation patterns of normal hematopoiesis in human bone marrow: reference patterns for age-related changes and disease-induced shifts.” Cytometry Part B (Clinical Cytometry) 60B:1-13, 2004).

Such variability may be useful to take into account when selecting surface antigens for immuno-depletion procedures of the method for obtaining cell products for different therapeutic applications. For example, for the correlation of the surface antigen profile with a particular cell type, besides general knowledge in the art, particular circumstances regarding the source of the tissue donation are advantageously taken into account. Generally according to currently available information the following surface markers are included as characteristic surface antigens for non-hematopoietic stem and progenitor cells: CD105, SSEA-4, CD166, CD146, CD44, CD71, CD90, CD73, CD106, CD117, CD133, co-expression of both CD34 and CD 133.

CD133, SSEA4 and CD90 are included as relevant antigens in particular for pluripotent stem cells.

Some of these non-hematopoietic surface antigens which are generally expressed on non-hematopoietic cells are also expressed on some hematopoietic stem or precursor cell types, in particular: CD34, CD117, CD133.

CD14, CD19, CD34, CD45 family and ICAM-1 belong to surface antigens which are expressed on cells of the hematopoietic system.

The CD14 surface antigen (a lipopolysaccharide receptor) is primarily expressed on hematopoietic cells such as on monocytes including macrophages and dendritic cells as well as on neutrophilic granulocytes of the innate immune system. CD14 is also expressed on the surface of some cancer cells such as in myelomonocytic leukemia and histiocytic sarcoma and other forms of cancer as can be derived from the internet e.g. on http://www.cancerindex.org/geneweb/CD14.htm. CD14 is generally not expressed on mesenchymal stem cells.

The CD19 surface antigen is associated with the antigen receptor of B lymphocytes and is present on B cells from very early cells in the B-lineage on during maturation until mature stage of B cells and plasma cells.

CD34 is known to be expressed on hemangioblasts which exist in adult tissue and can differentiate both into hematopoietic and endothelial cells.

The CD34 surface antigen (a glycosylated transmembrane protein) is primarily expressed on hematopoietic stem cells. In particular it is expressed on early hematopoietic cells and cells of vascular-associated tissue. It is normally found in early hematopoietic and vascular-associated tissue, in the umbilical cord and in the bone marrow as marker of hematopoietic stem cells. However, CD34 is also expressed on a subset of mesenchymal stem cells, on endothelial progenitor cells, endothelial cells of blood vessels but not lymphatics (except pleural lymphatics).

Some embodiments of the invention which are considered advantageous remove part but not all of the CD34 expressing cells from the cell product.

The selected surface antigens CD14, CD19, CD45 family and ICAM-1 are generally expressed on hematopoietic cells.

The ICAM-1 surface antigen, also termed CD54, is generally expressed on macrophages and lymphocytes and their stem and precursor cells and also on endothelial cells.

The CD45 surface antigen family (various forms of the protein tyrosine phosphatase receptor C—PTPRC; formerly known as LCA—leucocyte common antigen) is expressed on almost all of the hematopoietic cells except for erythrocytes. Monoclonal anti CD45 antibodies have been routinely used for the identification of leucocytes.

Depending on the cell type various splice and glycosylation variants of CD45, e.g. CD45RA, CD45RB, CD45RC, CD45RAB, CD45RAC, CD45RBC, CD45RO, CD45R(ABC) are expressed on the cell surface. For example, CD45RA is present on naive T-cells, CD45RO is expressed on activated T-cells and T-memory cells. CD45R is expressed on B-cells and their precursors, on a sub-group of dendritic cells and other antigen-presenting cells. The CD45 surface antigen family is generally not expressed on mesenchymal stem cells.

The group of selected surface antigens includes at least one surface antigen of the CD45 family, in particular it includes CD45. In further particular embodiments, the group of selected surface antigens includes at least two surface antigens of the CD45 family. In particular embodiments it includes CD45 and one or more further surface antigens of the CD45 antigen family such as CD45 and CD45RA or CD45 and CD45RO, or CD45, CD45RA and CD45RO or other combinations of members of the CD45 antigen family.

In some embodiments the group of selected surface antigens includes CD14, CD34, and at least one member of the CD45 family.

In some embodiments the group of selected surface antigens includes CD14, CD34, CD45 and at least one further member of the CD45 family, in particular CD45 and e.g. CD45RA or CD45RO. The use of this group of selected surface antigens in the method results in particularly efficient depletion of CD34 cells co-expressing CD45RA or CD45RO.

The group of selected antigens may also comprise further additional antigens other than those mentioned above as indicative of hematopoietic stem cells.

For example such additional surface antigens encompass antigens which are characteristic for various cell types such as older cells, highly differentiated cells, cancer cells or pre-cancerous cells prone to malignant transformation. The group of selected antigens may include in particular further antigens which are not expressed on non-hematopoietic stem cells and/or are not expressed on cells which are known to be beneficial for tissue regeneration, e.g. cells which secrete factors like differentiation factors, growth factors or factors which promote stem cells to differentiate and replace lost cells at a site of tissue damage.

For the selection of further surface antigens to be included in the group of selected surface antigens, the skilled person may take into account the tissue source and/or particular use of the cell product and in particular may apply one or more of the following criteria
additional surface antigens, which are characteristic for one or more cell type of hematopoietic stem cells and/or cells of hematopoietic lineage, including e.g. at least one of CD2, CD3, CD10, CD11b, CD15 (SSEA-1), CD16, CD44, CD56, CD123, CD235a, CD326, CD49f;
additional surface antigens, which are absent or essentially absent on mesenchymal stem cells or on cells which have been reported to promote tissue regeneration, e.g. at least one of CD11a/LFA-1, CD31, CD80, CD86, CD40 and CD144.
surface antigens, which are present on cancer cells or precancerous cells or on cells promoting the transformation of stem cells, in particular at least one of CD9, CD15, CD20, CD24, CD31, CD38, CD44, CD117, CD146, CD166, CD171, CD184, CD324, CD325, CD326, CD338, ERb2 or HER2/neu.

Besides taking into account the dynamics of cell surface antigen expression depending e.g. on the state and source of cells, the skilled person also is aware that stem cell biology is an active area of research and will consider pertinent current sources of information e.g. in the internet or printed literature as applicable.

A small exemplary collection of references is listed below:
Attar A.
   Changes in the Cell Surface Markers During Normal Hematopoiesis: A Guide to Cell Isolation. Global Journal of Hematology and Blood Transfusion, 1, 20-28, 2014
Brzozowski A., Dmoszyńska A.
   Bone marrow-derived Endothelial Progenitor Cells: the biology, functions and clinical applications.
   Acta Haematologica Polonica, 35, 177-187, 2004
Calloni R., Elvira Alicia Aparicio Cordero E. A. A., Pegas Henriques J. A., Bonatto D.
   Reviewing and updating the major molecular markers for stem cells.
   STEM CELLS AND DEVELOPMENT, Volume 22, Number 9, 1455-1476, 2013
Jacobs S. A., Roobrouck V. D., Verfaillie C. M., Van Gool S. W.
   Immunological characteristics of human mesenchymal stem cells and multipotent adult progenitor cells.
   Immunology and Cell Biology, 91, 32-39, 2013
Lin Ch. S., Ning H., Lin G., Lue T. F.
   Is CD34 truly a negative marker for Mesenchymal Stem Cells?
   Cytotherapy, 14 (10), 2012
Lin Ch. S., Xin Z. Ch., Dai J., Lue T. F.
   Commonly used Mesenchymal Stem Cell markers and tracking labels: limitations and challenges. Histol Histopathol., 28(9), 1109-1116, 2013
van Lochem E. G.,
   Immunophenotypic differentiation patterns of normal hematopoiesis in human bone marrow: reference patterns for age-related changes and disease-induced shifts.
   Cytometry Part B (Clinical Cytometry) 60B: 1-13, 2004.
Mafi P., Hindocha S., Mafi R., Griffin M., Khan W. S.
   Adult Mesenchymal Stem Cells and cell surface characterization—a systematic review of the literature.
   The Open Orthopaedics Journal, 5, (Suppl 2-M4) 253-260, 2011
Maleki M., Ghanbarvand F., Behvarz M. R., Ejtemaei M., Ghadirkhomi E.
   Comparison of Mesenchymal Stem Cell markers in multiple human adult stem cells.
   International Journal of Stem Cells, Vol. 7, No. 2, 118-126, 2014
Herrmann M., Binder A., Menzel U., Zeiter S., Alini M., Verrier S.
   CD34/CD133 enriched bone marrow progenitor cells promote neovascularization of tissue engineered constructs in vivo.
   Stem Cell Research, 13, 465-477, 2014
Murphy M. B., Moncivais K., Caplan A. I.
   Mesenchymal stem cells: environmentally responsive therapeutics for regenerative medicine.
   Experimental & Molecular Medicine, 45, 2013
Pojda Z., Machaj E., Kurzyk A., Mazur S., Dębski T., Gilewicz J., Wysocki J.
   Mesenchymal Stem Cells.
   Postępy Biochemii, 59 (2), 187-197, 2013
Shi C.
   Recent progress toward understanding the physiological function of bone marrow mesenchymal stem cells.
   Immunology, 136, 133-138, 2012
de Vasconcellos Machado C., da Silva Telles P. D., Nascimento I. L. O.
   Immunological characteristics of mesenchymal stem cells.
   Rev Bras Hematol Hemoter.,35(1), 62-67, 2013
Zou Z., Zhang Y., Hao L., Wang F., Liu D., Su Y., Sun H.
   More insight into mesenchymal stem cells and their effects inside the body.
   Expert Opin. Biol. Ther., 10(2), 215-230, 2010
Zydowicz G., Mazur B.
   Cells immunophenotype in normal hematopoiesis. Postępy Biologii Komórki, tom 35, suplement nr 24, 34-44, 2008

In an embodiment of the inventive method the immuno-depletion comprises an immuno-labeling procedure labeling cells expressing one or more of the selected surface antigens with specific antibodies comprising a tag

wherein the tag may be conjugated to the antibodies prior to a specific binding of the antibodies to the surface antigens or the tag may be conjugated to the antibody after the specific binding of the antibodies to the surface antigens during the immuno-labeling procedure,

wherein the tag in particular is a magnetic bead or a fluorescent tag;

and wherein the immuno-depletion comprises a separation procedure for removal of the cells labeled with antibodies comprising a tag from the suspension in step separate from the labeling procedure or in a combined step.

Depletion methods and in particular immuno-depletion methods for in vitro removal of specific cells from a cell population are well known in the art and comprise a labeling procedure, in which the cells to be removed are specifically labeled and a separation procedure for removing the labeled cells from the unlabeled cells in a combined step or in separate steps. The labeling and separation procedure may be performed combined or in separate steps, in particular with removal of excess labeling reagents such as antibodies, tag-conjugated reagents such as tag conjugated secondary antibodies etc. prior to the separation procedures in which the labeled cells are separated from the unlabeled cells. The unlabeled cells remaining in the resulting cell population after depletion of the labeled cells are the desired product according to the present invention. In some embodiments the labeling and separation procedures may be combined e.g. without separation of unreacted labeling reagents prior to separation.

Immuno-depletion methods comprise an immuno-labeling procedure with antibodies comprising a tag for specific binding to cell surface antigens at corresponding antigen binding sites. The tags, e.g. fluorescent compounds or magnetic beads, may be attached to the antibodies prior to the specific binding of the antibodies to the surface antigen or after the specific binding as a part of the immuno-labeling procedure. In the separation procedure the immuno-labeled cells are removed. Methods of immuno-depletion, immuno-labeling and separation techniques such as MACS (magnetic cell sorting or separation) and FACS (fluorescent cell sorting or separation) and others are well known in the art. Advantageously, the cell product of these methods consists of the cells which have not been previously bound and subsequently eluted from antibodies or tagged antibodies or columns.

In some embodiments of the method the immuno-labeling is an immuno-magnetic labeling procedure with a magnetic particle as tag and using a magnetic separation device for the separation procedure in order to deplete the immuno-magnetically labeled cells.

In some embodiments of the method, the immuno-labeling is a direct immuno-labeling procedure with surface antigen specific antibodies which are conjugated to tags prior to the labeling procedure. For such direct immuno-labeling the cell suspension is incubated with one or with several tag-conjugated surface antigen specific antibodies. The tag-conjugated antibodies such as antibodies conjugated to a magnetic particle or to a fluorescent tag may be available commercially or may be obtained according to methods well known in the art. Such direct immuno-labeling of cells with tag-conjugated antibodies may comprise one or more than one incubation step with one or several tag-conjugated antibodies present during incubation of the cell suspension.

In some embodiments, the method comprises an indirect immuno-labeling procedure with the use of surface antigen specific antibodies which are conjugated to tags during the labeling procedure. In an exemplary embodiment of indirect labeling, in a first step the cell population is incubated with one or in particular with several surface antigen specific primary antibodies. Excess unbound primary antibodies are preferably removed by centrifugation and re-suspension of the cells after the first incubation step. Subsequently, in a second step the cell population is incubated with tag-conjugated secondary antibodies or with another tag-conjugated reagent that specifically binds to primary antibodies. In the first step e.g. biotinylated primary antibodies may be used and in the second step e.g. streptavidin coated tags may be used. In some embodiments e.g. tag-conjugated anti-biotin antibodies are used. The tag conjugated to secondary antibodies or to another reagent may e.g. be a magnetic particle such as an iron-dextran bead or a fluorescent tag. Excess unbound secondary antibodies are preferably removed by centrifugation and re-suspension of the cells after the second step. The immuno-labeling optionally comprises additional steps before and/or after the first and the second step. In some embodiments the number of incubations in the first and the second step combined is limited to a total of up to two or up to three or up to four or up to five incubations of the cell suspension with primary and/or secondary antibodies.

The inventive method may comprise more than one direct and/or indirect immuno-labeling steps such as up to two, up to three or up to four steps. In some of these and other embodiments the method may also comprise mixed immuno-labeling, i.e. direct labeling and one step of an indirect labeling combined, such as by using an antibody cocktail comprising besides one or more tag-conjugated antibodies also one or more primary unconjugated antibodies and/or tag-conjugated secondary antibodies.

Surprisingly, the portion of the desired non-hematopoietic stem and progenitor cells in the cell product may be even increased by limiting the degree of depletion of some selected cells expressing a selected surface antigen.

Limiting the degree of depletion in this text is also referred to as depletion under limiting conditions. In according embodiments of the method, limiting conditions are chosen such that cells expressing a comparatively low total number of one or more of the selected surface antigens per cell are essentially not depleted or depleted to a lower degree compared to cells expressing a comparatively large number of selected surface antigens per cell which are depleted to a higher degree or depleted completely or essentially completely. A comparatively small number of surface antigens per cell may be present e.g. on the surface of small cells, because small cells due to their small size generally express lower numbers of surface antigens per cell compared to larger cells. Also, surface antigen expression varies not only qualitatively i.e. with respect to the type of surface antigen, but also quantitatively; i.e. the number of surface antigens expressed per cell may vary with the cell type, in particular along its path of differentiation. Furthermore, a comparatively low number of selected surface antigens may be present on cells expressing a moderate number of only one of the selected surface antigens compared to cells expressing moderate levels of two or more of the selected surface antigens. The cell product obtained by depletion under limiting conditions in particular benefits from the advantageous effect that stem cells and early progenitors which are often smaller and/or express fewer surface antigens per cell than cells further down the differentiation pathway are depleted less efficiently.

Depletion under limiting conditions favoring the depletion of cells from the original population of cells which have a higher number of selected surface-antigen-specific antibodies bound per cell compared to cells which have a lower number of such selected antibodies bound per cell may in particular be achieved by selecting one of the following conditions or by selecting a combination of more than one of the following conditions:
in the direct immuno-labeling procedure limiting incubation conditions are allowing for only a partial saturation of the antigenic binding sites of the selected surface antigens on the cells by the tag-conjugated antibodies;
in the first step of the indirect immuno-labeling procedure limiting incubation conditions are allowing for only a partial saturation of the antigenic binding sites of the selected surface antigens on the cells by the primary antibodies;
in the second step of the indirect immuno-labeling procedure limiting incubation conditions are allowing for only a partial saturation of the antigenic binding sites of the primary antibodies by the tag-conjugated secondary antibodies;
in the first step of the indirect immuno-labeling standard incubation conditions are applied, in particular the incubation conditions are adjusted to allow for a maximized saturation of the selected surface antigenic binding sites while minimizing unspecific binding of the primary antibodies to the cells, wherein in the second step limiting incubation conditions are allowing for only a partial saturation of the antigenic binding sites on the primary antibodies by the tag-conjugated secondary antibodies;
in the first step of the indirect immuno-labeling limiting incubation conditions are allowing for only a partial saturation of the selected surface antigenic binding sites on the cells, wherein in the second step standard incubation conditions are applied, in particular the incubation conditions are adjusted to allow for a maximized saturation of the antigenic binding sites on the primary antibodies, in particular of the biotinylated primary antibodies, by the conjugated tag such as in particular tag-conjugated secondary antibodies or in particular anti-biotin secondary antibodies or in particular streptavidin-conjugated tags like streptavidin-coated magnetic particles, while minimizing unspecific binding of conjugated tags to the primary antibodies or to the cell surface;
in the separation procedure cells which are labeled with two or more tags, in particular with at least three or four or more than four tags, are removed from the original cell population whereas cells comprising fewer tags remain in the original cell population, and wherein in particular the tags are magnetic particles.

Standard incubation conditions may refer to conditions which comply with the specifications by the manufacturer of the antibodies or they may be determined in that the binding probability and/or the contact efficiency and/or the binding strength are optimized for maximal saturation with specifically bound antibodies to corresponding antigenic binding sites of selected surface antigens while keeping non-specific binding of antibodies to cells lacking the specific selected surface antigen at a reasonably low level.

Variable degrees of depletion as well as degrees of enrichment may be expressed quantitatively. This text includes the following quantitative terms and definitions for the degree of depletion:

The degree of relative depletion is defined as the ratio of the portion of a particular cell type among the total number of cells in the cell product over the portion of that particular cell type among the total number of cells in the original cell population. A relative depletion is indicated by a value smaller than 1. For example if the portion of a particular cell type in the cell product is 20% and the portion of this particular cell type in the original cell population is 80%, then the relative depletion is 0.25. The relative factor of depletion is defined as the reciprocal of the relative depletion, i.e. a factor of 4 in the above example.

Degrees of depletion may refer to a particular cell type which is in particular is specified by the physiological role (e.g. hematopoietic cells) or by its marker (surface antigen) profile as indicated by the presence or absence of characteristic surface antigens on the cell surface.

Relative enrichment and the relative factor of enrichment are defined as above by the same ratio, which however results in a value above 1 indicating that the portion of a particular cell type in the cell product has increased in comparison to the portion in the original cell population.

Absolute depletion is often expressed as the percentage portion of cells removed from the original cell population, i.e. if 100% are removed than depletion is complete.

The degree of absolute depletion is defined as the ratio of the number of cells of a particular type in the cell product over the number of cells of this cell type in the original population of cells.

The value of the degree of absolute depletion must always be below 1. As an example, a depletion of 80% of the cells indicates that 80% of the cells of a particular cell type present in the original population of cells were removed and 20% are recovered in the cell product resulting in a degree of absolute depletion with a value of 0.2. The absolute factor of depletion is the reciprocal of the absolute depletion and in the above example indicates a 5-fold absolute depletion of this particular cell type.

The depletion procedure cannot increase the absolute number of cells in the product compared to the original cell population. Accordingly, an absolute enrichment is not feasible.

The degree of depletion is influenced by both the efficiency of the labeling procedure and the efficiency of the separation procedure. The efficiency of the labeling procedure is defined as the percentage portion of the number of cells expressing at least one of the selected surface antigens which is labeled with respect to the maximal number of cells expressing the selected surface antigen that can be labeled under optimal conditions, i.e. conditions which avoid significant unspecific labeling as determined by titration or by manufacture's specifications. The efficiency of the separation procedure is defined as the percentage portion of the labeled cells being removed with respect to the maximal number of labeled cells that are present.

Immuno-depletion procedures known in the art may allow absolute depletion of a particular cell type from a mixed population of cells which may exceed 90% or 95% and approach 100% when conditions for saturation of antigenic binding sites in the labeling procedure and conditions for removal of labeled cells are optimized according to standard laboratory techniques. There are also commercially available sets of depletion equipment, reagents and protocols allowing for essentially complete depletion of cells expressing a selected surface antigen or several selected surface antigens.

Surprisingly, it was found, that particularly favorable compositions of the cell population in the cell product are obtainable by deliberately adjusting the conditions of the method to limit the degree of depletion such that weakly labeled cells remain in the cell product whereas strongly labeled cells are removed.

As described above, physiological reasons for weak labelling of cells include in particular cells which exhibit only a small number of one or more of the selected surface antigens in the cell product, e.g. due to their small size or due to low expression of a selected surface antigen.

As shown by FACS analysis of the cell product obtained in particular under conditions limiting the degree of depletion, the cell product may exhibit surprisingly large portions of cells expressing a marker of the CD45 family even in embodiments using CD45 and at least one further member of the CD45 antigen family. Further analysis of the portion of CD45 positive cells in the cell product revealed that it comprises a large number of granulocytes. Exemplary ranges of the percentage ratios of portions of cells expressing the indicated surface antigens in the cell product vs. in the original population of the method according to the invention, including embodiments performed under limiting conditions of the immuno-depletion, are shown in Table A below:

TABLE A

| | |
|---|---|
| | Percentage Ratio of Portions of |
| Surface | Positives in Cell Product vs. |
| Antigen | Original Population (C/A × 100) |

| (CD) | general | preferred |
|---|---|---|
| 117 | ≤2-30 | ≤5 ± 2 |
| 34/133 | ≥10-50 | ≥35 ± 5 |
| 34 | 2-30 | 5-20 ± 2 |
| 14 | ≤5-25 | ≤12 ± 2 |
| 19 | ≤1-20 | ≤9 ± 2 |
| ICAM-1 | ≤1-20 | ≤2 ± 1 |
| 34/45 | ≤1-25 | ≤5 ± 2 |
| 45 | ≤0-45 | ≤10 ± 5 |
| Family | | |
| 45/45RA | ≤0-10 | ≤2 ± 2 |
| 45RA | ≤0-15 | ≤2 ± 2 |
| 11B | ≤70 | ≤25-30 ± 5 |
| 15 | ≤65-70 ± 5 | ≤40 ± 5 |
| 44 | ≤30 | 7-25 |

Advantageously the method yields a cell product with a portion of stem cells which is sufficient to provide a large enough number of stem cells for direct administration to a patient, e.g. systemic administration by intravenous injection, to effect therapeutic activity and benefit the patient receiving it. Thus, the method of the invention yields a cell product ready for transfer to a patient directly derived from the original population of cells with non-substantial manipulations only, and in particular without in vitro cultivation for amplification of the cell number prior to administration. Such administration of the cell product without in vitro amplification avoids non-physiological cell developments known to occur during in vitro cultivation. This is a relevant advantage in view of the problems known to be associated with the negative influence on the therapeutic quality of e.g. positively selected mesenchymal stem cells by in vitro cultivation as used in prior art procedures. In comparison, the portion of non-hematopoietic stem and progenitor cells, in particular mesenchymal stem cells in the cell product according to the second aspect of the invention are of an improved, more natural physiological quality. The therapeutically active cell product is more similar to the tissue source regarding e.g. differentiation stage and cellular environment of the portion of non-hematopoietic stem cells. Furthermore, it exhibits a superb viability of the cells, in a way which was not achievable in the prior art with other methods of preparation of stem cell products, in particular prior art mesenchymal stem cell products.

Nevertheless, if considered beneficial for particular patients, the cell product may also be subjected to in vitro cultivation for increasing the cell number for particular therapeutic applications, in particular embodiments under culture conditions which minimize differentiation of the cells.

Thus, in embodiments of the method of the invention only a short time of few hours is required for performing all of the ex vivo method manipulations between the receipt of a preferably autologous tissue donation and obtaining the final therapeutically active cell product ready for therapeutic administration, which preferably does not exceed 10 or 8 or 6 or 5 hours. In particular, in embodiments of the method apart from an optional washing and filtering step prior to the immuno-depletion no further and in particular no substantial ex vivo manipulations such as gradient centrifugation and/or in particular in vitro cultivation are performed. This results in a method of providing a therapeutically active cell product which is much faster than standard procedures in the art, which in addition to washing and positive or negative selection of cells expressing particular surface antigens usually also comprise time consuming and/or substantial ex vivo manipulations such as purification by gradient centrifugation and in particular in vitro culturing for amplification of desired stem cells.

The above mentioned times for performing all or the ex vivo manipulations does not include sterility tests on a sample of the final cell product which depending on clinical regulations may be required before a cell product is allowed to be administered to a patient.

Thus, the method provides for a way of providing an improved therapeutically active cell product comprising non-hematopoietic stem cells and in particular mesenchymal stem cells.

The above described and further embodiments of the method of providing the therapeutically active cell product are described further in the detailed description and in the example section.

A second aspect of the invention relates to the cell product obtainable by in vitro depletion of hematopoietic cells, and in particular it relates to the cell product obtainable by in vitro depletion of hematopoietic cells according to the method of the first aspect of the invention. The cell product is characterized inter alia by a large diversity of the cell types present in the cell population of the therapeutically active cell product comprising non-hematopoietic stem cells including pluripotent and multipotent stem cells, progenitor [stem] cells and in particular mesenchymal stem cells, by the high viability of the cells and by its therapeutic activity.

Thus, the cell product is not a product of purified non-hematopoietic stem and progenitor cells. Rather, the cell product is a heterogeneous cell population comprising many different cell types. It has a particularly favorable composition and cellular environment which—apart from the depletion of hematopoietic stem and lineage cells closely resembles the physiological composition and cellular environment of the original population of cells in the donated tissue. The cell product has improved therapeutic activity for tissue regeneration compared to cell products obtained by methods common in the art based e.g. on physical separation techniques such as plastic adherence or gradient centrifugation or positive immuno-selection for the provision of a highly selected group of e.g. mesenchymal stem cells for therapeutic applications followed by in vitro culture.

The maintenance of the cellular composition, cellular environment and physiological state of the cells by application of only non-substantial ex vivo manipulations to the cell population during the depletion of hematopoietic cells and in particular the absence of in vivo culturing appears to confer distinct qualities and characteristic advantages for the therapeutic effect of the product according to the second aspect of the invention: Indeed, tissue regeneration according to current understanding is stimulated by important signaling between stem cells and progenitor cells at various stages of commitment for differentiation and other differentiated cells of different types, e.g. including also differentiated cells which secrete growth or differentiation factors. The diversity of cell types in the population of the cell product, which is much larger than in positively selected stem cell products, is regarded as a reason for its surprising therapeutic activity. In some embodiments, the cell product comprises cells which cross the blood brain barrier or cells which secrete factors which cross the blood brain barrier or cells which promote physiological stem cells of the patient receiving the cell product to cross the blood brain barrier indirectly via secreted factors or via direct cellular interactions between transferred cells of the cell product and cells of the patient and thereby the cell product effects repair of damaged tissue in the brain.

In some of these and other embodiments the cell product depleted of hematopoietic cells is particularly efficiently depleted of cells mediating the adaptive immune system, in particular of B cells and of T cells and their corresponding precursor stem and progenitor [stem] cells.

Depletion methods are negative selection procedures which are known to be particularly gentle procedures to the cells which are subjected to the depletion reagents and manipulations. Accordingly, a further advantage of the cell product obtainable by negative selection of non-hematopoietic cells as performed e.g. by the immuno-depletion method according to the method according to the invention is that after the depletion of the hematopoietic cells, those cells which remain and are collected as the cell product for therapeutic administration have not or not significantly been stressed by separation reagents such as antibodies, magnetic tags or fluorescent tags or physical treatments such as plastic adherence. At least most of the cells which are significantly touched by binding to immuno-labelling reagents such as antibodies and other reagents are removed from the cell suspension while the not significantly touched or untouched cells remain in the cell product and the desired progenitor and stem cells exhibit very good viability and healthy physiological activities.

The cell product according to the second aspect and in particular obtainable according to the method of the first aspect of the invention retains at least a significant portion of the non-hematopoietic pluripotent and multipotent stem cells and organ specific progenitor [stem] cells and in particular mesenchymal stem cells which were initially present in the original population of cells of the donated tissue probe and it is therapeutically active.

It is known that e.g. in bone marrow these stem and progenitor cells are present only in very low numbers. For example mesenchymal stem cells are present in bone marrow as a portion in the range of approximately 0.001 to 0.05% of the total number of cells. It is an important property of the cell product depleted of hematopoietic cells according to the second aspect of the invention that it comprises a significant portion of non-hematopoietic stem cells in a viable and therapeutically active state.

In fact, in embodiments of the cell product according to the second aspect of the invention, the cells expressing some of the surface antigens indicative of desired stem cells, i.e. non-hematopoietic pluripotent and multipotent stem and progenitor [stem] cells, in particular mesenchymal stem cells as measured by cytometric analysis constitute a portion in the cell product which is similar to the portion in the original population or which is increased in the cell product. An increased portion of non-hematopoietic stem cells corresponds to an enrichment of the desired stem cells,as defined above. Thus, the number of the desired stem cells in such cell product constitutes a larger portion of the total number of cells in the cell product than the portion which these stem cells constituted in the original population of cells from which the product was derived.

For example in some embodiments of the cell product obtainable by in vitro depletion of hematopoietic cells from a tissue probe according to the second aspect of the invention and in particular in some embodiments obtainable according to the method of the invention, the portions of cells expressing one or more surface antigen indicative of pluripotent stem cells such as cells expressing SSEA-4 or CD90 or CD133 or cells co-expressing CD34/CD133 amount to at least 0.01% to 1% of the total cell number, in particular at least 0.03% or at least 0.1% or at least 0.3% or at least 1% as measured by cytometric analysis.

In further of these and other embodiments of the cell product obtainable by in vitro depletion of hematopoietic cells according to the second aspect and in particular obtainable according to the method of the invention, portions of cells expressing one or more of the surface antigens mentioned above and or one or more of the following surface antigens indicative of multipotent and progenitor stem cells such as CD90, CD133, cells co-expressing CD34/00133, 0044, CD71, CD73, CD105, CD106, 00117, CD146, CD166 or CD34 amount to at least 0.01% to 1%, in particular at least 0.03% or at least 0.1% or at least 0.3% or at least 1% as measured by cytometric analysis.

Furthermore, in some of these and further embodiments of the cell product according to the second aspect of the invention, in particular obtainable according to the method of the first aspect, the portions of cells expressing CD34 or a surface antigen of the CD45 surface antigen family does not exceed 20%, or in particular does not exceed 15%, 10% or 6% or 4% or 2%.

Furthermore, in some of these and further embodiments of the cell product according to the second aspect of the invention by in vitro depletion of hematopoietic cells in particular obtainable according to the method of the first aspect, the portions of cells expressing one of the surface antigens CD14, CD19, ICAM-1 or co-expressing CD45/CD34 does not exceed 5%, in particular not 2% or 1% or 0.5%.

Furthermore, in some of these and further embodiments of the cell product according to the second aspect of the invention by in vitro depletion of hematopoietic cells in particular obtainable according to the method of the first aspect the portions of cells co-expressing two surface antigens of the CD45 antigen family in particular co-expressing CD45/CD45RA or CD45/CD45RO does not exceed 5%, in particular not 2% or 1% or 0.5%.

Evidently, the portions of non-hematopoietic stem and progenitor [stem] cells obtained in the cell product according to the second aspect of the invention as measured by cell cytometry in numbers of cells expressing a particular surface antigen indicative of a non-hematopoietic stem cell type among the total number of cells in the cell product is dependent on the number of cells expressing the particular surface antigen in the original cell population.

A further characteristic parameter for the cell product is the ratio or percentage ratio of the portions of cells expressing the particular surface antigen in the cell product also termed “positive portion in the cell product” to the portion of cells expressing the particular surface antigen in the original cell population prior to the depletion of hematopoietic cells, also termed “positive portion in the original population”. In exemplary embodiments of the cell product of the second aspect of the invention, which are described further below in Example 8, where the depletion of hematopoietic cells is performed according to an exemplary embodiment of the method of first aspect of the invention, this ratio of positive portions in the cell product over positive portions in the original cell population is termed C/A (or % C/A ratio).

FACS cytometry of the cell product demonstrated the effective elimination of B cells precursors and B cells, T cells precursors, NK cells precursors and monocyte cells precursors expressing or coexpressing in particular CD34/CD45, CD45/CD45RA, CD45, CD45RA, CD45RO, CD7³/₄5, CD19, CD14 and other surface markers.

However, as described above the efficiency of removal of the target cells by immuno-depletion depends on the level of expression of the surface antigens which are selected for the immuno-labeling. The level of expression of surface antigens may range from very strong expression, moderate expression, weak expression, very weak expression to no expression depending on multiple parameters such as age, disease, tissue type, differentiation stage etc. Therefore, the composition of the cell product obtained after depletion in general depends on the expression of surface antigens by the target cells in the original population of cells to be removed by depletion.

Accordingly the composition of the cell product e.g. as measured by the fraction of cells expressing any particular surface antigen by FACS analysis also generally exhibits a large variation from e.g. from patient to patient or tissue to tissue etc. In contrast, when applied to the same cell population, the method of the invention is highly reproducible. Similarly, the % C/A ratio for cell surface antigens may vary from patient to patient even if the same tissue such as bone marrow was donated for the original population of cells. The % C/A ratios are influenced by qualities of the original population of cells such as e.g. by the absolute number of particular cell types or cells expressing a particular surface antigen, or e.g. by the relative portions of particular cell types among all cells in the original population. Such qualities of the original population of cells may in particular be influenced by the tissue source or by the stage of health or by the genetic background of the individual from which the original population of cells is derived. It is known that e.g. the bone marrow of patients affected with disease, in particular with certain autoimmune diseases, exhibits differences with respect to the cell numbers and relative portions of cell types expressing particular to surface antigens in the whole population of bone marrow cells.

The wide variations in the cellular composition measured by FACS analysis is reflected also in Tables A and B where ranges of general and preferred percentage ratios of the portions of positive cells expressing a particular surface antigen in the original population of cells versus the cell product (% C/A ratio) are indicated. In particular and as described above, the cell product may exhibit surprisingly large portions of cells expressing a marker of the CD45 family, which inter alia is expressed on granulocytes and granulocyte precursors. CD11B and CD15 are also markers expressed by granluopoietic cells and granulocytes. The portion of granulocytes expressing CD11B and CD15 in the final product vs the original population of cells is preferably decreased. However, granulocytes are cells of the hematopoietic system which do not disturb the therapeutic effect of the cell product according to the described method. They may even enhance the therapeutic effect, because they are known to promote repair of tissue damage (see e.g. Gustafson et al. A Method for Identification and Analysis of Non-Overlapping Myeloid

Immunophenotypes in Humans. PLOS ONE|DOI:10.1371/journal.pone.0121546 Mar. 23, 2015 or Allan, David S. and Strunk, Dirk, 2004, “Regenerative Therapy Using Blood-Derived Stem Cells (Stem Cell Biology and Regenerative Medicine).

CD44 is a further surface marker which is expressed on granulocytes and also on many types of hematopoietic and non-hematopoietic stem cells and progenitor cells. The deliberate object of the method of the invention is to obtain a cell product rich in non-hematopoietic stem cells by depletion of hematopoietic cells and cells of hematopoietic lineage. However, the presence CD44 positive cells including granulocytes such as in particular myelocytes, metamyelocytes and band cells of the hematopoietic system in the cell product is tolerable or even advantageous.”

Indeed, the method of the invention may exhibit percentage ratios of portions of CD44 positive cells in the cell product versus the original population of cells (% C/A ratio) of at least 7% or at least 30% or in particular of at least 7% to at most 30% or more particularly of 7% to 25%.

FACS cytometry analysis of the cell product included single stain analysis with anti-CD45 and anti-CD14 each alone and together in double stain analysis as well as control gate analysis for identification of the types of granulocytes such as myelocytes, metamyelocytes and band cells which are CD14 negative.

It was observed that in the final products obtained from different healthy controls and patients the portions of cells expressing CD11B, CD15 and CD44 highly correlated to the portions of cells expressing CD45.

This is consistent with the known co-expression of these surface markers on myelocytes, metamyelocytes and band cells which are precursors of granulocytes as published e.g. by Attar Attar A., Global Journal of Hematology and Blood Transfusion, 1, 20-28, 2014.

It is further noted that MS patients and generally patients suffering from autoimmune diseases have an elevated level of granulocytes. In case of MS patients it is known that they have elevated IgE level in the blood and also bone marrow. While standard concentrations are smaller than 100 IU/ml in MS patients, it averages around 125 IU/ml in MS patients in clinical experiments of Example 8 and values up to 223 IU/ml were measured. IgE is known to stimulate the bone marrow to produce granulopoietic cells. Furthermore, autoimmune disease patients in general exhibit elevated levels of granulocytes, in particular eosinophils. This additionally explains the presence of granulocytes in the autologous cell product derived from MS patients and prepared according to the method of the invention.

Table B below lists several cell surface antigens correlated to at least some of the relevant cell types on which they are expressed and it lists furthermore generally observed and preferred percentage ratios of portions of positives in the cell product vs the portions of positives in the original cell population (% C/A ratio), in particular relating to an original cell population derived from bone marrow as observed in embodiments of the cell product obtained by depletion of hematopoietic cells, in particular obtained according to embodiments of the method of the invention.

TABLE B

| | Cell Types | | | | Percentage Ratio of Portions of | |
|---|---|---|---|---|---|---|
| | | | | Undesired cell | Positives in Cell Product vs. | |
| | Desired non-hematopoietic | | | types | Original Population | |
| Surface | stem cell types | | | Hematopoietic | General | Preferred |
| Antigen | Pluripotent | Multipotent | Progenitor | cells | %-Ratio | %-Ratio |
| SSEA-4 | ● | | | | ≥25-65 | ≥40, ≥75 |
| 90 | ● | ● | ● | | ≥10-40 | ≥25, ≥50 |
| 133 | ● | ● | ● | ▾ | ≥10-30 | ≥20, ≥40 |
| 34/133 | ● | ● | ● | ▾ | ≥10-50 | ≥30, ≥70 |
| 44 | | ● | | ▾ | ≤30 | 7-25 |
| 11B | | | | ▾ | ≤70 | ≤25-30 ± 5 |
| 15 | | | | ▾ | ≤65-70 ± 5 | ≤40 ± 5 |
| 71 | | ● | | | ≥20-60 | ≥40, ≥75 |
| 73 | | ● | | | ≥3-10 | ≥7, ≥15 |
| 105 | | ● | ● | | ≥17-50 | ≥35, ≥65 |
| 106 | | ● | | | ≥3-9 | ≥6, ≥12 |
| 117 | | ● | ● | ▾ | ≤2-30 | ≤5, ≤15 |
| 146 | | ● | ● | | ≥20-60 | ≥40, ≥75 |
| 166 | | ● | | | ≥12-35 | ≥25, ≥50 |
| 34 | ● | ● | ● | ▾ | 2-30 | 2-20, 5-30 |
| 14 | | | | ▾ | ≤5-25 | ≤10, ≤18 |
| 19 | | | | ▾ | ≤1-20 | ≤5, ≤12 |
| ICAM-1 | | | | ▾ | ≤1-20 | ≤5, ≤12 |
| 34/45 | | | | ▾ | ≤1-25 | ≤5, ≤12 |
| 73/45 | | | | ▾ | ≤1-25 | ≤5, ≤12 |
| 45 Family | | | | ▾ | ≤0-40 | ≤5, ≤10, |
| | | | | | | ≤20, ≤30 |
| 45/45RA | | | | ▾ | ≤0-10 | ≤4, ≤7 |
| 45RA | | | | ▾ | ≤0-15 | ≤5, ≤10 |
| 45RO | | | | ▾ | ≤0-15 | ≤5, ≤10 |

In some embodiments of the cell product obtainable by in vitro depletion of hematopoietic cells from a tissue probe according to the second aspect of the invention, the ratios of portions of positives in the cell product versus the original cell population are within the general or preferred ranges for one, or for more than one, or for particular combinations of the surface antigens listed in Table B.

In some embodiments of the second aspect the cell product is characterized by values for the ratios of portions of positives in cell product vs. original population which differ from the values indicated in table B with respect to particular surface antigens.

In some embodiments one or more of the percentage ratios of portions of positives in the cell product versus in the original cell population of pluripotent stem cells such as cells expressing SSEA-4 or CD90 or CD133 or cells co-expressing CD34/CD133 amount to at least 10% or at least 20% or 30% or 50% or 75%.

In some of these and further embodiments one or more of the percentage ratios of portions of positives in the cell product versus in the original cell population of multipotent stem or progenitor [stem] cells expressing one or a combination of the surface antigens CD90, CD133, CD44, CD71, CD73, CD105, CD106, CD117, CD146 CD166, CD34 or in particular cells co-expressing CD34/CD133 amount to at least 5% or 10% or at least 20% or 30% or 50% or 75%.

Furthermore, in these and further embodiments the percentage ratios of portions of positives of cells in the cell product vs in the original population of cells expressing CD34 or expressing a surface antigen of the CD45 surface antigen family does not exceed 40% or in particular does not exceed 30%, 20% or 10% or 5%.

Furthermore, in these and further embodiments the percentage ratios of portions of positives in the cell product versus in the original cell population expressing one of the surface antigens CD14, CD19, ICAM-1 or co-expressing CD45/CD34 does not exceed 25% or in particular does not exceed 20%, 15%, 10%, 5%, 2% or 1%.

Furthermore, in these and further embodiments the percentage ratios of portions of positives in the cell product versus in the original cell population expressing an antigen of the CD45 surface antigen family such as CD45, CD45RA, CD45RO does not exceed 40% or in particular does not exceed 30%, 20% 15%, 10%, 5%, 2% or 1%.

A third aspect of the invention relates to a cell product comprising non-hematopoietic progenitor [stem] cells, multipotent stem cells and pluripotent stem cells and in particular mesenchymal stem cells according to the second aspect of the invention, in particular obtained from bone marrow, by in vitro depletion of hematopoietic cells for medical therapy, in particular for medical regeneration of lost or damaged tissue and in particular for the treatment of autoimmune diseases and/or neurological diseases. The donated tissue may stem from diverse sources including besides bone marrow e.g. blood, adipose tissue, umbilical cord and other tissues, which may be homologous or heterologous. In particular, the third aspect of the invention relates to the cell product for use in the treatment of degenerative neurological diseases and/or in the treatment of autoimmune diseases. In particular it relates to treatment of diseases like multiple sclerosis, diabetes mellitus type I and type II, rheumatoid arthritis, myocardial infarction and ischemic stroke.

A fourth aspect of the invention relates to a pharmaceutical formulation comprising the cell product according to the second or third aspect of the invention.

### BRIEF DESCRIPTION OF THE FIGURES

The invention will be better understood and objects other than those set forth above will become apparent when consideration is given to the following detailed description thereof. This description makes reference to the annexed drawings, wherein:

FIGS. 1 and 2 regard a mouse model for rheumatoid arthritis, in particular

FIG. 1 shows the results of foot print analysis of three groups of mice treated by administration of with various amounts of a cell product of an exemplary embodiment the present invention as well as an untreated group and a healthy control group;

FIG. 2 shows the changes in clinical symptoms of a female group of mice after administration of an exemplary embodiment of the inventive cell product;

FIG. 3 regards a mouse model for diabetes type I and shows the results of analysis of both treated and untreated groups of mice as well as healthy control groups, in particular

FIG. 3.1 shows glycemia (blood glucose levels);

FIG. 3.2 shows glycated hemoglobin as a marker for average blood glucose levels over the previous three months;

FIG. 4 regards a mouse model for diabetes type II, in particular

FIG. 4 shows the results of analysis of glycemia (blood glucose levels) in both treated and untreated groups of female mice as well as in a healthy control group

FIG. 5 regards a mouse model for ischemic stroke, in particular

FIG. 5.1 and FIG. 5.2 show the results of analysis of neurological deficits in both treated groups IIA and IIC, respectively in comparison with untreated group III.

FIG. 6 regards a mouse model for myocardial infarction, in particular

FIG. 6 shows the size of the surface area of the post infarction heart scar of both treated and untreated groups of mice as well as healthy control groups of male mice as measured by the collagen content in the heart.

FIG. 7 regards a mouse model for multiple sclerosis. The FIGS. 7.1 to 7.5 show the presence or absence of therapeutic activity of different cell populations which were obtained according to Example 1 and tested with and without in vitro amplification by in vitro culture prior to administration to EAE mice, in particular

FIG. 7.1 shows the effect of freshly obtained fraction C, which is an exemplary embodiment of the inventive stem cell comprising cell product derived from bone marrow and depleted of hematopoietic cells;

FIG. 7.2 shows the effect of freshly obtained fraction D, which is the fraction comprising the selected hematopoietic cells retained by the depletion column and subsequently eluted,

FIG. 7.3 shows the effect of freshly obtained fraction A, which is whole bone marrow, i.e. the original population of cells,

FIG. 7.4 shows the effect of in vitro cultured fraction A (whole bone marrow)

FIG. 7.5 shows the effect of in vitro cultured fraction C, (exemplary embodiment of inventive stem cell comprising cell product depleted of hematopoietic cells)

FIGS. 8 to 10 regard clinical data obtained with three exemplary MS patients, to whom an exemplary embodiment of the cell product was transferred. Data are presented for three time points: shortly prior to transfer of the cell product to the patient (Tr) as well as 12 and 24 months thereafter.

FIGS. 8.1.a, 8.2.a and 8.3.a show for each of the three patients the change in the size of the selected characteristic plaques.

FIGS. 8.1.b, 8.2.b and 8.3.b show for each of the three patients the EDSS score at the corresponding time points.

FIG. 9 regards the average effect by the treatment with the exemplary embodiment of the cell product in the three MS patients on the upper extremities in FIG. 9.1 and lower extremities in FIG. 9.2.

FIG. 10 shows the average of the immunoglobulin levels of the three MS patients in the bloodstream compared to the upper and lower levels of the norm.

### MODES FOR CARRYING OUT THE INVENTION

While there are shown and described presently preferred embodiments of the invention, it is to be distinctly understood that the invention is not limited thereto but may be otherwise variously embodied and practiced within the scope of the following claims.

Donated autologous or heterologous tissue constituting the original population of cells is the starting material for the ex vivo method according to the first aspect of the invention. Methods of obtaining tissue from a donor are known and not subject of the current ex vivo method of the invention. The removed tissue forming the original population of cells is usually obtained in a solution comprising commercially available buffer in particular based on PBS (phosphate buffered saline) which may further comprise e.g. an anticoagulant and/or a stabilizer. Such buffer solutions are commonly used in the art and e.g. present in standard sterile bags for recovery of blood, bone marrow or another tissue.

The duration of time between tissue removal followed by the preparation of the cell product and the therapeutic application of the cell product may preferably be kept short. In particular, without significant loss of the therapeutic activity in the cell product the time between tissue removal and therapeutic application of the cell product may last up to 7 or 9 days, but it is preferably kept below 72 hours, 48 hours 36 or 24 hours, temperatures between 4° C. and 8° C., or below 6° C. or below 5° C. and additionally the tissue is preferably kept in the dark. These conditions are preferably applied during the entire ex vivo handling of the cell population. The viability of cells which is observed in the final product is at least 80% and with very rare exceptions it is even above 90% or 95%. Importantly, this viability of the cells in the final cell product is maintained at the same level for at least 24 hours and then only gradually decreases to a level of at least 80% during the following 9 days, when the cell product in particular is stored in the dark at a temperature of 4 to 8° C. Thus, it is advantageously possible to separate from each other both with respect to time and place the tissue removal, the ex vivo handling including the in vitro depletion and the therapeutic application. Some buffers for storage of cells allow storage at room temperature (19° C. to 25° C.), too.

The cell number and composition of the cell population in the cell suspensions generated from the original cell suspension change progressively along the steps of the method from the original population of cells to the final therapeutically active cell product. The total cell numbers as well as the cell numbers of the various cell types of the cell populations in the suspensions generated during progression of the method may be analyzed e.g. by FACS (Fluorescent activated cell sorting) using commercially available equipment, fluorescent antibodies and kits, such as e.g. an MSC phenotyping kit available from Miltenyi Biotec or similar commercially available products. Some or all of the following and also further surface antigens may be chosen for monitoring the distribution of different cell types in the cell suspension during the method: SSEA-4, CD135, CD166, CD146, ICAM-1, CD11B, CD15, CD19, CD14, CD45, CD44, CD45RO, CD45RA, CD71, CD90, CD73, CD106, CD117, CD105, CD34, CD133, CD10. Further markers may be added e.g. for monitoring cell types desired cell types in the cell product or further surface antigens used for the immuno-depletion.

Evidently, besides deliberate removal and immuno-depletion also unspecific loss of cells occurs during the steps of the method resulting in a reduction of the total number of cells. Accordingly, such loss also affects relative degrees of depletion or enrichment of certain cell types. For example hemolysis during a washing and/or singling procedure contributes to the desired depletion of erythrocytes and plastic adherence may contribute to undesired loss of mesenchymal stem cells.

The original population of cells may be singled yielding an original single cell suspension e.g.

by passing the cell population through a 50 μm to 300 μm filter or mesh cell strainer, in particular through a 70 μm or 80 μm or 90 μm or 100 μm to 150 μm filter or mesh cell strainer or through a 200 μm filter or mesh cell strainer and/or the original population of cells may be washed for removal of dead cells, cell debris and other material present in the obtained tissue sample. The original population of cells may after optional filtering be transformed into a washed suspension by gentle centrifugation e.g. for 10 to 20 min at 300 g to 600 g, in particular at 300 g to 400 g and resuspension in a suitable buffer. Such washing and filtering may involve a substantial loss of cells such as e.g. by removal of cells which are agglomerated into lumps or e.g. removal of red blood cells and/or platelets. Depending in particular on the washing conditions and the tissue source e.g. 20% to 60% of the cells contained in the tissue donation may be lost.

In some embodiments washed and/or filtered single cell suspensions may be directly subjected to the immuno-labeling procedures. In some embodiments the obtained tissue may also be fractionated e.g. by density fractionation, e.g. by layering on Ficoll or on a Ficoll gradient prior to immuno-depletion, although preferably such additional steps are avoided.

The in vitro immuno-depletion of the original or washed and/or singled cell suspension may be performed using several different antibodies where in particular each antibody is specific for one of the surface antigens of the group of selected surface antigens. The term antibody as used in this text includes various types of immunoglobulins such as e.g. IgA, IgG, IgG1, IgG2 or IgM as well as antigen binding fragments of antibodies and antibody derivatives such as antibodies conjugated to a detectable tag e.g. conjugated to a tag via biotin/streptavidin or via a secondary antibody. Commonly used tags include fluorophores, gold and magnetic particles. A large variety of specific antibodies conjugated to a detectable tag are commercially available and suitable for a variety of immuno-depletion procedures.

The immuno-depletion comprises the immuno-labeling procedure labeling cells expressing one or more of the selected surface antigens with specific antibodies and the separation procedure for removal of the immuno-labeled cells from the original cell population in separate steps and/or several specific antibodies may be combined into in one or more combined steps.

In some embodiments the immuno-labeling comprises an immuno-magnetic labeling procedure wherein antibodies are conjugated to a magnetic bead and wherein in the separation procedures for depleting the immuno-magnetically labeled cells a magnetic separation device is used. Such methods are described in the art and corresponding reagents and equipment are commercially available (e.g. CliniMACS® reagents from Miltenyi Biotec). The indirect method for most monoclonal antibodies is more efficient in removing the corresponding selected cells which express a particular selected surface antigen, because the antibodies without magnetic particles find their antigenic target on cell surfaces more efficiently, than antibodies conjugated to magnetic particles. The direct method is generally faster than the indirect method. Both indirect and direct labeling procedures may be performed within one immuno-depletion procedure either in subsequent or in combined steps.

In some of these and other embodiments of immuno-labeling the cell population may be incubated with conjugated antibodies in a direct immuno-labeling procedure, wherein the surface antigen specific antibodies used in the labeling procedure are conjugated to a tag prior to the labeling procedure or are commercially available as such. The resulting selected cells labeled with tag-conjugated antibodies are directly ready for the separation procedure.

Alternatively or in combination with embodiments comprising one or more direct labeling procedures, one or more indirect immuno-labeling procedures may be performed. In the indirect labeling procedure the cell population is first incubated with primary antibodies and subsequently incubated with secondary antibodies or with another conjugation reagent comprising a tag and binding to the primary antibodies according to procedures known in the art. The primary antibodies in particular may be biotinylated antibodies, which are conjugated by streptavidin or by secondary anti-biotin antibodies coupled to a tag, such as a fluorophore or a magnetic bead.

Preferably, after incubation of the cell suspension for binding the primary specific antibody or antibodies to the selected surface antigen(s), the cell suspension is washed for removal of excess unbound primary antibodies, e.g. by centrifugation and re-suspension. The re-suspended cell population is then incubated with reagents for attaching a tag to the primary antibodies.

In some of these and other embodiments the antibodies specific for the selected surface antigens may be incubated with the cell suspension in individual steps each or several surface antigen specific antibodies, in particular less than 10 or less than 6 different antibodies or more particular up to 2 or up to 3 or up to or up to 5 different antibodies, may be combined as an antibody cocktail for simultaneous incubation with the cell suspension. Tag-conjugated antibodies and non-conjugated antibodies and/or tags may be incubated individually or in combined steps. Some embodiments of the immuno-labeling procedure comprise up to 6, in particular up to 3 or up to 2 incubation steps.

In some embodiments ratios of antibodies per number of cells in the cell population, in particular for commercially available reagents for direct and/or indirect labeling procedures may be used with standard incubation conditions e.g. as specified by the manufacturer such as Miltenyi Biotec, BD Biosciences and other suppliers, for individual antibodies and also for individual antibodies pooled into an antibody cocktail.

If non-commercial antibodies are used for the immuno-depletion, an optimally suited concentration of antibodies for incubation with the cell suspension may be titrated according to techniques known in the art. Briefly, a dilution series of a varied number of tag-conjugated or unconjugated primary surface antigen specific antibodies is incubated with a fixed number of cells. Using a suitable detection system such as FACS (Fluorescent activated cell sorting) an optimal ratio of amount of antibodies per number of cells is determined, wherein as many as possible of the cells expressing the specific surface antigen are labeled with the tag and at the same time as few of the cells as possible without the specific surface antigen are labeled by unspecific association of the tagged antibodies to the cell surface.

Similarly, when using indirect labeling after binding of unconjugated primary antibodies to cell surface antigens, the amount of tag such as fluorophore or magnetic beads used for incubation with the cell suspension may be titrated to optimize between maximal amount of tags bound per available primary antibodies and minimal unspecific association of tags with the cells.

The term standard incubation conditions is used in this text for incubation conditions during the immuno-labeling of the method, which e.g. correspond to the manufacturers specifications for the use of immuno-depletion reagents. Such reagents are e.g. monoclonal antibodies, including antibody derivatives, in particular biotinylated derivatives and tags such as flurophores or magnetic beads conjugated derivatives. The term standard incubation conditions is also used in this text for conditions which are optimized for maximal saturation with specifically bound antibodies to corresponding antigenic binding sites of selected surface antigens while keeping non-specific binding of antibodies to cells lacking the specific selected surface antigen at a reasonably low level. In particular, a reasonable level of non-specific association of antibodies or tags may amount to less than 30%, in particular less than 20% or less than 10% or less than 5% or less than 2% of the level of specific binding of antibodies to cells expressing the corresponding surface antigen. Standard conditions as specified by the manufacturer for some embodiments may be expected to be optimized for maximal saturation with specifically bound antibodies to corresponding antigenic binding sites of selected surface antigens while keeping non-specific binding of antibodies to cells lacking the specific selected surface antigen at low level such as a level below 30% as specified above.

In some embodiments standard incubation conditions comprise an antibody concentration for each antibody present in an immuno-labeling step of 0.1 to 2.5 mg antibody per 100 ml +/−10 ml incubation volume, in particular of 0.25 to 0.75 mg, more particularly 0.5 mg antibody per 100 ml +/−10 ml incubation volume. In some of these and other embodiments the number of cells subjected to the immuno-depletion and in particular the number of cells present during the incubations with antibodies against selected antigens does not exceed 10¹⁰ cells or does not exceed 5×10⁹ or 3×10⁹ or 2×10⁹ or 1.5×10⁹ or 1.2×10⁹ or 1.0×10⁹cells. In particular the cells present during the incubations with antibodies against selected antigens ranges from at 10⁵ to 10¹⁰ cells in 100 ml +/−10 ml incubation volume or in particular it ranges from 10⁷ to 5 ×10⁹ cells or from 3×10⁷ to 2×10⁹ cells 100 ml +/−10 ml incubation volume.

In some embodiments of the method of providing the cell product at least one step of the immuno-depletion is performed wherein the degree of depletion is limited, i.e. under limiting conditions, in particular comprising limiting incubation conditions for immuno-labeling and/or limiting separation conditions. Limiting incubation conditions achieve that cells of the original cell population which express a comparatively large number of selected antigenic surface markers per cell are depleted with greater efficiency from the original cell population than cells expressing a comparatively small number of selected surface antigens per cell. This may be achieved e.g. by a reduction of the number of bonds formed between antibodies and surface antigen or between antibodies and tags, e.g. by decreasing the efficiency of the labeling of the selected surface antigens. The efficiency of labeling may be decreased in particular by performing the incubation with the antibodies or the tags under conditions which only allow for a lower number of binding pairs formed between the tag-conjugated antibodies or the primary antibodies and surface antigens or between the tag conjugated reagents (such as streptavidin coated magnetic beads) or tag conjugated secondary antibodies and primary antibodies compared to the number of binding pairs that would form under standard incubation conditions.

In exemplary embodiments with magnetic beads used as tags, the efficiency of labeling may be decreased e.g. by decreasing the incubation temperature, the incubation time or the concentration of the magnetic beads with respect to the manufactures specification when using commercially available reagents or with respect to the optimal conditions obtained from a titration curve determining conditions for maximal binding of magnetic beads to primary antibodies at an acceptable level of non-specific binding of magnetic beads to cells. In some embodiments more than one of these measures can be applied simultaneously.

In some of these and other embodiments of the method the limiting incubation conditions are adjusted to allow for only a partial saturation of the CD34 or of the CD133 or of the CD117 antigenic binding sites, in particular of the CD34 antigenic binding sites, on the cells of the original cell population by corresponding antibodies.

In some of these and other embodiments of the method of the invention, the in vitro immuno-depletion comprises an immuno-labeling procedure which is performed in at least two stages: In a first stage, the cells are labeled with antibodies against selected surface antigens except for antibodies against one or more of the CD34, CD133 or CD117 surface antigens, in particular against the CD34 surface antigen, and in a second stage which is performed after the first stage cells are labeled with antibodies against the surface antigens deliberately excluded in the first stage, i.e. against one or more of the CD34, CD133 or CD117 surface antigens, in particular against the CD34 surface antigen and optionally with antibodies against further selected antigens. Both the first and the second stage may comprise one or more incubation step for immuno-labeling of the cells with individual antibodies separately in separate incubation steps or for immuno-labeling of the cells with an antibody cocktail comprising several antibodies for labeling several selected surface antigens in the same incubation step. Furthermore, both the first and the second stage may comprise direct and/or indirect immuno-labeling. In some of these and other embodiments the first stage comprises or consists of the first step of an indirect immuno-labeling procedure and/or the second stage comprises or consists of a combination of the second step of an indirect immuno-labeling with a direct immuno-labeling with tag-conjugated antibodies against one or more of the CD34, CD133 or CD117 surface antigens in the same incubation step.

Incubation with cocktails comprising several antibodies for labelling two or several, in particular three or four selected surface antigens at the same time are particularly preferred because this accelerates the in vitro depletion procedure. This in turn enhances the physiological and the therapeutic properties of the cell product after the immuno-depletion in particular with respect to the composition of the cell population and the cell viability.

In some of these and other embodiments, in the first stage the selected antigens are comprising CD14, CD45 and at least one further CD45 family member, in particular CD45RA and/or CD45RO, and wherein in the second stage the selected antigen is CD34. In some of these and other embodiments the conditions are adjusted to limit the degree of labeling and therefore also the degree of depletion in particular by increasing the incubation volume in both stages or particularly in the second stage, by a factor of 1.5 to 4, in particular by a factor of 2 to 3.

The lack of antibodies against CD34, CD133 or CD117 in the first stage of the immuno-labeling and in particular by incubating the cells in the second stage with anti CD34, CD133 or CD117 antibodies in a volume of buffer, which is larger than the standard volume specified by the manufacturer results in partial depletion of in particular of CD34 positive cells. This results in an increased portion of cells expressing both CD34 and CD133 which are known to be expressed by pluripotent stem cells and therefore desired cells in the cell product.

In particular it has been observed that the percentage ratios of portions of CD34 positive cells in the cell product versus the original population of cells (% C/A ratio) increases by 5-15%, in particular by 8-10%, when the volume in the second labelling step of the cells with anti-CD34 antibodies coupled to a magnetic tag is increased by a factor of 2 and it increases by 25-40% in particular by 30-35%, if the volume is increased by a factor of 4.0

In some of these and other embodiments with indirect immuno-labeling biotinylated primary antibodies that are conjugated with a tag by means of e.g. tag-conjugated secondary antibodies or e.g. streptavidin-connected tags, like streptavidin coated magnetic particles, are used.

In some of these and other embodiments the immuno-magnetic labeling may comprise the following steps A, B and C, which are not necessarily performed immediately after one another;

in step A of the immuno[-magnetic] labeling, cells are incubated with a cocktail comprising biotinylated antibodies against more than one surface antigens,

in step A incubation conditions are applied to allow for a maximized saturation of at least part of the selected surface antigens while minimizing unspecific binding of antibodies to cells; excess unbound antibodies are removed after step A by centrifugation followed by re-suspension of the cells;

in step B of the immuno-magnetic labeling, cells are incubated with a cocktail comprising biotinylated antibodies against surface antigen,

in step B limiting incubation conditions are allowing for only a partial saturation of at least one surface antigen; excess unbound antibodies are removed by centrifugation followed by re-suspension of the cells after step B;

in step C of the immune-magnetic labeling the cell population is labeled with anti-biotin antibodies conjugated to magnetic particles,

in step C limiting incubation conditions are allowing for only a partial saturation of antigenic biotin binding sites by the secondary anti-biotin antibodies; excess unbound antibodies are removed by centrifugation followed by resuspension of the cells after step C.

In some embodiments steps B and C of the immune-magnetic labeling may be combined. In such combined step B/C (which may also just be named step B), the cell population is incubated with anti-biotin antibodies conjugated to magnetic particles and with antibodies conjugated to magnetic particles and directed against at least one selected surface antigen during the same incubation step followed by one step of removal of excess unbound antibodies by centrifugation and re-suspension of the cells after the combined step B/C.

In the separation procedure applied after steps A to C or A and B, respectively, cells labeled with at least two or at least three or at least four bound magnetic particles are removed.

Exemplary combinations of antibodies have been described in the Experiments. They comprise e.g. anti-CD14, and anti-CD45 family antibodies used as primary antibodies, e.g. in step A above, and anti-CD34 antibodies conjugated to a magnetic bead used e.g. in step B and anti-biotin antibodies conjugated to a magnetic bead used e.g. in step C, wherein step B and step C may optionally be combined into one step.

In some of these and other embodiments comprising immuno-magnetic depletion the separation conditions may be limited such that labeled cells which have less than two or three or four magnetic particles bound are not removed by the magnetic separation device. The efficiency of removal of magnetically labeled cells may be gauged e.g. by using electromagnetic separation devices with an adjustable magnetic field strength or by increasing a distance of the cell suspension to the magnetic device resulting in a lower magnetic field exerted on the magnetically labeled cells to a desired level.

In some of these and other embodiments comprising a step of immuno-depletion with limiting conditions, the partial saturation of the antigenic binding sites is achieved by reducing the contact efficiency between antibody and antigen or by reducing the binding probability between the antibody and antigen. This may be performed e.g. by choosing one or a combination of the following conditions:
by increasing the incubation volume by a factor of 1.5 to 4 in particular by a factor of 2;
by reducing the incubation time;
by adapting the incubation temperature;
by adapting the moving conditions of a rotator or shaker used during the incubation, e.g. the moving speed;
by lowering the ratio of amount of antibodies to number of cells.

In some of these and other embodiments of the method according to the invention, the portion of selected cells in the cell product expressing at least one surface antigen of the group of selected surface antigens, in particular of the selected surface antigens which are characteristic of hematopoietic cells, is reduced by a factor of at least 2, 3, 5, 10, 50 or 100 compared to the original population of cells, and wherein the group of selected hematopoietic surface antigens includes CD14, CD19, CD34, CD45 family, and ICAM-1.

In some embodiments in particular a portion of cells which are “double-positive”, i.e. expressing two of the selected surface antigens such as CD45+/CD34+, CD45+/CD45RA+, CD45+/CD45RO+ or CD45+/CD14+ are depleted with an absolute degree of depletion of below 0.2, in particular to below 0.1 or 0.05 or 0.02 or 0.01 corresponding to a factor of absolute depletion over 5, 10, 20, 50 or 100 fold.

In some of these and other embodiments the portion of cells in the cell product expressing at least one of a group of surface antigens characteristic of non-hematopoietic stem and progenitor cells, in particular characteristic of multipotent stem cells, pluripotent stem cells or in particular mesenchymal stem cells, is increased by a factor of at least 2, or at least 3, 5, 10, or 100, compared to the original population of cells. The group of non-hematopoietic surface antigens and in particular of mesenchymal stem cell antigens includes e.g. SSEA-4, CD90, CD133, CD71, CD73, CD105 and CD106.

Surface antigens which are characteristic for particular cell types are known in the art. Some information in this respect is provided in this text and also in some of the included references. Further corresponding information may also be retrieved from the internet, scientific literature and commercial institutions as applicable for updating to current knowledge or adjusting to particular applications regarding e.g. the donor tissue or the therapeutic application.

The second aspect of the invention relates to the cell product obtainable by the method according to the first aspect of the invention. The advantageous properties, in particular the surprising therapeutic activity of the cell product are described above.

The cell population obtained as therapeutically active cell product may be further washed, purified and prepared for use as a pharmaceutical composition according to the third aspect of the invention. In some embodiments, prior to administration, the cell product, preferably as obtained after depletion without in vitro cultivation (see below), may be suspended in a physiologic isotonic solution, which may be chosen to be particularly suitable for the intended therapeutic administration such as systemic intravenous administration, lumbar puncture, direct injection into a particular organ or administration during a surgical procedure.

The cell product may e.g. be suspended in a PBS/EDTA buffer comprising 0.5% human serum albumin. For a cell product obtained from about 50ml of bone marrow, buffer to e.g. a final volume of about 150 ml proved suitable. This concentration, however, can be adjusted dependent on the actual cell number in the cell product. The concentration of cells may be diluted with 0.9% saline solution to a concentration not much greater than approximately 10⁶ cells per ml to e.g. 0.1 to 5×10⁶ cells/ml or in particular 0.5 to 1.5×10⁶ cells/ml shortly before transfer of the cell product as pharmaceutical composition to the patient, e.g. by systemic intravenous administration. In some embodiments between 1 and 10×10⁶ cells per kg body weight of the patient or in particular between 2 and 6×10⁶ or between 2 and 8×10⁶ are administered. In particular embodiments up to 2×10⁶ cells or 2 to 4×10⁶ cells or 4 to 6×10⁶ cells per kg body weight are administered e.g. by intravenous infusion.

In some embodiments the therapeutically active cells of the cell product are bone marrow derived, in particular ilium derived, autologous non-hematopoietic stem cells. During the process of preparation of the cell product, the extracted bone marrow undergoes non-substantial in vitro manipulations only, such as filtration, washing/centrifugation and cell separation by depletion of hematopoietic cells and optionally further cells. Furthermore, after the depletion of hematopoietic cells, the cell product is transferred to the patient preferably without prior amplification in vitro. The number of cells obtained in the cell product is usually sufficient, although it may vary depending in particular on the tissue source and the donor.

The cell product for medical use according to the third aspect of the invention as described may be used by different ways of administration and for a number of different medical indications, in particular to regenerate lost or damaged tissue, and in particular for the treatment of degenerative neurological disease and/or the treatment of autoimmune disease and in particular for the treatment of multiple sclerosis, diabetes mellitus type I and type II, rheumatoid arthritis, myocardial infarction and ischemic stroke.

### EXAMPLES

### SECTION A: Experiments in Mice

A preliminary set of experiments was performed with mice. With this preliminary set of experiments the concept of the method for providing a therapeutically active cell product which comprises non-hematopoietic stem and progenitor cells in a state and cellular environment which is as physiological as possible was originally developed and tested. Care was taken to perform all of the ex vivo steps including the in vitro depletion of hematopoietic cells as gently as possible for minimizing damage to or loss of the small number of the fragile non-hematopoietic stem and progenitor cells present in the original cell population and further for minimizing non-physiological adaptations to the ex vivo environment by those cells. It was aspired to obtain physiologically healthy non-hematopoietic stem and progenitor cells in a large enough number to avoid in vitro amplification prior to therapeutic administration in the cell product to provide for therapeutic activity.

Example 1 is an exemplary embodiment of the method for providing the cell product with tissue probes of murine bone marrow. In Examples 2 to 7, the therapeutic activity of the obtained cell product was tested in several murine disease models. For each disease, a large number of mice (approx. 100 to 250) had to be sacrificed, for obtaining enough pooled bone marrow which was subjected to an exemplary embodiment of the ex vivo method. The thereby obtained cell product was subsequently intravenously administered to groups of mice affected by the same disease with variable amounts of the cell product. The therapeutic activity of the cell product was analysed by established tests for the model diseases below. All animal experiments had the permission of the local bioethics committee. The results of these experiments in mice were submitted to the European Medicines Agency on the basis of which it granted the permission for the clinical studies described in SECTION B.

### Example 1: Providing Therapeutically Active Cell Product from Murine Tissue

It is a well-known fact in the art, that the cell surface antigen profiles of human and murine cells do not fully correspond (e.g. Phinney and Sensebé, 2013). Accordingly, for murine tissue a different group of selected surface antigens is suitable for removal of hematopoietic cells by in vitro depletion and recovery of non-hematopoietic stem and progenitor cells in the cell product compared to human tissue which sets limits for the applicability of experiments in mice to humans.

Yet, the basic steps of the in vitro treatment of the isolated tissue in the method for the provision of the cell product with murine and human tissue are the same. Exemplary embodiments of the method performed with murine tissue may comprise the following steps, as used here in Example 1:
filtration of the isolated bone marrow obtained from mice;
purification of isolated marrow (washing/centrifugation);
first labelling step with biotinylated monoclonal antibodies (against selected cell surface antigens);
removal of unbound antibodies by centrifugation and resuspension;
second labelling step with anti-biotin antibodies conjugated to superparamagnetic iron dextran particles and optionally further antibodies conjugated to superparamagnetic iron dextran particles which specifically bind to one or more further cell-surface antigen;
removal of unbound antibodies by centrifugation and resuspension of the cells;
depletion of labelled cells using e.g. a CliniMACS magnetic separation device of Miltenyi Biotec (the negative fraction collected as the final product).

In further exemplary embodiments, the number of immuno-labelling steps may be different and in n particular it may comprise e.g. 1 or 2 or 3 or 4 immuno-labelling steps and the method may comprise direct or indirect immuno-magnetic labelling or both.

In Example 1 the cell surface antigens chosen by the inventors for immuno-depletion of hematopoietic cells from murine bone marrow were selected in particular because of their characteristic expression on the cell types listed behind the antigen:
CD5: T lymphocytes, B lymphocyte sub-populations;
CD45R (B220): precursors of and mature B lymphocytes;
CD11b: granulocytes, monocytes, macrophages, dendritic cells, NK cells, B-1 lymphocytes;
Ly-6G (Gr-1): precursors of and mature granulocytes, monocytes, neutrophils;
7-4, Ter-119: precursors of and mature erythrocytes;
Sca-1—(Ly6A/E or Ly6D): immature hematopoietic progenitor cells and hematopoietic stem cells.
CD14—macrophages, dendritic cells, Kupffer cells, hepatocytes, and granulocytes

Antibodies used in this exemplary embodiment and many more are available commercially from a large number of commercial suppliers (see e.g. http://www.antibodyresource.com/onlinecomp.html). Also, buffers, reagents and equipment for immune-magnetic depletion are commercially available e.g. from Miltenyi Biotec and other suppliers. In further exemplary embodiments antibodies against additional or alternate surface antigens may be used for depletion or partial depletion of hematopoietic stem cells and/or further antigens depending on the particular disease model studied.

In these and other embodiments monoclonal antibodies of the IgG2 subfamily may be chosen, in particular for antibodies to antigens of the CD45 family, such as for highly glycosylated CD45RA since antibodies of the subclass IgG2 exhibit enhanced binding to polysaccharides compared to antibodies of the IgG1 subclass.

In these and other exemplary embodiments, the choice of antigens in the group of selected antigens may be adapted to the cellular composition of the original cell population and according to known correlations between the expression of cell surface antigens and cell types to achieve the selective removal of hematopoietic cells and optionally further cell types from the original cell population.

Detailed description of an exemplary embodiment of the method for providing a therapeutically active cell product from bone marrow of mice.

1. Isolation of bone marrow from mice as starting material for an exemplary embodiment of the method of providing a therapeutically active cell product:

Bone marrow was obtained from femur and tibia of mice of an appropriate strain, which have been treated to induce a particular model disease and of healthy control mice of the same strain under sterile conditions at 4° to 8° C. comprising the steps of:
Cutting the base of femur and tibia off and washing out the marrow cavity with PBS (without Ca²⁺, Mg²⁺)
The isolated and pooled bone marrow was suspended in PBS (without Ca²⁺, Mg²⁺) and passed through a nylon filter with a mesh size of 40 to 70 μm and labelled as Fraction A.
The cell suspension was centrifuged (400g, 10 min.; Room Temp) and the supernatant discarded.
The cell pellet was suspended and incubated in erythrocyte lysis buffer (5 ml erythrocyte lysis buffer: 150 mM NH₄Cl, 10 mM KHCO₃, 0.1 mM Na₂ EDTA, pH 7.2)for 5 min. Lysis was stopped by adding PBS (without Ca²⁺, Mg²⁺).
The cell suspension was centrifuged (400×g, 10 min.; Room Temp).
The cell pellet was re-suspended in PBS (without Ca²⁺, Mg²⁺), then passed through a nylon filter with a mesh size of 40 to 70 μm.
The cell suspension was centrifuged (400g, 10 min.; Room Temp.), re-suspended in PBS without Ca²⁺, Mg²⁺) and the total number of cells was determined.

2. In vitro immuno-magnetic depletion of hematopoietic cells from the original cell population:

The whole process was performed in a sterile laminar flow hood chamber and all steps related to the treatment of bone marrow cells were performed under sterile conditions on ice at 4°-8° C., except for the centrifugation steps that were optionally performed at temperatures between 4° C. and room temperature.
The total number of bone marrow cells subjected to the following steps was determined.
The cell suspension was centrifuged (400×g, 10 min.; Room Temp.) and the supernatant discarded.
The cells were suspended in a volume A of buffer B,
   where the volume A is adjusted such that a total incubation volume of 40 μl per 1×10e7 cells was obtained after the addition of antibody cocktails;
   and where the buffer B was PBS without Ca²⁺ and Mg²⁺, 2 mM EDTA, 0.5% BSA.

2.1 A first step of immuno-[magnetic-]-labelling was performed with biotinylated monoclonal antibodies:
Add 10 μl of antibody cocktail No. 1 per 1×10e7 total cells
Where antibody cocktail No. 1 contains CD5; CD45R (B220); CD11b, Anti-Gr-1 (Ly-6G/c); 7-4; Ter-119 each at a concentration for providing excess antibodies at a concentration recommended for immuno-depletion by the manufacturer which is designed for providing excess antibodies. Add 10 μl (=the same volume as cocktail 1) of antibody cocktail No. 2 containing monoclonal antibodies against CD14 (IgG1); CD45 (IgG1); CD45RA (IgG2) each at a concentration of 1 mg/ml.
The suspension was mixed well and incubated for 10-15 min. at 4-8° C. in the dark.
The incubation was stopped by addition of 1 to 2 ml of buffer B per 1×10e7 cells. If the cell number was low (1×10e7 or less) just 2 ml of buffer B was added. Centrifugation of the cell suspension (400 g, 10 min.; Room Temp.).
Cells were suspended in 30 μl of buffer B per 1×10e7 total cells. If the cell number was low (i.e. 1×10e7 or less) 30 μl to 60 μl of buffer B was added.

2.2 A second step of immuno-magnetic-labelling was performed with anti-biotin antibodies conjugated to iron dextran micro beads:
Add 20 μl antibody cocktail No. 3 containing anti-biotin antibodies at a concentration recommended for immuno-depletion by the manufacturer per 1×10e7 cells. If the cell number is low (1×10e7 or less) just 20 μl of antibody cocktail No. 3 were added.
The suspension was mixed well and incubated for 10-15 min. at 4-8° C. in the dark.
1-2 ml of buffer B was added per 1×10e7 total cells to stop the incubation process with antibodies. If the cell number was low (1×10e7 or less) just 2 ml of buffer B was added and labelled as fraction B.

2.3 Magnetic separation of the immuno-magnetically labelled cells
Centrifugation (400×g, 10 min.; Room Temp.) and the supernatant was discarded.
Addition of 50 μl of buffer B per 1×10e7 total cells to the pellet rendered the samples ready for magnetic separation. If the number of cells was low (1×10e7 or less) 50 μl to 100 μl of buffer B were added.
A column LS (Miltenyi Biotec Cat. No 130-042-401) was placed in a magnetic separator and rinsed with 3 ml of buffer B. As magnetic separator e.g. MidiMACS™ Separator Cat. No 130-042-301 or QuadroMACS™ Separator Cat. No 130-091-051 or VarioMACS™ Separator, in combination with an LS Column Adapter Cat. No 130-090-282 was used.
After the 3 ml buffer B had run through the column the cell suspension was applied to the column.
Then the column was washed three times with 3 ml buffer each time.
The cells which passed through the column were collected in a new tube and named fraction C, i.e. the negative fraction which was used for intravenous transfer in the subsequent therapeutic experiments of Examples 2 to 7.
The cells retained in the column were eluted from the column outside of the magnetic field and collected in a separate tube. This fraction was named D and represents the magnetically labeled hematopoietic lineage positive cells.

The cell population in the cell product was tested for removal of hematopoietic cells, presence of non-hematopoietic stem cells and cell viability (data not shown).

3. Overview over the mice used for ex vivo provision and in vivo tests for therapeutic activity of the cell product

In Examples 2 to 7, the cell product obtained according to Example 1 was tested for its therapeutic activity in several murine disease models. The selected model diseases rheumatoid arthritis (RA), diabetes mellitus Type 1 (DB1), diabetes mellitus Type 2 (DB2), ischemic stroke (IS) and myocardial infarction (MI) and experimental auto-immune encephalitis (EAE) as model disease for multiple sclerosis (MS) were induced by established treatments in suitable mouse strains. The cell product which was obtained in the ex vivo method from pooled bone marrow of diseased mice was subsequently intravenously administered to groups of mice affected by the same disease with variable amounts of the cell product (1 to 5×10⁶ cells per mouse) according to the Table 1 below. The therapeutic activity of the cell product was analysed by established tests for each of the model disease.

TABLE 1

| | Model Disease | | | | | |
|---|---|---|---|---|---|---|
| | Mice | | | | | |
| | | | | | | Experimental |
| | Rheuma- | Diabetes | Diabetes | | | Auto- |
| | toid ar- | mellitus | mellitus | Ischemic | Myocardial | immune |
| | thritis | Typ 2 | Typ 1 | stroke | in-farction | Encephalitis |
| | Example 2 | Example 3 | Example 4 | Example 5 | Example 6 | Example 7 |
| | strain | | | | | |
| | | | | | | SJL/J |
| number | DBA/1 | C57BL/6 | C57BL/6 | C57BL/6 | BALB/c | Female only |
| total No. | 362 | 362 | 362 | 210 | 382 | 72/302 |
| diseased and | | | | | | |
| control mice | | | | | | |
| used for | 230 | 230 | 230 | 100 | 228 | 36/156 |
| preparation of | | | | | | |
| cell product | | | | | | |
| treated with | 26 | 26 | 26 | 20 | 26 | 18/78 |
| 1 × 10⁶ cells/ | | | | | | |
| mouse (IIA)*** | | | | | | |
| treated with | 26 | 26 | 26 | 20 | 26 | — |
| 3 × 10⁶ cells/ | | | | | | |
| mouse (IIB)* | | | | | | |
| treated with | 26 | 26 | 26 | 20 | 26 | — |
| 5 × 10⁶ cells/ | | | | | | |
| mouse (IIC)** | | | | | | |
| untreated mice | 26 | 26 | 26 | 20 | 28 | 18/68 |
| (=III) | | | | | | |
| healthy control | 28 | 28 | 28 | 10 | 28 | — |
| mice (=IV) | | | | | | |
| sham | — | — | — | 20 | | — |
| induction of | | | | | | |
| stroke | | | | | | |
| confirmation | — | — | — | — | 20 | — |
| of infarction | | | | | | |
| induction | | | | | | |

*except in Rheumatoid arthritis 2.5 × 10⁶ cells/mouse
**except in Rheumatoid arthritis 3.5 × 10⁶ cells/mouse
***except in EAE 2.0 × 10⁶ cells/mouse

### Example 2: Rheumatoid Arthritis

In Example 2, the therapeutic activity of the cell product prepared according to Example 1 was tested in vivo in the experimental model of rheumatoid arthritis in mice.

The experimental model of rheumatoid arthritis (RA) as described in Simon P. Brooks & Stephen B. Dunnett, Nature Reviews Neuroscience 10, 519-529 (July 2009) was induced in mice of both sexes of the strain DBA/1 at the age of 10-11 weeks by subcutaneous administration of an emulsion of chick type II collagen (2-4 mg/ml) and complete Freund's adjuvant (1 mg/ml Mycobacterium tuberculosis), in a dose of a volume of 50 μl at the tail at a distance of 1.5-2 cm from the base. On day 21 after the first immunization a booster dose was given comprising chicken type II collagen (2-4 mg/ml) with Freund's incomplete adjuvant, and in a dose volume of 50 μl.

Three groups (Group IIA, IIB, IIC) of 26 diseased mice each were treated at 30-35 days after the first collagen type II administration (thus aged 15-16 weeks) with the cell product obtained in Example 1:
Group IIA—(1×10⁶ cells/mouse)
Group IIB—(2.5×10⁶ cells/mouse)
Group IIC—(3.5×10⁶ cells/mouse).
Also a control Group III of 26 mice, in which RA was induced, was left untreated and a further control Group IV of 28 healthy mice of the same strain and age in which the Rheumatoid arthritis disease was not induced were subsequently evaluated together with the treated mice of Groups II according to the same diagnostic parameters.

The following diagnostic parameters were evaluated on the in vivo mice model of rheumatoid arthritis: C-terminal telopeptide of type II collagen (CTx II), matrix metalloproteinase type 3 (MMP-3), cartilage oligomeric matrix protein (COMP) and IgG1 and IgG2a immunoglobulins (data not shown).

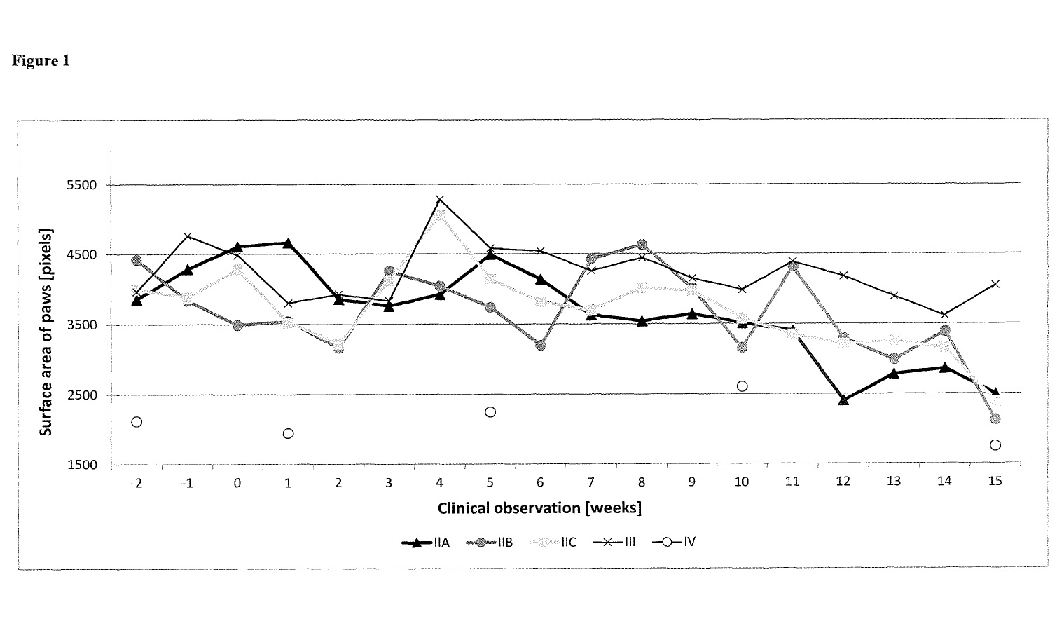

Furthermore, as shown in FIG. 1 footprint analysis was performed as described in Simon P. Brooks & Stephen B. Dunnett (Nature Reviews Neuroscience 10, 519-529,July 2009) to assess the presence and course of the RA disease in both treated and untreated mice and compared to healthy controls of the above mentioned Groups II, III and IV. The y-axis shows the automatically measured area in pixels of the footprints generated by the mice whose front sole of the hind paws was immersed into ink when they walked through a narrow experimental corridor. The x-axis shows the time of observation in weeks. The 0-time point marks the administration of the cell product to mice of Groups IIA, IIB and IIC in amounts (cell number per mouse) according to Table 1. Data were collected beginning two weeks before beginning the treatment by administration of the cell product which is corresponding to the time point of administration of the booster shot with type II collagen.

As FIG. 1 shows, healthy control mice (Group IV) generate a footprint area in a range of approx. 1800 to 2600 pixels whereas mice in which the disease was induced generate approx. 3200 to 5300 pixels both without treatment and with treatment during the first approx. 11 weeks after administration of the cell product.

Around week 12 the effect of the treatment can clearly be seen in the treated Groups IIA, IIB, IIC with the footprint area dropping to values below 3000 pixels and even below 2500 pixels, thus approaching the range scored by healthy control mice, whereas the untreated mice continue to score values of approx. 3600 to 4100 pixels.

Additionally, the severity of the clinical symptoms of RA was assessed using a five-point scale as described by Brand DD et al., Collagen-induced arthritis. Nat Protoc. 2007;2(5):1269-75 and by Seeuws S et al., A multiparameter approach to monitor disease activity in collagen-induced arthritis Arthritis Res Ther. 2010;12(4):R160 with the scale:

0—no symptoms of the disease;

1—one toe inflamed and swollen;

2—more than one toe inflamed and swollen (but not the whole paw) or mild swelling of the entire paw;

3—the whole paw inflamed and swollen;

4—severe inflammation and stiffness including toes, foot and ankle, which symptoms prevent the mouse to grip the wire cage cover.

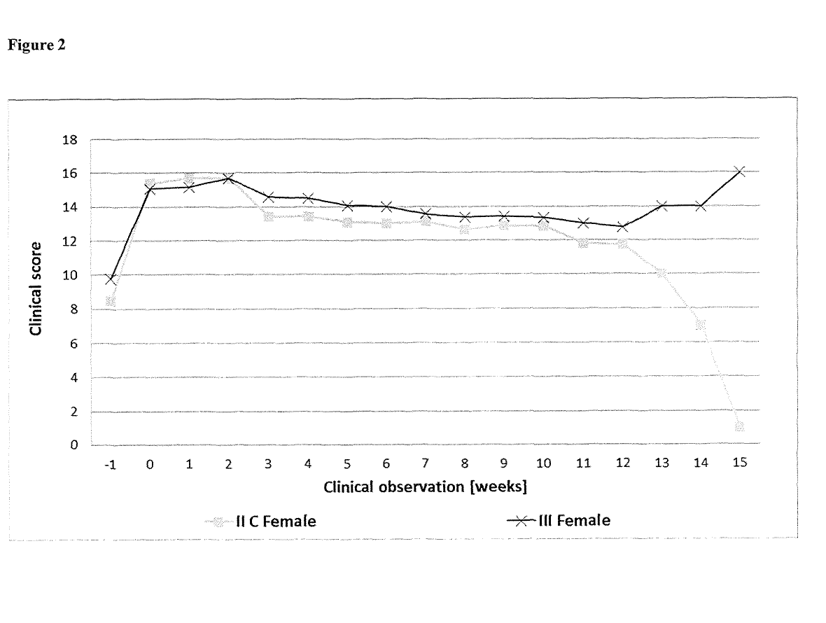

This analysis showed a reduction in the clinical symptoms of rheumatoid arthritis after treatment with the cell product of example 1 according to Table 1). The best results were obtained in female mice of Group IIC shown in FIG. 2.

Furthermore, the cells of the cell product obtained according to Example 8 were stained with a fluorescent dye prior to intravenous administration of the cell product to the mice (PKH26GL RED Sigma-Aldrich

https://www.sigmaaldrich.com/content/dam/sigma-aldrich/docs/Sigma/Bulletin/mini26bul.pdf). Almost every week between the 2^{nd} and 15^{th} week of the observation period two mice of each group were sacrificed and the distribution of the cells administered at time point zero into various organs of the treated mice was traced. The organs of the sacrificed mice were isolated, the tissue homogenized and the labelled cells identified by FACS. The results are shown in Table 2.1 listing the percentage of cells appearing in various organs during the observation period.

Importantly, this analysis demonstrated that the administered cell population comprising non hematopoietic stem and precursor cells did not migrate in any substantial amounts to the bone marrow from where it originated in the donor mice nor to any other of the analysed organs such as lymph nodes until the 12^{th} week after administration. This observation is in agreement with the therapeutic effect and supporting that after the systemic administration of the cell product some of the transferred non-hematopoietic progenitor, multipotent and pluripotent stem cells such as mesenchymal stem cells migrated to the inflamed joints, where they reduced the auto-immune reaction against the tissue of the joints, induced tissue repair thereby causing relief of the clinical symptoms. Also it is thought that the late appearance of a small number, in the order of less than 0.3% of cells, in the bone marrow in the 13^{th} week could correlate with the signaling to endogenous bone marrow mesenchymal cells to promote tissue repair. Furthermore, the appearance of a small number in the order of 0.5 to 2% of the transferred cells in the liver around the 12^{th} to 14^{th} weeks is in line with necessary additional repair activity by the transferred cells in the liver. It is known that 20-25% of patients with rheumatoid arthritis show abnormalities in laboratory tests of liver function, but these have no connection either with the occurrence of clinical symptoms or with the presence of typical morphological changes. The most common abnormality in the image morphology is non-specific reactive hepatitis with steatosis, observed in 60-70% of cases, rarely widening bays liver. Another frequently found abnormality is nodular regenerative hyperplasia (see e.g. Reynolds W. J., Wanless I. R.: Nodular regenerative hyperplasia of the liver in a patient with rheumatoid vasculitis, J. Rheumatol. 1984. 2: 838-42.)

TABLE 2

| | | week of observation | | | | | | | | | | | |
|---|---|---|---|---|---|---|---|---|---|---|---|---|---|
| ORGAN | GROUP | 2 | 4 | 5 | 6 | 8 | 9 | 10 | 11 | 12 | 13 | 14 | 15 |
| Bone Marrow | IIA | | 0 | | | 0.01 | | | | 0.01 | 0.03 | 0 | 0 |
| | IIB | | 0 | | | 0 | | | | 0 | 0.24 | 0 | 0.01 |
| | IIC | | 0 | | | 0.01 | | | | 0 | 0.18 | 0 | 0 |
| Kidneys | IIA | 0 | 0 | 0 | | 0 | 0 | 0 | 0 | 0.02 | 0.02 | 0 | 0 |
| | IIB | 0 | 0 | 0 | | 0.03 | 0 | 0 | 0 | 0.01 | 0.22 | 0 | 0 |
| | IIC | 0 | 0 | 0 | | 0 | 0 | 0 | 0 | 0.03 | 0 | 0 | 0 |
| Lymph nodes | IIA | | 0 | 0 | 0.01 | 0.01 | 0.1 | 0 | 0 | 0 | 0 | 0.08 | 0.02 |
| | IIB | | 0 | 0 | 0.01 | 0.02 | 0.03 | 0.02 | 0 | 0 | 0 | 0.06 | 0.01 |
| | IIC | | 0 | 0.06 | 0.01 | 0.02 | 0.06 | 0 | 0 | 0 | 0.01 | 0.05 | 0 |
| Liver | IIA | 0 | 0 | 0 | 0 | 0 | 0 | 0 | 0 | 0 | 0.04 | 1.82 | 0.02 |
| | IIB | 0 | 0 | 0 | 0 | 0.01 | 0 | 0.02 | 0.03 | 0.49 | 0 | 0.02 | 0.03 |
| | IIC | 0 | 0 | 0 | 0 | 0.04 | 0 | 0.02 | 0.01 | 0.01 | 0 | 1.98 | 0 |

### Example 3: Diabetes Type I

In Example 3, the therapeutic activity of the cell product prepared according to Example 1 was tested in vivo in the experimental model of type I diabetes in mice.

The in vivo diabetes type I model disease was induced by intraperitoneal injection of streptozotocin according to the following procedure:

Streptozotocin (STZ) at a dose of 40 mg/kg of body weight dissolved in citrate buffer (0.1 M, pH 4.5) was intraperitoneally injected to mice of both sexes of strain C57BL/6 at the age of 10-11 weeks for 5 consecutive days. The maximum volume of the injection was 200 μl. The solution was administered on an empty stomach with a 12-hour withdrawal of food, while the food was restored after injection. All animals throughout the induction period of the diabetes model had free access to water. (For further information see: Boone-Villa VD et al: Effect of Varying Dose and Administration of Streptozotocin on Blood Sugar in Male CD1 Mice; Proc West Pharmacol Soc. 2011;54:5-9.)

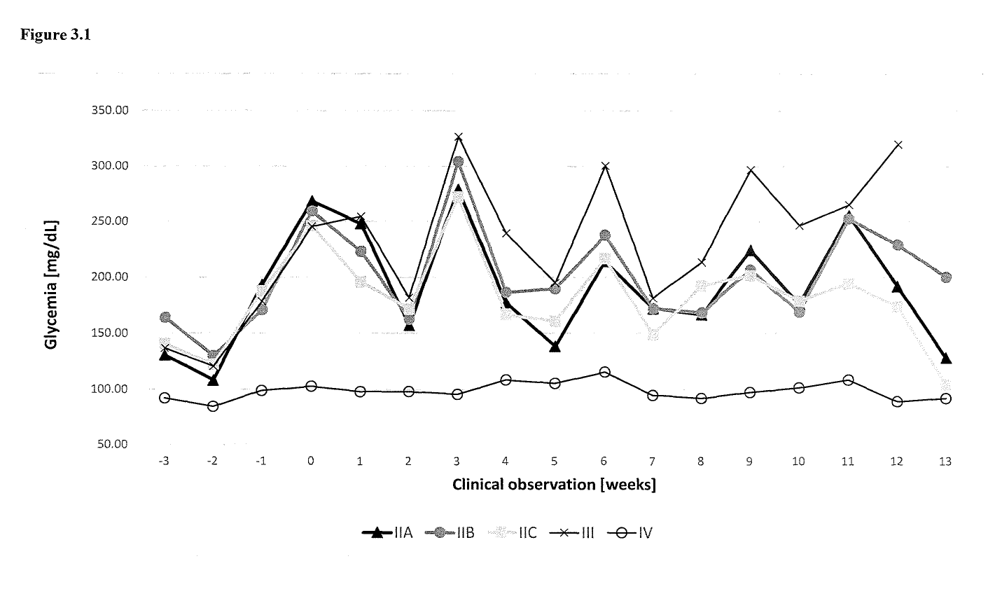

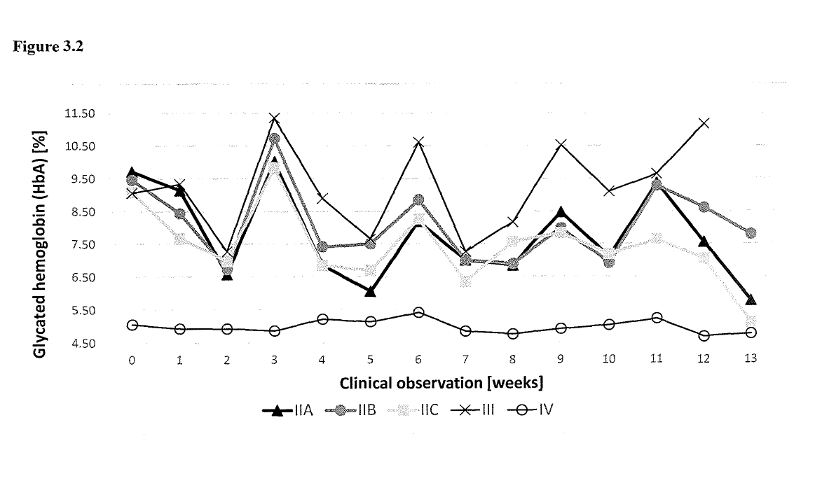

The stem cells comprising cell product was transferred to mice of 3 groups IIA, IIB and IIC according to Table 1 with various doses of transferred stem cell product. After the transfer blood values of the mice were evaluated once per week for glycemia and glycated haemoglobin (HbA) using standard test strips. The results of this analysis of both treated (IIA, IIB, IIC) and untreated (III) groups of mice with diabetes type I as well as a healthy control group (IV) are shown in FIGS. 3.1 and 3.2.

Hyperglycaemia develops primarily by direct cytotoxic action on the beta cells alpha and leaves delta cells intact and it is the result of an insulin deficiency rather than the consequence of an insulin resistance. It is noted that diabetes induced by chemicals such as stz is generally less stable and is reversible. Furthermore the administered chemical produces toxic actions on other body organs besides its cytotoxic action on beta cells. Therefore, the variability of results regarding hyperglycaemia is high. It was observed that the mice treated with stz displayed a depressed mental status with less activity besides typical symptoms associated with the diabetic state such as polyphagia, polydipsia and polyuria. The control mice displayed normal activity and were vital. They consumed water and food ad libitum and naturally gained weight.

These results shown in FIG. 3.1 demonstrate a decrease in glycemia in treated mice compared to untreated mice starting already in the first week after cell transfer, generally the decrease being the most pronounced in group IIC which received the highest number of cells (5×10⁶ cells per mouse) and results in approaching normal levels towards the end of the observation period of 13 weeks after administration of the cell product. In contrast, in untreated animals of group III blood glucose levels had approx. tripled in week 12 after which time point the last untreated mouse died.

As shown in FIG. 3.2, also the blood levels of glycated haemoglobin decreased starting about three weeks after the transfer of the cell product and approached normal levels for Group IIC in week 13, whereas in untreated animals of group III glycated haemoglobin levels is elevated approx. by a factor of two in week 12. Glycated hemoglobin (hemoglobin A1c, HbA1c, A1C, or Hb1c) is a form of haemoglobin (see also https://en.wikipedia.org/wiki/Glycated_hemoglobin. Normal levels of glucose result in a normal amount of glycated haemoglobin and an increase in the average amount of plasma glucose produces in a predictable increase of the fraction of glycated haemoglobin. The level of glycated haemoglobin serves as a marker for average blood glucose levels over the previous three months. In diabetes mellitus, higher amounts of glycated hemoglobin, indicate a poorer control of blood glucose levels associated with cardiovascular disease, nephropathy, neuropathy, and retinopathy.

The cells of the cell product were stained with a fluorescent dye prior to intravenous administration of the cell product to the mice and every week between the 1^{st} and 12^{th} week of the observation period two mice of each group were sacrificed and the distribution of the cells administered at time point zero into various organs of the treated mice was traced as described above for Example 2. The results are shown in Table 3 listing the percentage of cells appearing in various organs during the observation period.

After i.v. transfer, the passage of the labelled cells through all organs which are well supplied with blood was observed.

As can be seen, some labelled cells appear in the kidneys with increased levels during some weeks of the observation period. In chronic diabetes nephropathy can develop as a result of hyperglycemia due to high levels of blood sugar which causes damage to the glomeruli and kidney failure. This is one of the more severe complications of diabetes, which leads to high blood pressure, anemia and edemas. Stem cells may prevent or repair damage to the kidneys.

Transferred cells also migrate to the liver where increased levels are observed in various weeks of the observation period. As a result of the long-term course of diabetes, every person with diabetes experiences liver disorders due to the disturbance of the carbohydrate and the fat metabolism, manifested by excessive accumulation of both glycogen and fat in the liver. Both may lead to cirrhosis and steatosis and also to dysfunction of the gallbladder and bile ducts. Diseases of the liver and bile ducts occur with both type of diabetes (type I and type II). Furthermore, diabetes causes microvascular dysfunction which may also harm the liver which as the largest metabolizing organ is well supplied with blood vessels. Stem cells in the liver may prevent progressive damage to the liver.

Type I diabetes is known as an autoimmune disorder. The increased appearance of transferred cells in the lymph nodes at the beginning of the observation period supports a therapeutic effect of the stem cells by alleviating autoimmune reactions resulting in prevention of a progression of the diabetes by pancreatic islet regeneration and also protection of other organs against diabetes complications.

After intravenous transfer, homing of transferred cells to the bone marrow at the beginning of or intermittent during the observation period has been observed. The bone marrow is a reservoir of stem cells from where they can relocate to peripheral organs as needed for repair of damaged tissue.

TABLE 3
Cells administration after i.v. transfer in Diabetes Mellitus Type I

| | | week of observation | | | | | | | | | | | |
|---|---|---|---|---|---|---|---|---|---|---|---|---|---|
| ORGAN | GROUP | 1 | 2 | 3 | 4 | 5 | 6 | 7 | 8 | 9 | 10 | 11 | 12 |
| Kidneys | IIA | 0.26 | 0.04 | 0.13 | 0.05 | 0.02 | 0.02 | 0.19 | 0.33 | 0.55 | 0.07 | 0.03 | 0.25 |
| | IIB | 0.04 | 0.03 | 0.07 | 0.00 | 0.76 | 0.02 | 0.12 | 0.19 | 0.64 | 0.06 | 0.03 | 0.10 |
| | IIC | 0.44 | 0.00 | 0.05 | 0.00 | 0.00 | 0.12 | 0.10 | 0.09 | 0.07 | 0.05 | 0.04 | 0.10 |
| Spleen | IIA | 0.00 | 0.02 | | 0.00 | 0.00 | 0.00 | 0.07 | 0.06 | 0.02 | 0.13 | 0.04 | 0.19 |
| | IIB | 0.03 | 0.01 | | 0.00 | 0.00 | 0.01 | 0.04 | 0.11 | 0.03 | 0.11 | 0.06 | 0.20 |
| | IIC | 0.07 | 0.01 | | 0.00 | 0.3 | 0.01 | 0.01 | 0.02 | 0.01 | 0.25 | 0.01 | 0.08 |
| Liver | IIA | 0.00 | 0.23 | 0.75 | 0.71 | 0.05 | 0.18 | 2.26 | 0.00 | 0.86 | 12.55 | 0.03 | 0.01 |
| | IIB | 0.62 | 0.70 | 0.22 | 0.52 | 0.05 | 0.16 | 2.36 | 0.04 | 0.25 | 1.30 | 0.20 | 0.55 |
| | IIC | 0.23 | 0.20 | 0.68 | 0.10 | 0.04 | 0.16 | 0.80 | 0.05 | 0.08 | 1.07 | 0.01 | 0.29 |
| Lymph nodes | IIA | 4.61 | 2.99 | 7.37 | 1.75 | 11.44 | 4.30 | 0.20 | 0.03 | 1.77 | 1.70 | 0.24 | 0.06 |
| | IIB | 2.32 | 0.30 | 0.53 | 0.95 | 3.45 | 3.55 | 3.01 | 0.53 | 0.31 | 0.06 | 2.41 | 0.13 |
| | IIC | 2.39 | 2.31 | 8.17 | 4.28 | 1.74 | 23.74 | 0.70 | 0.04 | 0.11 | 0.03 | 0.11 | 0.12 |
| Bone marrow | IIA | 1.05 | 0.33 | | 0.14 | 0.21 | | 0.00 | 0.43 | 0.51 | | 0.02 | 0.01 |
| | IIB | 1.13 | 0.84 | | 1.27 | 0.12 | | 0.01 | 0.44 | 0.33 | 0.00 | 0.00 | 0.01 |
| | IIC | 1.11 | 0.81 | | 0.16 | 0.19 | | 0.03 | 0.29 | 0.28 | 0.02 | 0.01 | 0.02 |

### Example 4: Diabetes Type II

In Example 4, the therapeutic activity of the cell product prepared according to Example 1 was tested in vivo in the experimental model of type II diabetes in mice.

Induction of type II diabetes in a mouse model was performed by intraperitoneal injection of streptozotocin (STZ) dissolved in citrate buffer (0.05 M, pH 4.5) to mice of both sexes of strain C57BL/6 at the age of 10-11 weeks at a dose of 100 mg/kg body weight in two doses at an interval of 2 days. 15 minutes before the administration of the STZ solution, nicotinamide (NA) at a dose of 240 mg/kg body weight dissolved in physiological saline was intraperitoneally injected. The maximum volume of the injection was 200 μl. The solution was applied after 12-16 hours of food withdrawal, and after the injection, food was returned. Throughout the induction period of the diabetes type II model disease the mice had free access to water. (For further information see e.g. Nakamura T et al: Establishment and pathophysiological characterization of type 2 diabetic mouse model produced by streptozotocin and nicotinamide. Biol Pharm Bull. 2006; 29:1167-74.)

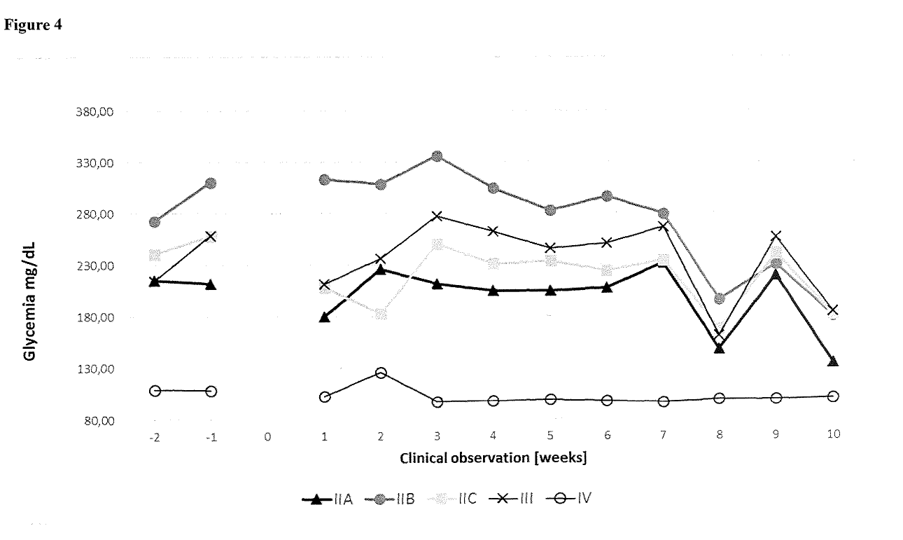

Stem cells were transferred to mice in various doses according to Table 1. The mice were examined with respect to glycemia, see FIG. 4, which shows the blood glucose levels in the female mice of treated (Groups IIA, IIB, IIC) and untreated (Group III) mice with diabetes type II compared to healthy control mice (Group IV). As can be seen, treatment with the stem cell product results generally in a lowering of glycemia when compared to untreated mice. It should be noted that in the case of the female treated mice of group IIB, the therapeutic effect is most pronounced—see the difference in the level of blood glucose at the beginning and at the end of the clinical observation period.

Furthermore, the administered cells were stained with a fluorescent dye prior to intravenous administration of the cell product to the mice and every week between the 1^{st} and 10^{th} week of the observation period two mice of each group were sacrificed and the distribution of the cells administered at time point zero into various organs of the treated mice was traced as described above for Example 2. The results are shown in Table 3 listing the percentage of cells appearing in n various organs during the observation period.

As discussed above for diabetes type I, the peripheral appearance of the labelled cells in various organs represents the circulation of the cells after i.v. transfer through all organs which are well supplied with blood e.g. to the liver and kidneys. They stem cells can provide a therapeutic effect by immunosuppression or provide repair of tissue damaged due to diabetes e.g. in the liver or kidneys. Again the homing and releasing of labelled cells is observed and appearance of cells in the pancreas where they may effect pancreatic islet regeneration for the production of the physiologically active form of insulin.

TABLE 4
Cells administration after i.v. transfer in Diabetes Mellitus Type II (% of cells)

| | | week of observation | | | | | | | | | |
|---|---|---|---|---|---|---|---|---|---|---|---|
| Organ | Group | 1 | 2 | 3 | 4 | 5 | 6 | 7 | 8 | 9 | 10 |
| Kidneys | IIA | 0.02 | 0 | 1.65 | 0.07 | 0.02 | 0.02 | 0.01 | 0.01 | 0.04 | 0.01 |
| | IIB | 0.01 | 0.1 | 0.59 | 0.04 | 0 | 0.03 | 0.01 | 0 | 0.05 | 0.02 |
| | IIC | 0.02 | 0 | 1.62 | 0.03 | 0.01 | 0.03 | 0.01 | 0 | 0.06 | 0.04 |
| Spleen | IIA | 0 | 0.02 | 0.01 | 0.02 | 0.01 | 0.02 | 0 | 0.01 | 0.02 | 0.03 |
| | IIB | 0.02 | 0.03 | 0.03 | 0.01 | 0 | 0.01 | 0 | 0 | 0 | 0.04 |
| | IIC | 0.01 | 0.02 | 0.02 | 0 | 0 | 0.02 | 0 | 0.01 | 0 | 0.01 |
| Liver | IIA | 0.01 | 0.04 | 0.33 | 0.13 | 0 | 0.1 | 0.01 | 0.1 | 0.03 | 0.15 |
| | IIB | 0.02 | 0.03 | 0.28 | 0.04 | 0.01 | 0.09 | 0.01 | 0.06 | 0.02 | 0.15 |
| | IIC | 0.01 | 0.03 | 0.11 | 0.12 | 0.01 | 0.1 | 0 | 0.04 | 0.03 | 0.2 |
| Lymph nodes | IIA | 0 | 0.14 | 0.23 | 0.11 | 0.01 | 0.1 | 0.06 | 0.01 | 0.03 | 0.1 |
| | IIB | 0 | 0.02 | 0.06 | 0 | 0 | 0.17 | 0.06 | 0.03 | 0.04 | 0.16 |
| | IIC | 0 | 0.02 | 0.16 | 0.1 | 0.01 | 0.08 | 0.03 | 0.01 | 0.04 | 0.16 |
| Bone marrow | IIA | | | 0.03 | 0 | 0 | 0 | 0.05 | 0 | 0.01 | 0.01 |
| | IIB | | | 0.01 | 0 | 0.01 | 0.05 | 0.02 | 0.01 | 0.01 | 0.01 |
| | IIC | | | 0.2 | 0 | 0.01 | 0 | 0.02 | 0 | 0 | 0.01 |
| Pancreas | IIA | 0 | 0 | 0 | 0.03 | 1.16 | 0.03 | 0.01 | 0.11 | 0.03 | 0 |
| | IIB | 0 | 0 | 0.3 | 0.05 | 0.26 | 0.03 | 0 | 1.56 | 0.07 | 0 |
| | IIC | 0 | 0.01 | 0.4 | 0 | 0.2 | 0.41 | 0.01 | 0.07 | 0.04 | 0 |

### Example 5: Ischemic Stroke

In Example 5, the therapeutic activity of the cell product prepared according to Example 1 was tested in vivo in a mouse model of ischemic stroke induced by photothrombosis in vessels in the cerebral cortex of mice under conditions according to the established procedure as described by Brant D. Watson et al., Ann Neurol 17: 497-504, 1985:

40 male and 40 female, 11-16 weeks old, C57BL/6 mice were subjected to surgery and photothromobosis causing ischemic stroke as described below. In addition, 20 mice of both sexes were subjected to sham surgery. Surgery was performed under anaesthesia by inhalation of isoflurane in oxygen at appropriate concentrations (3-5%).

Then, each mouse was placed in a stereotactic apparatus, the skull exposed through an incision in the midline of the skin and the periosteum was dissected approx. 2 mm from the bregma (stereotactic atlas of Franklin and Paxinos). Next a sterile fresh solution of

Rose Bengal dissolved in saline was administered intravenously (tail vein) at a dose exceeding 100 mg/kg body weight. Then the previously prepared surface of the skull was illuminated with green laser light (Infinity 0.5 H532L-G50B), males for 60 seconds, females for 45 seconds. After the exposure the wound was closed. These mice were divided into groups II and III according to Table 1.

In the sham surgery procedure 10 mice in control group IV were given Rose Bengal and not subjected to irradiation and 10 mice in control group IV were exposed to radiation without administration of the dye.

Mice of Groups IIA to IIC according to Table 1 were treated by intravenous administration of cell product obtained according to Example 1 from bone marrow of mice of the same strain C57BL/6 which also were subjected to surgery for induction of an ischemic stroke at the 0-time point.

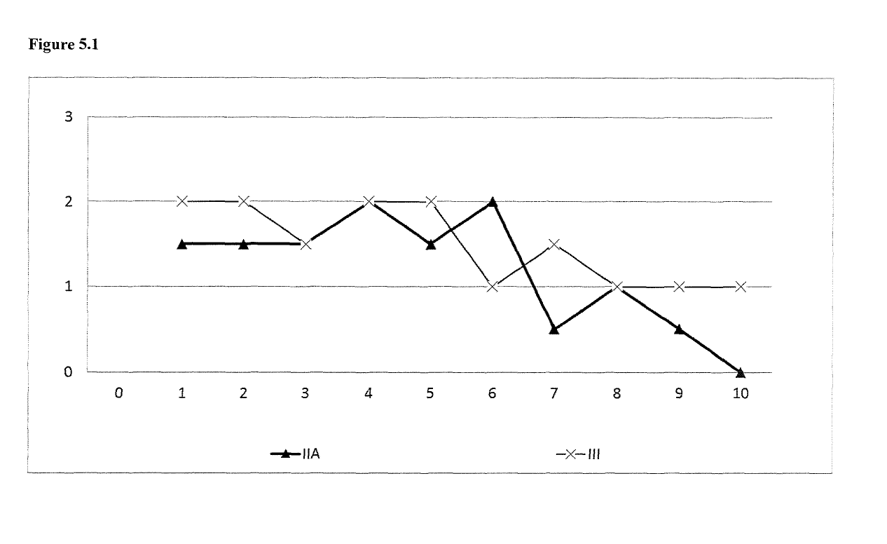

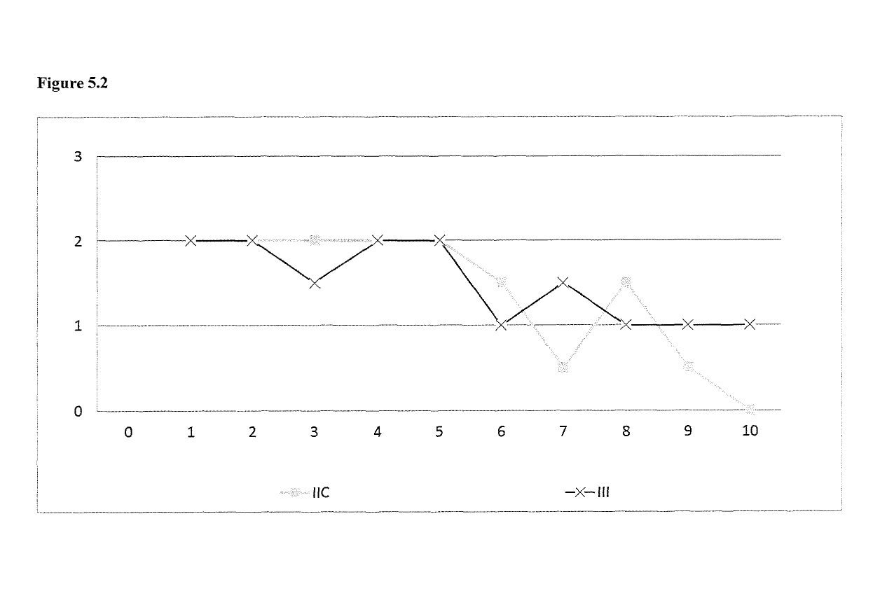

Each animal was clinically evaluated once a week subsequent to surgery over a period of 10 weeks as shown in FIGS. 5.1 and 5.2. A modified five-point scale was applied (H. Hara et al., Journal of Cerebral Blood Flow and Metabolism 1996 16: 605-611), where the values 1 to 5 correspond to the following clinical symptoms:
0—normal motility, no altered neurological signals
1—bending body and curl feet when lifting the tail (mild stroke)
2—circulation to one side of the tilt of the body, but normal posture at rest (moderate impact)
3—inclination of the body at rest (moderately severe stroke)
4—lack of spontaneous activity (severe stroke)
5—death

FIG. 5.1 and FIG. 5.2 show the results of analysis of neurological deficits according to the above criteria in both treated groups IIA and IIC, respectively, in comparison with untreated group III.

As can be seen the neurological symptoms of mice of both of the treated groups IIA and IIC decrease 2s from level between 1.5 and 2 corresponding to a moderate impact at the beginning to level of one or below one around week 8 or 9 and even to a level of zero corresponding to normal motility without altered neurological signals in the 10^{th} week of observation.

Furthermore, from the 3^{rd} to the 10^{th} week of the observation period one male and one female mouse from each group, was sacrificed and the distribution of the cells administered at time point zero into various organs of the treated mice was traced.

Tables 5.1 and 5.2 show the tracing results of the fluorescently labelled cells after administration of the cell product similarly as described above in example 2 where Table 5.1 shows the results for the male mice and Table 5.2 for the female mice.

Interestingly, this analysis demonstrated that the a very small percentage of less than 2% in males and no more than 3.1% in females of the administered cell population comprising non hematopoietic stem and precursor cells appeared in the bone marrow from where it originated in the donor mice, whereas after week 8 essentially none of the labelled cells (less than 0.1%) are found in the bone marrow. This observation is suggestive of some of the administered cells migrating initially to the bone marrow where it induces the production of further stem and progenitor cells in the bone marrow which promote the regeneration of damaged brain tissue. Similarly a small percentage of administered cells appears in the spleen between weeks 5 and 7 suggesting clearance of the cells from the circulation.

TABLE 5.1

| Male | | week of observation | | | | | | | |
|---|---|---|---|---|---|---|---|---|---|
| ORGAN | GROUP | 3 | 4 | 5 | 6 | 7 | 8 | 9 | 10 |
| Bone marrow | IIA | 0.65 | 2.32 | 0.63 | 0.88 | 1.71 | 0.03 | 0.04 | 0.01 |
| | IIB | 0.41 | 1.12 | 1.7 | 0.43 | 1.19 | 0.03 | 0.02 | 0 |
| | IIC | 0.24 | 0.64 | 1.76 | 0.52 | 0.98 | 0.06 | 0.03 | |
| Kidneys | IIA | 0.02 | 0.05 | 0.02 | 0.01 | 0.01 | 0.02 | 0 | 0.02 |
| | IIB | 0.04 | 0.02 | 0.03 | 0.1 | 0.03 | 0.03 | 0.02 | 0.01 |
| | IIC | 0.09 | 0.02 | 0.04 | 0.02 | 0.02 | 0.01 | 0.05 | |
| Spleen | IIA | 0.02 | 1.33 | 0.64 | 2.3 | 0.78 | 0.01 | 0.05 | 0 |
| | IIB | 0.03 | 0.81 | 1.07 | 0.81 | 0.28 | 0 | 0.02 | 0 |
| | IIC | 0.09 | 1.15 | 2.18 | 0.74 | 0.26 | 0.04 | 0.24 | |
| Lymph nodes | IIA | 0.05 | 0.07 | 0.11 | 0.21 | 0.07 | 0.02 | 0 | 0 |
| | IIB | 0.1 | 0.04 | 1.7 | 0.23 | 0.11 | 0.02 | 0.01 | 0.02 |
| | IIC | 0.12 | 0.08 | 0.07 | 0.36 | 0.22 | 0.01 | 0 | |
| Liver | IIA | 0.76 | 0.35 | 0.66 | 0.09 | 1.65 | 0 | | 0.02 |
| | IIB | 0.47 | 0.27 | 0.73 | 0.05 | 2.17 | 0 | 0.01 | 0.06 |
| | IIC | 1.02 | 0.93 | 2.34 | 0.22 | 3.71 | 0.3 | 0.51 | |
| Pancreas | IIA | 1.09 | 0.49 | 0.27 | 0.02 | 1.36 | | 0 | 0 |
| | IIB | 1 | 0.25 | 0.37 | 0.12 | 0.27 | | 0.01 | 0 |
| | IIC | 0.41 | 0.28 | 0.64 | 0.08 | 0.1 | | 0.01 | |

TABLE 5.2

| Female | | week of observation | | | | | | | |
|---|---|---|---|---|---|---|---|---|---|
| ORGAN | GROUP | 3 | 4 | 5 | 6 | 7 | 8 | 9 | 10 |
| Bone marrow | IIA | 0.41 | 0.34 | 1.89 | 0.66 | 0.74 | 0.02 | 0.05 | |
| | IIB | 0.7 | 0.4 | 3.08 | 0.39 | 1.04 | 0.02 | 0.02 | |
| | IIC | 0.55 | 0.36 | 1.23 | 0.58 | 1.16 | 0.01 | 0.08 | 0.01 |
| Kidneys | IIA | 0 | 0 | 0.08 | 0.01 | 0 | 0.02 | 0.01 | |
| | IIB | 0.04 | 0.03 | 0.02 | 0.01 | 0.01 | 0.05 | 0 | |
| | IIC | 0 | 0.01 | 0.07 | 0.04 | 0.01 | 0.01 | 0 | 0 |
| Spleen | IIA | 0.01 | 1.03 | 2.81 | 1.72 | 0.12 | 0 | 0.01 | |
| | IIB | 0.04 | 1.23 | 1.71 | 1.48 | 0.21 | 0 | 0.01 | |
| | IIC | 0.08 | 0.75 | 1.51 | 1.15 | 0.47 | 0 | 0.01 | 0.02 |
| Lymph nodes | IIA | 0.04 | 0.1 | 0.04 | 0.17 | 0.12 | 0.02 | 0.01 | |
| | IIB | 0.03 | 0.05 | 1.57 | 0.13 | 0.14 | 0.03 | 0 | |
| | IIC | 0.07 | 0.08 | 0.13 | 0.17 | 0.13 | 0.01 | 0.02 | 0.01 |
| Liver | IIA | 0.48 | 0.17 | 2.98 | 0.06 | 0.73 | 0.03 | 0.08 | |
| | IIB | 0.39 | 0.31 | 1.49 | 0.05 | 0.38 | 0.01 | 0.06 | |
| | IIC | 1.12 | 1.3 | 1.83 | 0.21 | 0.95 | 0.12 | 0.23 | 0.21 |
| Pancreas | IIA | 2.42 | 0.68 | 0.48 | 0.16 | 0.09 | | 0 | |
| | IIB | 1.98 | 0.78 | 0.51 | 0.13 | 0.1 | | 0 | |
| | IIC | 0.47 | 0.99 | 0.68 | 0.1 | 0.08 | | 0 | 0.01 |

For further information see e.g.

Brant D. Watson, W. Dalton Dietrich, Raul Busto, Mitchell S. Wachtel, Myron D. Ginsberg: Introduction of reproducible brain infarction by photochemically initiated thrombosis. Ann Neurol 17: 497-504, 1985

H. Hara, P. L. Huang, N. Panahian, M. C. Fishman, M. A Moskowitz: Reduced brain edema and infarction volume in mice lacking the neuronal isiform of nitric oxide synthase after transient MCA occlusion. Journal of Cerebral Blood Flow and Metabolism 16:605-611

L. Zeng, X. He, J. Liu, L. Wang, S. Weng, Y. Wang, S. Chen, G.-Y. Yang: Differences of circulating inflammatory markers between large and small vessel disease in patient with acute ischemic stroke. Int. J. Med. Sci. 2013, Vol. 10 (10):1399-1405.

### Example 6: Myocardial Infarction

In Example 6, the therapeutic activity of the cell product prepared according to Example 1 was tested in vivo in the experimental model of myocardial infarction in mice as described in Kumar V. et al., Indian J. Exp. Biol. 2009 Sep;47(9):730-6 and Brooks W. et al., Comp. Med. 2009; 59(4): 339-343.

The experimental model of myocardial infarction was induced in 354 mice of both sexes BALB/c to at the age of 10-11 weeks by a single subcutaneous administration of isoproterenol(Sigma) at a dose of 250 mg/kg body weight dissolved in sterile 0.9% NaCl.

Isoproterenol, 4-{1-hydroxy-2-[(propan-2-yl) amino] ethyl) benzene-1,2-diol, used in the experiments is a non-selective agonist of β-adrenergic receptors. This compound causes increased heart rate (tachycardia), promotes arrhythmias, and strengthens cardiac contraction.

The cardiotoxicity of isoproterenol resulted primarily from:
hypoxia and ischemia
coronary insufficiency
metabolic disorders
exhaustion of intracellular ATP stores
electrolyte disturbances
ion pump function impaired cells and accumulation of Ca²⁺
oxidative stress
disorders of homeostasis
damage to the intracellular structures

For testing the therapeutic activity of the cell product obtained according to Example 1, the cell product was administered to mice of Groups II A-C according to Table 1 approx. four weeks after the isoproterenol injection. The control group III of diseased mice was not administered the cell product. The healthy control group IV was injected with the carrier 0.9% NaCl, only.

Once every two weeks one randomly selected each of the male and female animals from each test group were bled for diagnostic purposes and for obtaining cardiac tissue homogenates. The results are shown in Tables 6.1 and 6.2 further below.

Once every four weeks (or in weeks 4, 12 and 16) the number of animals was doubled (2 males, 2 females) for obtaining additional heart tissue which was stained with 2,3,5-trifenylotetrazolowym (TTC) and served the analysis of the extent of myocardial necrosis (Conci E. et al. Mouse Models for Myocardial. Ischaemia/Reperfusion. J. Cardiology 2006; 13 (7-8), 239-244) by examination of the images obtained after staining the histological sections using Masson's TTC staining protocol.

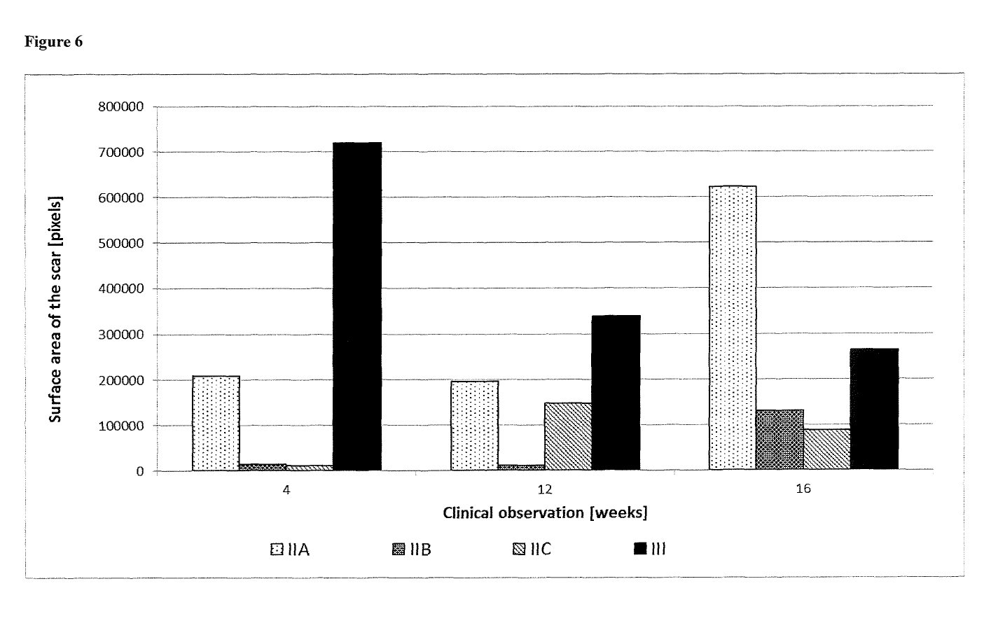

The effect of intravenous transfer of the stem cell product obtained according to Example 1 on the post-infarction heart scar of males in the above described mouse model of myocardial infarction is shown in FIG. 6 for weeks 4, 12 and 16 after transfer of the cell product as indicated on the x-axis. The hearts obtained in week 8 showed no signs of fibrosis, thus these hearts were not stained for collagen. The bars in the graph represent the content of collagen in the heart based on the analysis of images obtained after staining the histological sections using Masson's trichrome in selected animals in 4, 12 and 16 week clinical observation.

As can be seen, the surface area of the scar as indicated in pixels is the largest in the hearts of untreated mice of Group III and the area decreases with time as seen at weeks 12 and 16. The scar size of the treated animals of Groups IIA to IIC is smaller. However, these results have to be interpreted with precaution, because the scar size should not increase with time, which is what was observed in particular for Group IIA at the 16 week examination. This points to some difficulties inherent with this test such as individual variability of the size of the heart and the individual susceptibility to the amount of tissue damage induced by an infarction.

Tables 6.1 and 6.2 below show the tracing results of the fluorescently labelled cells in the administered cell product over a period of 18 weeks starting with the week when the cell product was administered at the 0-time point into various organs of the treated mice. After the intravenous administration of the cell product obtained according to Example 1 slow progressive accumulation of labelled cells in bone marrow, kidney, lymph nodes and liver is observed. Some of the administered cells migrating initially to the bone marrow induces the production of further stem and progenitor cells in the bone marrow which promote the regeneration of damaged heart tissue. It is also suggested that the whole body distribution results in the release of immunological factors.

TABLE 6.1

| | | Week after transfer of cells | | | | | | | | | |
|---|---|---|---|---|---|---|---|---|---|---|---|
| | | with positive fluorescent signal [%] | | | | | | | | | |
| MALE | | 0 | 2 | 4 | 6 | 8 | 10 | 12 | 14 | 16 | 18 |
| ORGAN | GROUP | ♂ ♀ | ♂ ♀ | ♂ ♀ | ♀ | ♀ | ♀ | ♀ | ♀ | ♀ | ♀ |

| | | | | | | | | | | |
|---|---|---|---|---|---|---|---|---|---|---|
| Bone marrow | IIA | 0.02 | 0.09 | 0.76 | 0.37 | 0.12 | 2.02 | 0.87 | 0.6 | 0.71 |
| | IIB | 0.03 | 0.06 | 0.55 | 0.46 | 0.1 | 1.89 | 0.52 | 0.69 | |
| | IIC | 0.02 | 0.05 | 0.6 | 0.34 | 0.09 | 1.3 | 0.53 | 0.82 | |
| Kidneys | IIA | 0.05 | 0.06 | 0 | 0.14 | 0.19 | 0.22 | 0.24 | 0.04 | 0.02 |
| | IIB | 0.64 | 0.08 | 0 | 0.09 | 0.11 | 0.17 | 0.43 | 0.01 | |
| | IIC | 0 | 0.19 | 0.01 | 0.1 | 0.39 | 0.21 | 0.23 | 0.02 | |
| Spleen | IIA | 0.16 | 0.04 | 0.17 | 0.48 | 0.02 | 1.29 | 1.78 | 0.67 | 0.17 |
| | IIB | 0.17 | 0.05 | 0.24 | 0.66 | 0.04 | 3.29 | 1.64 | 0.41 | |
| | IIC | 0.12 | 0.05 | 0.18 | 0.63 | 0.03 | 2.07 | 1.14 | 0.59 | |
| Lymph nodes | IIA | 0.04 | 0.15 | 0.11 | 0.12 | 0.25 | 1.33 | 0.34 | 0.09 | 2.66 |
| | IIB | 0.19 | 0.18 | 0.09 | 0.45 | 0.09 | 1.18 | 0.41 | 0.26 | |
| | IIC | 0.79 | 0.51 | 0.05 | 0.29 | 0.17 | 0.34 | 0.17 | 0.12 | |
| Liver | IIA | 0.81 | 0.04 | 0.08 | 0 | 0.02 | 0.07 | 0.02 | 0.1 | 0.38 |
| | IIB | 0.94 | 0.11 | 0.02 | 0.01 | 0.03 | 0.06 | 0.09 | 2.88 | |
| | IIC | 1.17 | 0.03 | 0.04 | 0.01 | 0.02 | 0.05 | 0 | 0.94 | |

TABLE 6.2

| | | Week after transfer of cells | | | | | | | | | |
|---|---|---|---|---|---|---|---|---|---|---|---|
| | | with positive fluorescent signal [%] | | | | | | | | | |
| FEMALE | | 0 | 2 | 4 | 6 | 8 | 10 | 12 | 14 | 16 | 18 |
| ORGAN | GROUP | ♀ ♂ | ♀ ♂ | ♀ ♂ | ♂ | ♂ | ♂ | ♂ | ♂ | ♂ | ♂ |

| | | | | | | | | | | |
|---|---|---|---|---|---|---|---|---|---|---|
| Bone marrow | IIA | 0.02 | 0.08 | 0.3 | 0.53 | 0.03 | 3.3 | 0.61 | 0.51 | 0.29 |
| | IIB | 0.03 | 0.1 | 0.33 | 0.47 | 0.05 | 3.04 | 0.63 | 0.48 | 0.42 |
| | IIC | 0.02 | 0.06 | 0.4 | 0.4 | 0.07 | 2.5 | 0.55 | 0.73 | 0.41 |
| Kidneys | IIA | 0.05 | 0.06 | 0 | 0 | 0.46 | 0.26 | 0.15 | 0.03 | 0 |
| | IIB | 0.64 | 0.08 | 0 | 0 | 0.42 | 0.59 | 0.36 | 0.04 | 0.02 |
| | IIC | 0 | 0.19 | 0 | 0.01 | 0.5 | 0.16 | 0.09 | 0.04 | 0.02 |
| Spleen | IIA | 0.16 | 0.04 | 0.13 | 0.62 | 0.05 | 1.44 | 1.89 | 0.81 | 0.18 |
| | IIB | 0.17 | 0.05 | 0.09 | 0.57 | 0.01 | 1.31 | 1.77 | 1.88 | 0.11 |
| | IIC | 0.12 | 0.05 | 0.1 | 0.64 | 0 | 1.57 | 1.15 | 0.72 | 0.08 |
| Lymph nodes | IIA | 0.04 | 0.15 | 0.13 | 0.39 | 0.01 | 0.16 | 0.07 | 0.05 | 1.56 |
| | IIB | 0.19 | 0.18 | 0.12 | 0.16 | 0 | 0.08 | 0.2 | 0.07 | 0.46 |
| | IIC | 0.79 | 0.51 | 0.06 | 0.17 | 0.2 | 0.18 | 0.43 | 0.07 | 0.51 |
| Liver | IIA | 0.81 | 0.04 | 0.06 | 0 | 0 | 0.01 | 0.04 | 0 | 0.19 |
| | IIB | 0.94 | 0.11 | 0.06 | 0.01 | 0 | 0.04 | 0.31 | 0.02 | 0.17 |
| | IIC | 1.17 | 0.03 | 0.07 | 0.01 | 0 | 0.04 | 0.14 | 0 | 0.44 |

On the basis of these results obtained in mice, the following conclusions can be drawn:

The use of stem cell therapy in the treatment of myocardial infarction in a mouse model of the disease 1. is safe; 2. resulted in most cases of animals treated n with the cell product in inhibition of scar formation in favour of post-infarction repair of the scarred heart tissue; 3. resulted in most of the treated animals in a reversal of abnormal tissue morphology by growth of normal tissue.

### Example 7: Multiple Sclerosis

In Example 7, the therapeutic activity of the cell product prepared according to Example 1 as well as the original population of cells and the depleted hematopoietic cells were tested in vivo in an experimental model of multiple sclerosis (MS) in mice.

The best available animal model for MS, a progressive disease of the central nervous system with autoimmune aetiology, is the well-known model of experimental autoimmune encephalomyelitis (EAE) in mice. EAE is very similar to the MS with respect to the pathological changes in the CNS, as well as the clinical symptoms.

EAE was induced by immunization with myelin of female SJL mice/J (Jackson Laboratory, USA) obtained from the Department of Animal Breeding Experimental Medical University of Lodz at age 6-8 weeks. Mice were administered subcutaneously at two sites in the abdominal region the immunogenic peptide fragment PLP 139-151 mixed with complete Freud's adjuvant (CFA, Sigma). Each mouse was administered 0.25 ml of a suspension of a mixture of 15 mg of PLP peptide 139-151 dissolved in 0.1 ml of double distilled water and 0.75 mg of freeze-dried Mycobacterium tuberculosis H37Rv (Difco Lab., USA) suspended in 0.15 ml of CFA. In addition mice were administered to the tail vein 0.15 pg pertussis toxin (Pertussis toxin from Bordetella pertussis, Sigma) dissolved in physiological saline (Phosphate Buffered Saline—PBS, Biomed) to a final volume of 0.2 ml twice, on the day of immunization and on the third day after immunization.

Clinical observation was carried out every day at fixed times for neurological symptoms of EAE. The scale of assessment takes into account the motor skills and physical coordination of animals and permits the identification of neurological differences observed between the groups of animals. A scale with a total of six-grades according to published criteria (Pettinelli et al., 1982; Głąbiński et al., 1997) was used, where:
5—a disease of the nipple mortal;
4—paralysis of fore and hind limbs;
3—total paralysis of the hind legs or the front;
2—paresis or ataxia;
1—tail weakness;
0—no symptoms.

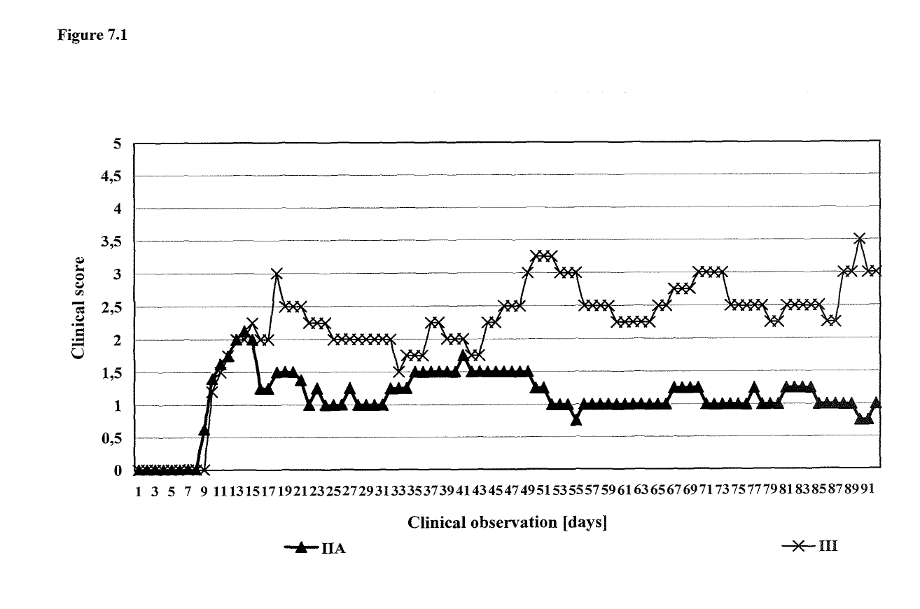

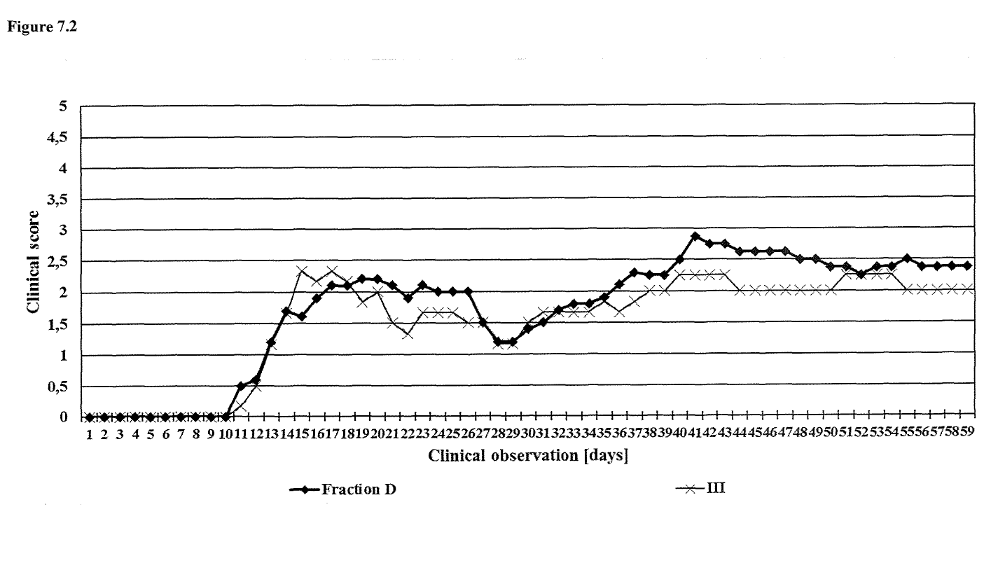

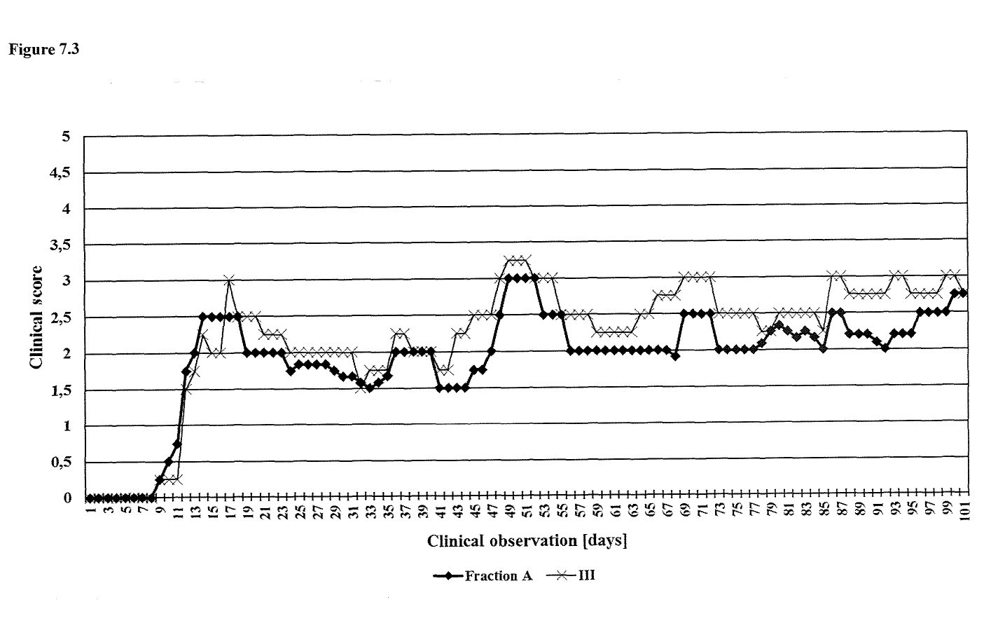

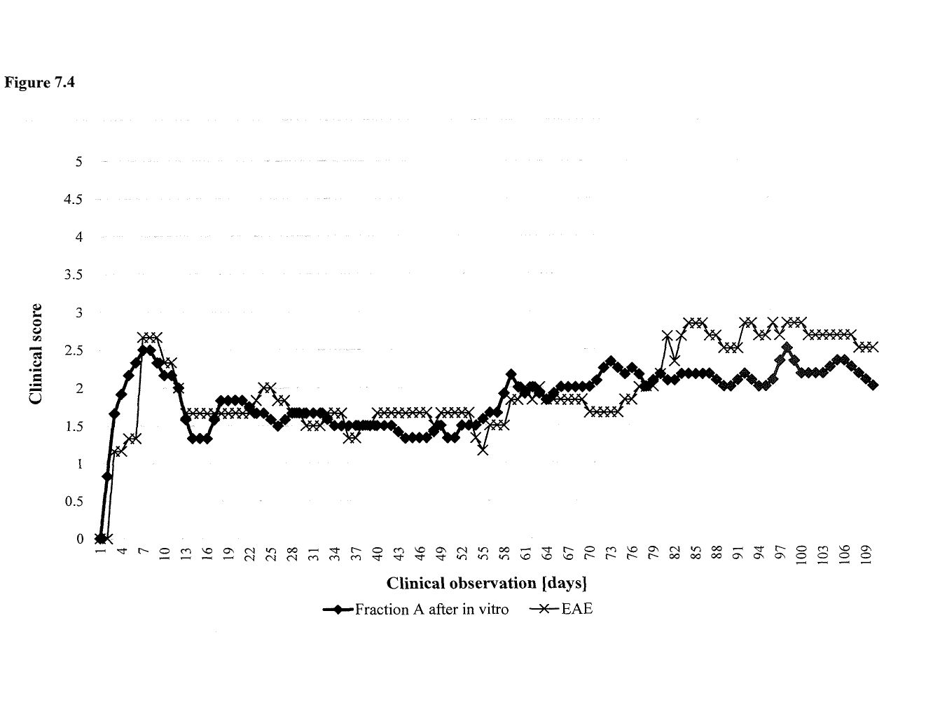

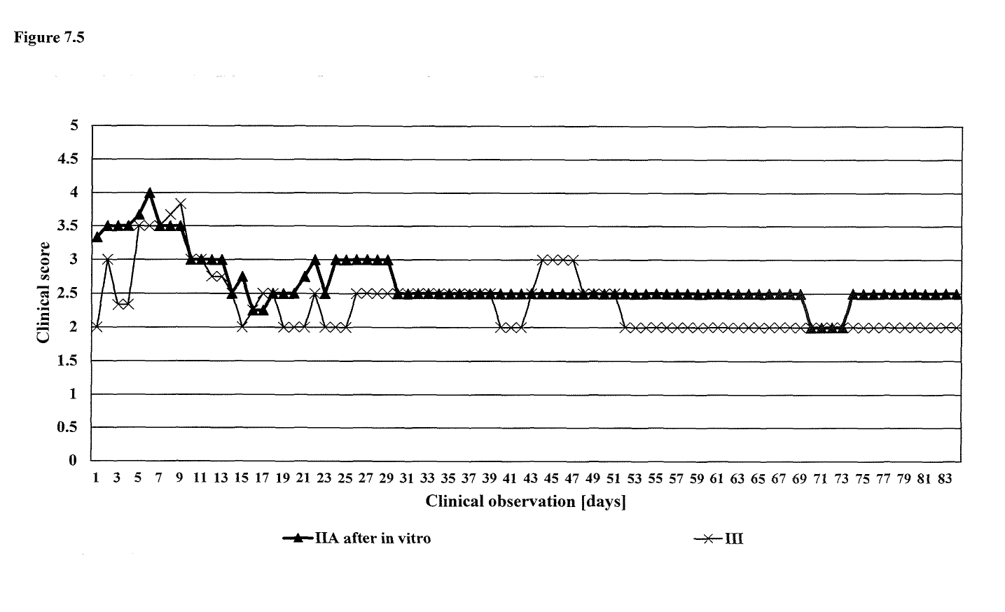

Common clinical symptoms of ERE typically appeared between day 10 and 15 after immunization. Treatment of EAE mice was performed by a single intravenous transfer of stem cells at a dose of 2×10e6 cells at the time of the first peak of disease EAE between day 10 and 15 after immunization. In Example 7 the therapeutic activity was tested not only of the freshly obtained fraction C (i.e. the exemplary inventive cell product which is depleted of hematopoietic cells, FIG. 7.1) but also of freshly obtained fraction D (fraction comprising the selected hematopoietic cells retained by the depletion column and subsequently eluted, FIG. 7.2) and freshly obtained fraction A (whole bone marrow, original population of cells, see FIG. 7.3) were tested.

In addition, the therapeutic activity of fraction A (see FIG. 7.4) and fraction C (see FIG. 7.5) was also tested after three weeks of in vitro cultivation, wherein in a 1^{st} step two weeks of proliferative culture conditions (culture medium supplemented with FGF2, EGF: DMEM/F12 (Gibco, Cergy, France) with: 0.6% glucose, 25 ug/ml insulin, 100 ug/ml transferrin, 20 nM progesterone, 60 mg/ml putrescine, 30 nM sodium selenite, 2 mM glutamine, 3 mM sodium bicarbonate, 5 mM HEPES, 2 mg/ml heparin, 50 mg/ml gentamicin, 20 ng/ml FGF2 and 20 ng/ml EGF) and in a 2^{nd} step one week of differentiation culture conditions (culture medium without FGF2, EGF: DMEM/F12 (Gibco, Cergy, France) with: 0.6% glucose, 25 ug/ml insulin, 100 ug/ml transferrin, 20 nM progesterone, 60 mg/ml putrescine, 30 nM sodium selenite, 2 mM glutamine, 3 mM sodium bicarbonate, 5 mM HEPES, 2 mg/ml heparin, 50 mg/ml gentamicin) were applied.

All of these different cell populations were administered to 10 EAE mice at the time of the first peak of EAE symptoms between day 10 and day 15 after immunization (FIG. 7.2 to FIG. 7.5) except for the fresh fraction C which was administered to 18 EAE mice (FIG. 7.1).

These results show very clearly that the cell product depleted of hematopoietic cells, fraction C according to Example 1, which was administered directly after the in vitro depletion procedure to 18 mice is therapeutically active, as the clinical symptoms measured by the scale indicated above decreased from a value around 2 to a value just below 1 by the end of the observation period in treated mice, where the relief of symptoms starts approx. with day 3 after administration of the cell product—corresponding to approx. 13 to 18 days after immunization (i.e. induction of EAE) of the mice. This relief of symptoms continues over the whole observation period up of 92 days after induction of EAE by immunization. In contrast, untreated mice rather show an marked increase in EAE symptoms up to a value of 3 within the 92-day observation period.

Furthermore, the EAE model disease in mice similarly to multiple sclerosis in humans progresses with a relapsing and remitting pattern of symptoms. As can be seen in FIG. 7.1 in the group of treated mice only one slight relapse of low intensity was observed, whereas in the control group three relapses of larger intensities are observed. This indicates an alleviation of the course of EAE disease in the treated group during the entire period of observation believed to be primarily due to the immunomodulatory actions of the intravenously transferred stem cell fraction C.

Strikingly, the beneficial therapeutic effect was only achieved by transfer of stem cell fraction C, the cell product obtained according to the exemplary embodiment of the method according to Example 1. No beneficial therapeutic effect was achieved either by whole bone marrow (fraction A, FIG. 7.3) or by the hematopoietic cells (fraction D, FIG. 7.2) or after three weeks of in vitro cultivation of fraction A (FIG. 7.4) or after three weeks of in vitro cultivation of fraction C (FIG. 7.5) under in cultivation conditions comprising such which favour differentiation. In all of FIGS. 7.2 to 7.5 the values measuring the clinical EAE symptoms do not significantly differ between treated and untreated mice.

Table 7 shows the tracing results of the fluorescently labelled cells in the administered cell product at weeks 1, 2 and 6 starting with the week when the cell product was administered.

TABLE 7

| | Week of observation | | |
|---|---|---|---|
| | 1 | 2 | 6 |
| Organ | ♀ | ♀ | ♀ |
| Brain | 0.14 | 0.05 | 0.37 |
| Brainstem + Medulla | 0.10 | 0.60 | 0.32 |
| oblongata | | | |
| Spinal cord | 0.16 | 0.80 | 0.82 |
| (upper section) | | | |
| Spinal cord | 2.76 | 1.40 | 0.83 |
| (lower section) | | | |
| Spleen | 0.30 | 0.14 | 2.90 |
| Liver | 0.67 | 1.03 | 0.60 |
| Bone Marrow | 0.11 | | 1.00 |

These results show that labelled stem cells have crossed the blood brain barrier and migrated to the brain, brain stem, medulla oblongata as well as upper and lower sections of the spinal cord. Cells of the cell product may provide for tissue regeneration of plaques and additionally for preventing T-cells, B-cells and other cells of both the adaptive and the innate immune system to cross the blood barrier and infiltrate the nervous tissue beyond it.

The appearance of transferred stem cells in the spleen may result in therapeutic immune modulation during the course of the EAE disease.

The appearance of transferred cells in the liver may protect and regenerate the liver of EAE mice.

It is known that MS patients suffer from an enzyme deficiency required for clearance of oxygen radicals from the body. The enzyme defect and the resulting accumulation of toxins in MS patients is proportional to loss of movement control. The poisoning causes damages in the central nervous system to nerve fibers.

The homing of labelled transferred cells in the bone marrow and their release is observerd—as described in the Examples of the other model diseases in mice above—also with EAE mice.

Conclusions for Examples with Mice (Section A):

The test results show that the therapeutic cell population is effective in treating animal models of rheumatoid arthritis (RA), diabetes mellitus Type 1 (DB1), diabetes mellitus Type 2 (DB2), ischemic stroke (IS), myocardial infarction (MI) and multiple sclerosis (MS).

Without wanting to be bound by any theory it is assumed that the migration of transferred cells throughout the body correlates to their therapeutic effect of first blocking in particular T-lymphocytes in order to prevent or reduce further (auto) immune reaction, that it then causes regeneration of damaged nerve tissue and finally of further tissues of other affected organs which is damaged, e.g. in the liver.

### Section B: Clinical Trials in Humans

Example 8

In Example 8 an exemplary embodiment of the ex vivo method of providing the therapeutically active cell product was performed starting with donated human tissue. This embodiment of the method was applied for preliminary tests of the method with bone marrow donations from a small sample of 6 healthy individuals and subsequently during clinical trials starting with 3 human patients suffering from multiple sclerosis (MS):

The exemplary embodiment according to Example 8 has basically the same steps as in Example 1, using however, a different group of selected surface antigens which is suitable for removal of hematopoietic cells from a human tissue sample by in vitro depletion and recovery of non-hematopoietic stem and progenitor cells in the cell product.

For some embodiments of the method of providing a therapeutically active cell product from human tissue, the group of selected surface antigens comprises CD14, CD34, CD45 and a further member of the CD45 family, like CD45RA or CD45RO

The choice of CD34, CD45 and at least one further member of the CD45 antigen family as members of the group of selected surface antigens was made for removing the hematopoietic stem and progenitor cells, lymphocytes mediating the adaptive immune system, in particular early B- and T-cell precursor stem cells and the cells of the B-cell lineage, said cells expressing either CD34 or an antigen of the CD45 family or both.

A further advantage of the method is that the use of antibodies against CD34, CD45 and at least one additional member of the CD45 antigen family in the immuno-depletion reduces the risk that the cell product comprises cells which can cause cancer in a patient. Most CD34 expressing cells also co-express at least one member of the CD45 family and were therefore assumed to efficiently remove such co-expressing cells from the original population of cells.

The choice of antigens in the group of selected antigens may be adapted to the cellular composition of the original cell population and according to known correlations between the expression of cell surface antigens and cell types to achieve the selective removal of hematopoietic cells and optionally further cell types from the original cell population.

The following list indicates some of the cell surface antigens chosen for immuno-depletion of hematopoietic cells from human bone marrow by the inventors, because of their characteristic expression on the following cell types:
CD14: is expressed on hematopoietic cells such as on monocytes including macrophages and dendritic cells as well as on neutrophilic granulocytes of the innate immune system; also expressed on the surface of some cancer cells such as in myelomonocytic leukemia and histiocytic sarcoma and other forms of cancer.
CD34: is expressed on hematopoietic stem cells and hemangioblasts which can differentiate into both hematopoietic and endothelial cells and on a subset of mesenchymal stem cells, endothelial progenitor cells, endothelial cells of blood vessels but not lymphatics (except pleural lymphatics).
CD45 antigen family (formerly LCA—leucocyte common antigen): is expressed on almost all of the hematopoietic cells except for erythrocytes. In literature it was shown e.g. that CD45 expression varies among B-lineage cells depending on cell differentiation, in contrast to its stable expression on leukemic T cell and myeloid lineage cell lines (Acta Pathol Jpn. 1990 Feb;40(2):107-15).
CD45RA: is in particular expressed on naive T-cells.
CD45RO: is in particular expressed on activated T-cells and T-memory cells.
CD45R: is in particular expressed on B-cells and their precursors, on a sub-group of dendritic cells and other antigen-presenting cells.

In this exemplary embodiment of the method applied to human bone marrow the group of selected surface antigens comprises the antigens CD14, CD34, CD45 and as further family member of the CD45 surface antigen family CD45RA; CD45RO or CD45R are further particularly favored family members of the CD45 family.

Similar to Example 1 in mice, the method of providing a therapeutically active cell product according to the exemplary embodiment according to Example 8 comprises the following steps:
filtration of isolated bone marrow;
purification of isolated marrow by filtration washing and centrifugation;
labeling with biotinylated monoclonal antibodies against selected CD antigens CD14, CD45, CD45RA;
removal of antibodies excess by cells centrifugation and further dilution;
second labeling with anti-biotin and anti-CD34 antibodies (both conjugated to superparamagnetic iron dextran particles);
removal of excess unbound antibodies by centrifugation and resuspension of the cells and further dilution;
depletion of the labeled cells using e.g. a CliniMACS magnetic separation device (the negative fraction collected as the final product).

In the exemplary embodiment of the method of Example 8 the Climimax® separation technology of Miltenyi Biotec has been applied including the reagents, buffers, equipment and tubing. Corresponding Miltenyi Biotec specifications were essentially followed and general laboratory practice has been applied with respect to e.g. sterility. All antibodies used in this particular exemplary embodiment are commercially available, e.g. from Miltenyi Biotec, Diaclone and others (see e.g. http://www.antibodyresource.com/onlinecomp.html). Also, buffers, reagents and equipment for immune-magnetic depletion are commercially available e.g. from Miltenyi Biotec, CSL Behring GmbH e.g. for Human Serum Albumin and others.

In the exemplary embodiment of providing a therapeutically active cell product of Example 8 the following protocol has been followed:

Preparatory steps prior to in vitro immuno-magnetic depletion:
Prior to the separation procedure the amount of necessary reagents and consumables required for a CliniMACS Separation are checked, and the parameters of the working environment are recorded.
A tissue probe of human bone marrow (approx. 50 ml of bone marrow) was received in a sterile bag. This was termed fraction A and was kept at room temperature (20 to 25° C.)
The tissue probe was filtered through 200 microns filter and samples were taken for flow cytometry analyses, and microbiological and morphological studies. Dilution with CliniMACS PBS/EDTA/HSA buffer (phosphate buffered saline supplemented with 1 mM EDTA, pH7.2, and prior to use additionally with 0.5% (w/v) HSA (human serum albumin). The weight of dilution buffer added was twice the weight of the cellular product.
Centrifugation for 15 min., 500×g without brakes, re-suspension of the pellet in a volume of 95 ml±5 ml CliniMACS PBS/EDTA/HSA buffer which is a suitable volume of the filtered and washed cell suspension for magnetic labeling with biotinylated monoclonal antibodies.

A first step of immuno[-magnetic]-labelling:

It was performed with biotinylated monoclonal antibodies:
A mixture of three monoclonal antibodies (CD14, CD45, CD45RA, from Diaclone was prepared by addition of 0.5 ml of a 1 mg/ml stock solution of each antibody (yielding a total volume of 1.5 ml
Then, 6 ml of CliniMACS PBS/EDTA/HSA buffer were added to yield an antibody cocktail solution with a volume of 7.5 ml.
The total volume of the antibody cocktail (7.5 ml) was transferred to the preparation bag containing the above prepared 95 m1 filtered and washed cell suspension (final labeling volume: 102.5 ml).
The cell suspension was incubated with the biotinylated monoclonal antibody cocktail for the immuno-labelling at room temperature (19-25° C.) on the orbital rotator at approx. 25 rpm for 30 min. The number of cells incubated with the antibodies ranged in particular from 10⁷ to 5×10⁹ cells, more particularly from 3×10⁷ to 2×10⁹ cells 100 ml +/−10 ml incubation volume. Preferably the total number of cells did not exceed 1.5 or 1.2×10⁹ cells 100 ml +/−10 ml incubation volume.
The incubation was ended by addition of CliniMACS PBS/EDTA buffer supplemented with 0.5% (w/v) HAS up to a total volume of 600 ml followed by centrifugation for 15 min., 500×g without brakes for removal of excess biotinylated monoclonal antibodies
The cell pellet was re-suspended in a suitable volume per amount of cells. The volume may be increased compared to the volume recommended in standard procedures by a factor of 1.5 to 4, in particular by a factor 2 to 2.5 or 2 to 3. In the exemplary embodiment of Example 8 CliniMACS PBS/EDTA buffer supplemented with 0.5% (w/v) HAS was added to a final volume of 197.5 ml (±5ml), compared to the recommended volume of 90+7.5=102.5 ml in the standard Miltenyi Biotec protocol.

A second step of immuno-magnetic-labelling was performed with anti-biotin antibodies conjugated to iron dextran micro beads and anti-CD34 antibodies where the CliniMACS Anti-CD34 Reagent No 171-01 at a concentration of 30 mg/ml and the CliniMACS Anti-Biotin Reagent No 278-01 at a concentration of 30 mg/ml were used.
For this purpose, the entire volume of one vial (7.5ml) of each of the CliniMACS reagents (CliniMACS Anti-Biotin Reagent, CliniMACS Anti-CD34 Reagent No 171-01 at a concentration of 30 mg/ml and CliniMACS Anti-Biotin Reagent No 278-01 at a concentration of 30 mg/ml were added to the preparation bag and incubated for 30 min. at room temperature (19-25° C.) on the orbital rotator at approx. 25 rpm.
The incubation was ended by addition of CliniMACS PBS/EDTA buffer supplemented with 0.5% (w/v) HAS up to a total volume of 600 ml followed by centrifugation for 15 min., 500×g without brakes for removal of excess biotinylated monoclonal antibodies.
The sample was prepared for the magnetic separation procedure using the CliniMACS instrument by addition of the CliniMACS PBS/EDTA buffer supplemented with 0.5% (w/v) HSA to a final volume of about 150 ml and labelled as fraction B.
Samples for flow cytometry, microbiological and morphology studies were collected.
The separation procedure using the CliniMACS instrument was performed using the CD34 selection program and the separation CliniMACS Tubing Set. After completion of the procedure, the weight of each obtained fraction, the negative fraction C, the positive fraction D, and the wash fraction was calculated and samples for flow cytometry, microbiological and morphological studies were taken.
The obtained cell product (fraction C) of patients participating the clinical study was labeled with bar-coded patient information and released for transfer to the patient.

This exemplary embodiment of the in vitro depletion method according to the invention was performed ex vivo with bone marrow probes from six healthy donors resulting in original suspensions or fractions A. The obtained therapeutically active cell products or fractions C were collected after the in vitro depletion of hematopoietic cells by an immuno-depletion procedure using antibodies against the CD14 and CD34 surface antigens and at least two members of the CD45 surface antigen family, in particular CD45 and CD45RA, for the removal of the undesired hematopoietic cells. The results are shown in Table 8.1. In particular the percentage-ratio of portions of positive cells expressing particular cell surface antigens in the cell product to the portions of positives in the original population (C/A×100%) is shown and furthermore the percentage portion of positives in the total number of cells of fraction C.

The values indicated in Table 8.1 below for the percentage ratio C/A represent the median value of the number of cells expressing a particular surface antigen in fraction C divided by the median value of the number of cells expressing the surface antigen in fraction C.

TABLE 8.1

| | | | | | %-Ratio of Portions of | %-Portion of |
|---|---|---|---|---|---|---|
| Surface | Desired non-hematopoietic | | | Undesired | Positives in Cell | Positives |
| Antigen | stem cell types | | | cell types | Product vs Original | among the |
| (CD) | Pluripotent | Multipotent | Progenitor | Hematopoietic | Population (C/A) | total number of cells |
| SSEA-4 | ● | | | | 44.02 | 1.12 |
| 90 | ● | ● | ● | | 25.31 | 0.51 |
| 133 | ● | ● | ● | ▾ | 21.38 | 0.25 |
| 34/133 | ● | ● | ● | ▾ | 33.89 | 0.09 |
| 44 | | ● | | | 13.33 | 31.48 |
| 71 | | ● | | | 40.69 | 13.84 |
| 73 | | ● | | | 7.05 | 0.25 |
| 105 | | ● | ● | | 33.56 | 0.64 |
| 106 | | ● | | | 5.66 | 0.20 |
| 117 | | ● | ● | ▾ | 3.97 | 0.09 |
| 146 | | ● | ● | | 38.88 | 0.15 |
| 166 | | ● | | | 24.09 | 0.18 |
| 34 | ● | ● | ● | ▾ | 5.05 | 0.5 |
| 14 | | | | ▾ | 11.84 | 0.59 |
| 19 | | | | ▾ | 8.89 | 0.79 |
| ICAM-1 | | | | ▾ | 2.39 | 0.32 |
| 34/45 | | | | ▾ | 4.73 | 0.33 |
| 73/45 | | | | ▾ | 5.35 | 0.17 |
| 45 | | | | ▾ | 10.01 | 2.13 |
| Family | | | | | | |
| 45/45RA | | | | ▾ | 1.33 | 0.34 |
| 45RA | | | | ▾ | 1.31 | 0.38 |
| 45RO | | | | ▾ | 3.18 | 1.74 |
| 133/45 | | | | ▾ | 32.42 | 0.22 |

For example in some embodiments of the cell product obtainable by in vitro depletion of hematopoietic cells from a tissue probe according to the second aspect of the invention and in particular in some embodiments obtainable according to the method of the invention, the portions of cells expressing one or more surface antigen indicative of pluripotent stem cells such as cells expressing SSEA-4 or CD90 or CD133 or cells co-expressing CD34/CD133 amount to at least 0.01% to 1% of the total cell number, in particular of at least 0.03% or to at least 0.1% or at least 0.3% or at least 1% as measured by cytometric analysis.

In Table 8.2 below, the viability of the cell population in the various cell suspensions along the in vitro procedure are indicated as percentage of viable cells among the total number of cells in the population: Fraction A is the suspension of the original cell population of the bone marrow tissue probe, fraction B is the cell suspension after two labelling steps with antibodies and prior to immune-magnetic separation, fraction C is the cells suspension, which flowed through the column, i.e. the desired cell product depleted of hematopoietic cells and fraction D comprises the portion of hematopoietic cells which was removed from the original population of cells by being retained in the immune-magnetic column and subsequently eluted. The portion of viable cells in each fraction was determined by viability analysis based on flow cytometry measurements on a sample of cells removed from each fraction and stained with propidium iodide (PI) (Carlo Riccardi, Ildo Nicoletti (2006): “Analysis of apoptosis by propidium iodide staining and flow cytometry”, Nature Protocols 1, 1458-1461).

Table 8.2 shows the results of the viability analysis of fractions of both healthy subjects (KB4) and patients with multiple sclerosis (KB12).

TABLE 8.2
Vitality - Percentage of viable cells of
the total number of cells in the fraction

Fraction

| Subject/Patient | A | B | C | D |
|---|---|---|---|---|
| KB4 13-07 | 94.60% | 99.00% | 99.00% | 96.00% |
| KB4 13-08 | 96.00% | 98.00% | 94.00% | 75.00% |
| KB4 13-11 | 98.50% | 99.00% | 100.00% | 96.00% |
| KB4 13-13 | 97.00% | 96.00% | 95.00% | 100.00% |
| KB4 13-15 | 97.86% | 93.21% | 84.15% | 93.31% |
| KB4 13-16 | 71.25% | 83.15% | 98.34% | 98.20% |
| KB12 10-01 | 97.00% | 98.00% | 100.00% | — |
| KB12 10-008 | 89.00% | 98.00% | 96.00% | 87.00% |
| KB12 10-011 | 92.00% | 98.00% | 98.00% | 90.00% |

### Example 9

### Clinical Data with MS Patients

Patients had been administered autologous cell product obtained as described in Example 8 at t=0. The amount of cell product administered was
KB12 10-01 3,12×10e6 cells/kg body weight (95kg)
KB12 10-008 1×10e6 cells/kg b.w. (70kg)
KB12 10-011 7,3×10e6 cells/kg b.w. (56kg)

The Expanded Disability Status Scale (EDSS) as developed by John F. Kurtzke has been used for assessing clinical symptoms and quantifying the disability of the three tested multiple sclerosis patients. The results of the assessment of these patients at the time point of the transfer of the autologous stem cell product obtained according to Example 8 (Tr) and at 3, 6, 9, 12, 18 and 24 months after transfer of cell product are listed in Table 9.1:

| | Tr | 3 | 6 | 9 | 12 | 18 | 24 |
|---|---|---|---|---|---|---|---|
| KB12 10-01 | 2.50 | 2.00 | 2.50 | 2.50 | 2.50 | 2.50 | 1.5 |
| KB12 10-008 | 4.00 | 3.50 | 2.50 | 2.00 | 2.50 | 3.50 | 4.00 |
| KB12 10-011 | 4.50 | 4.00 | 2.00 | 2.00 | 1.00 | 2.00 | 2.50 |

The results in Table 9 demonstrate, that all three patients profited from the therapeutic treatment. The clinical symptoms of patient KB12 10-01 which are comparatively less severe remained constant for most of the time. This corresponds to a beneficial effect of the treatment showing that progression of the disease could be stopped and no relapse i.e. further attacks increasing the severity of symptoms occurred during the observation period.

For the patients KB12 10-008 and KB12 10-011 with more severe symptoms an improvement of their condition was found for the first 9 or 18 months of the observation period, respectively, after which time increasing symptoms were observed. For the patient KB12 10-008 the increase of MS symptoms was the largest. However, relapses which are typical for the MS disease were not observed. It is noted that after some time a second infusion would potentially be advisable.

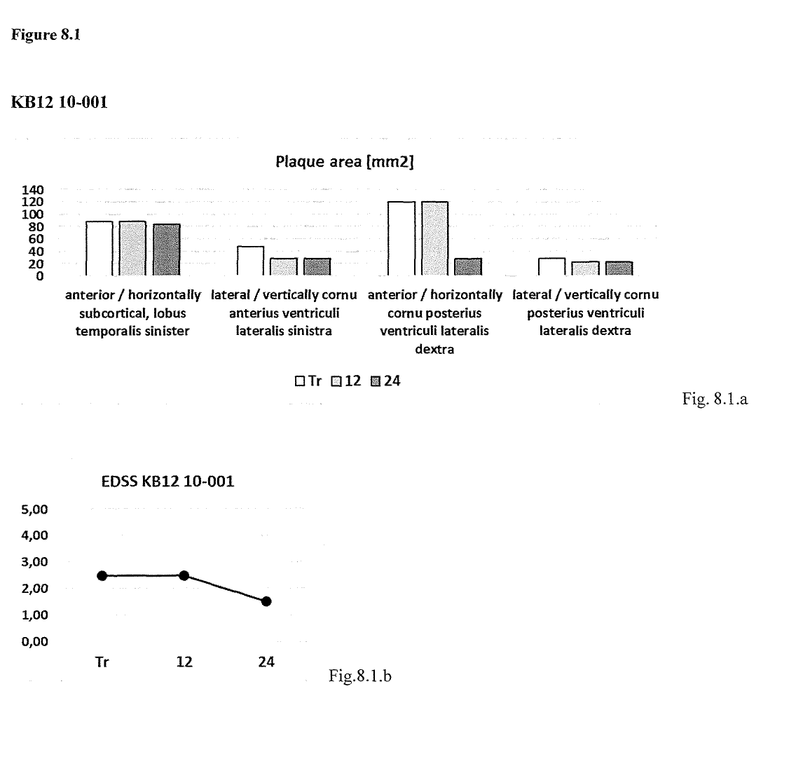

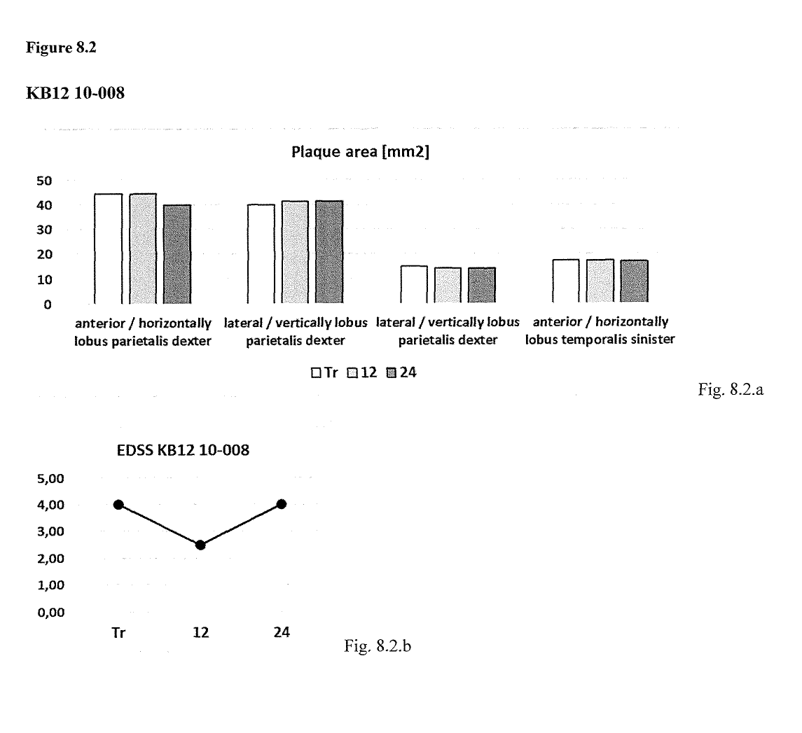

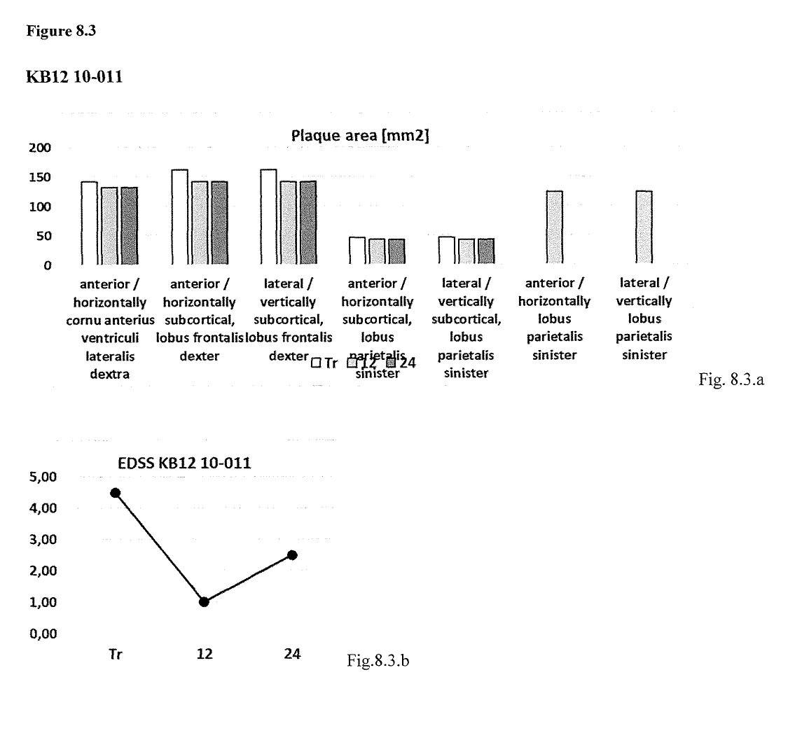

For each MS patient the most characteristic of the homogeneous plaques were selected to be analyzed by MRI imaging (see FIGS. 8.1 to 8.3). The plaque surface was measured in the largest vertical and horizontal axis area in two planes of CNS MRI imaging: anterior and lateral axis. The surface areas of the plaques were measured at three time points: at or shortly prior to (Tr) as well as 12 and 24 months after i.v. administration of the cell product as prepared according to example 8.

An exact location in the central nervous system CNS has been indicated for each lesion (plaques) of the three patients KB12 10-01, KB12 10-008, KB12 10-011 with MS analyzed. The plaques were framed and their perimeter was entered into a rectangle adjoining the longest transverse and longitudinal dimensions. On this basis the surface area was calculated.

FIGS. 8.1.a, 8.2.a and 8.3.a show for each patient KB12 10-01, KB12 10-008, KB12 10-011, respectively, at three time points of shortly prior to administration (transfer) of the cell product to the patient (Tr) as well as 12 and 24 months thereafter the change in the size of the selected characteristic plaques.

FIGS. 8.1.b, 8.2.b and 8.3.b show for each patient KB12 10-01, KB12 10-008, KB12 10-011, respectively, the EDSS score as listed in Table 9.3 (see above) of the corresponding time points.

As can be seen from the juxtapositions of the FIGS. 8.1/2/3.a and 8.1/2/3.b, there is a clear correlation between the data analysis from MRI imaging with the EDSS score for each of the three MS patients.

For more information regarding the assessment of MS see also Haber A, LaRocca N G. eds. Minimal Record of Disability for multiple sclerosis. New York: National Multiple Sclerosis Society; 1985.

Furthermore, the above mentioned MS patients were assessed according to the multiple sclerosis functional composite (MSFC) test. For references see e.g.
1. Fischer J. S., Jak A. J., Kniker J. E., Rudick R. A Multiple Sclerosis Functional Composite (MSFC) Administration and scoring manual. National Multiple Sclerosis Society. October 2001.
2. Miller D. M., Rudick R. A., Cutter G., Baier M., Fischer J. S. Clinical significance of the Multiple Sclerosis Functional Composite. Relationship to patient-reported quality of life. Arch. Neurol., 2000, 57: 1319-1324.

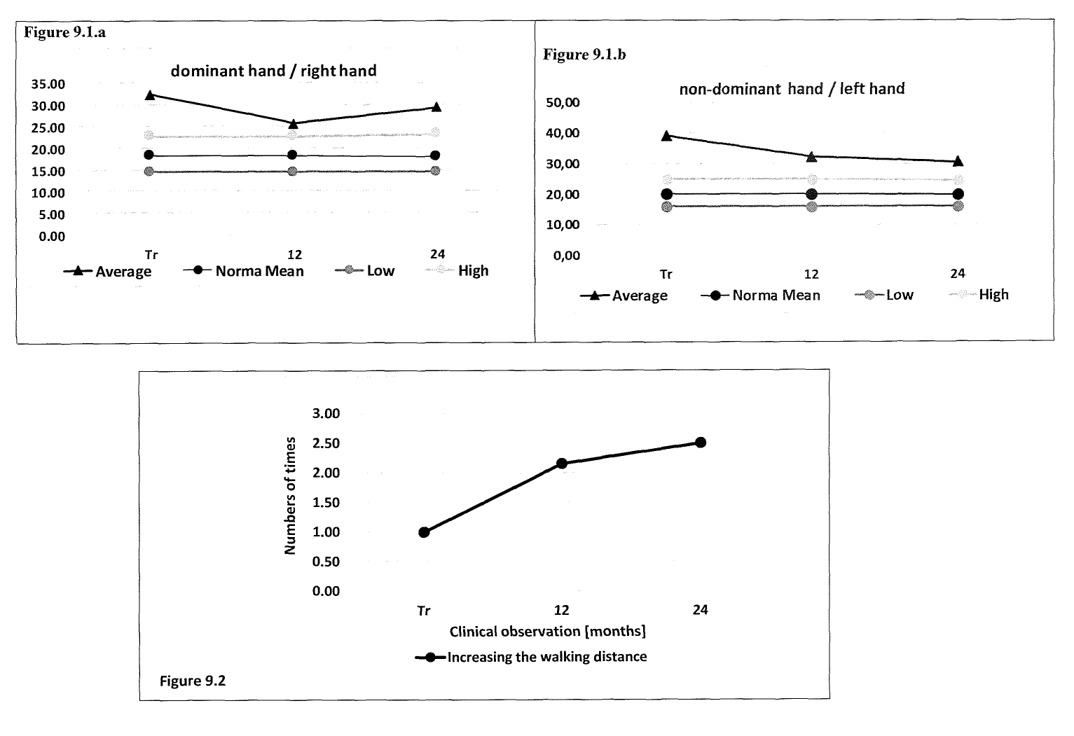

The MSFC tests were administered at the time of the transfer of the cell product (Tr) or shortly prior to it and at every control visit at 3, 6, 7, 8, 9, 10, 11, 12, 18 and 24 months thereafter. FIG. 9.1 shows the results of the 9-hole peg test (9-HPT) of the MSFC which is a test for the function of the upper extremities. FIG. 9.2 shows the result of a test measuring the ability of long distance walking without rest (as an alternative to the timed 25-foot walk of the MSFC). In FIGS. 9.1 and 9.2 average values of the three MS patients KB12 10-01, KB12 10-008, KB12 10-011 at each of the above mentioned time points are displayed.

Regarding FIG. 9.1: The 9-HPT test is a quantitative measure of the upper extremity function and it was performed according to a standard protocol (Jill S. Fischer S. J. et al., “Multiple Sclerosis Functional Composite (MFSC). Administration and Scoring Manual”, Revised October 2001). Both the dominant (FIG. 9.1.a) and non-dominant (FIG. 9.1.b) hands were tested in two consecutive trials of the dominant hand, followed immediately by two consecutive trials of the non-dominant hand. Care was taken to administer the 9-HPT on a solid table (not a rolling hospital bedside table) and to anchor the 9-HPT apparatus. In FIG. 9.1 the time required for required for filling the holes with the pegs and removing them again is indicated on the y-axis and the time points of the measurement at or shortly before the transfer of the cell product (Tr) and 12 and 24 months thereafter are indicated on the x-axis. The average values of the three MS patients tested are indicated by filled triangles and for comparison the mean, low and high amounts of time required by healthy control individuals are indicated by the circles filled in variable shades of grey to black. The results clearly reveal that the MS patients require significantly longer times than the healthy individuals prior to treatment and that at the 12 months-time point after transfer their performance in the 9-HPT test is improved significantly for the dominant and the non-dominant hand, where at the 24 months-time point the effect in the non-dominant hand is still slightly further improved but in the dominant hand it is no longer as strong. This result is in line with the observation that in response to a medical treatment of MS patients generally the performance in the 9-HPT performance is minimal with respect to the better trained dominant hand. Furthermore, these results correlate well with the results of the MRI analysis where the most beneficial therapeutic effects were observed in the right hemisphere which is responsible for the non-dominant left hands of the three right-handed MS patients presented here.

For measuring the function of the lower extremities a further test of the MSFC, namely the “Timed 25-Foot Walk”-test was administered. In this test the patient is directed to walk as quickly as possible, but safely to one end of a clearly marked 25-foot course and back. Patients may use appropriate assistive devices such as a walking stick. However, the three MS patients KB12 10-01, KB12 10-008, KB12 10-011 who scored between 2.5 and 4.5 on the EDSS scale at the time of transfer (see Table 9.1) during the clinical observation period started to run or walk abnormally fast in the “Timed 25 foot Walk”-test. Evidently, this test was too easy to properly measure actual increases in the functional performance of these patients' lower extremities after the transfer of the cell product. Therefore, the function of the lower extremities was evaluated by a physician during clinical observation inter alfa by interviewing the patients about their ability to walk over long distances during normal daily activities and scored according to the EDMUS grading scale (EGS/DSS) (Amato MP, et al. for the Evaluation of the EDMUS system (EVALUED) Study Group: European validation of a standardized clinical description of multiple sclerosis. J. Neurol. 2004; 251: 1472-1480). Furthermore, the ability to walk a distance without fatigue and was measured on a refined scale by dividing the distance walked into the following categories:
100-200 meters
200-300 meters;
300-500 meters;
over 500 meters, but still a limited distance, which was specified by the patient
over 500 meters, i.e. an unlimited distance like a healthy individual.

During the clinical observation the potential use of a walking support such as a walking stick was noted. In fact only patient KB12 10-011 needed a walking stick at the time of the transfer of cells, but three months after the transfer of the cell product he no longer needed it.

The results in FIG. 9.2 show the average increase in walking distance of the three MS patients KB12 10-01, KB12 10-008, KB12 10-011 at 12 and 24 months after transfer of the cell product. The average distance achieved at 24 months, the end of the clinical observation was 2.5 times larger than at the time of the transfer.

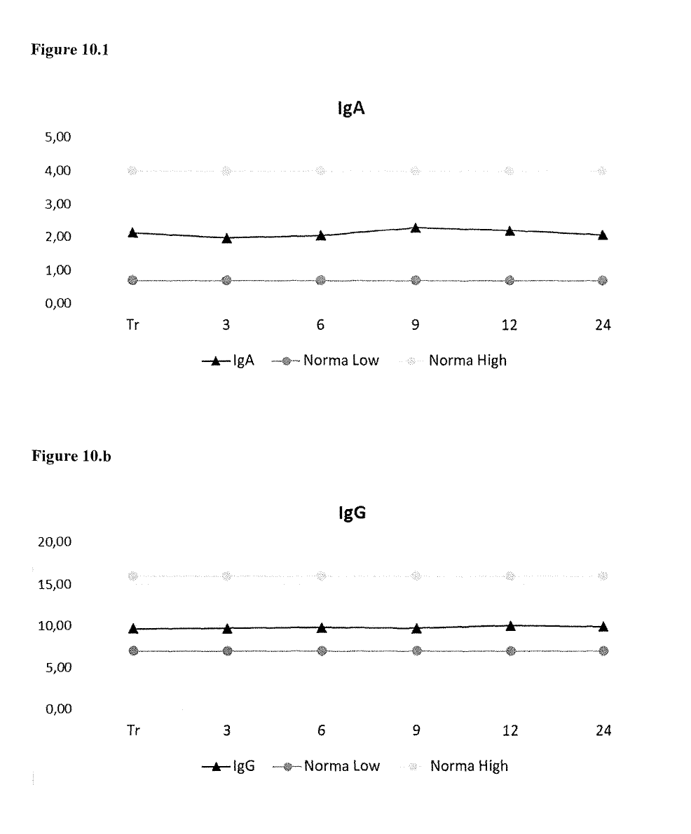

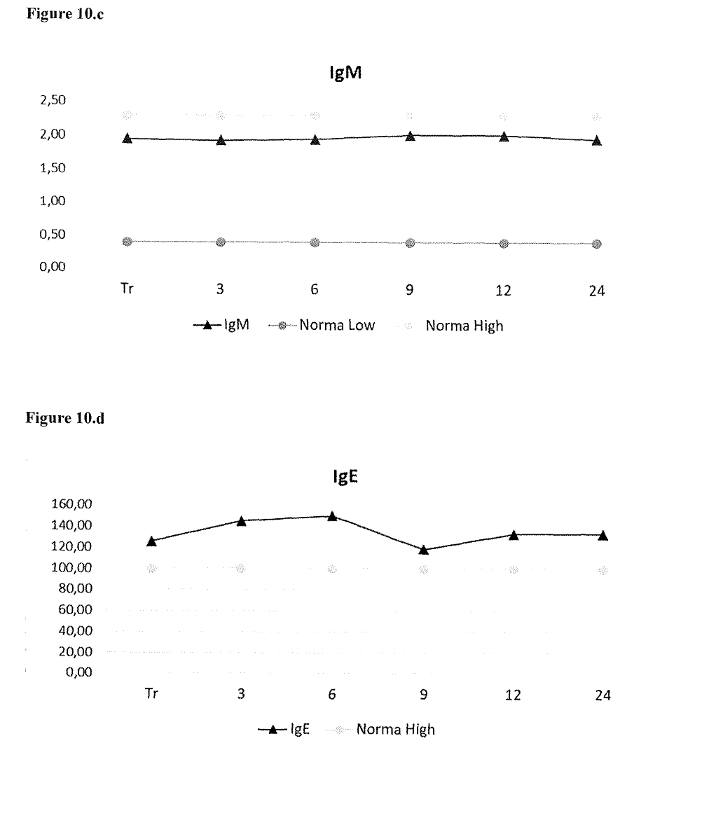

Regarding FIG. 10: The average values of the blood levels of the three MS patients of immunoglobulins

IgA, IgG, IgM and IgE were measured at the time points of the transfer of the cell product (Tr) and 12 and 24 months thereafter as indicated by filled triangles. For comparison normal low and normal high levels measured of the norm in healthy control individuals are indicated by circles filled in dark and light grey, respectively.

As shown in FIGS. 10.1, 10.2 and 10.3, the i.v. transfer of the cell product prepared according Example 8 did not result in a humoral immune response as exhibited by the blood levels of IgA (g/l), IgG (g/l), and IgM (g/l) which are all within the range of normal low and normal high blood levels of healthy individuals.

In contrast, in FIG. 10.4 the average concentration of IgE (IU/ml) of the three MS patient is above the level of healthy individuals.

1-35. (canceled)

36. A method of providing a therapeutically active cell product, the method comprising: an in vitro immuno-depletion of hematopoietic stem cells and cells of hematopoietic lineage,
wherein the therapeutically active cell product is prepared ex vivo from a tissue donation in the form of a suspension of an original population of cells,
wherein the immuno-depletion comprises a depletion of cells expressing at least one surface antigen of a group of selected surface antigens from the suspension of the original population of cells, and the group of selected surface antigens comprises CD14, CD34, CD45, and at least one further member of the CD45 surface antigen family, and
wherein the cell product comprises a portion of non-hematopoietic stem cells comprising non-hematopoietic progenitor [stem] cells, multipotent stem cells, and pluripotent stem cells.

37. The method of claim 36, wherein the suspension of the original population of cells is washed and re-suspended as a washed and/or single cell suspension prior to the in vitro depletion.

38. The method of claim 36, wherein the immuno-depletion comprises an immune-labeling procedure labeling cells expressing one or more of the selected surface antigens with specific antibodies comprising a tag,
wherein the tag is a magnetic bead or a fluorescent tag; and
wherein the immuno-depletion comprises a separation procedure for removal of the cells labeled with antibodies comprising a tag from the suspension.

39. The method of claim 38, wherein the immuno-labeling procedure is a direct immuno-labeling procedure and the tag is conjugated to the antibodies prior to a specific binding of the antibodies to the surface antigens during the immuno-labeling procedure.

40. The method of claim 39, wherein one or more steps of the immuno-labeling procedure and/or separation procedure of the immuno-depletion is performed under limiting conditions, wherein the limiting conditions favor the depletion of cells from the original population of cells which have a higher number of selected surface-antigen-specific antibodies bound per cell compared to cells which have a lower number of such selected antibodies bound per cell,
wherein one or both of the following conditions is applied:
wherein, in the direct immuno-labeling procedure, limiting incubation conditions all for only a partial saturation of the antigenic binding sites of the selected surface antigens on the cells by the tag-conjugated antibodies, or
wherein, in the separation procedure, conditions are adjusted such that cells which are labeled with two or more tags are removed from the original cell population whereas cells comprising fewer tags remain in the original cell population.

41. The method of claim 38, wherein the immuno-labeling procedure is an indirect immuno-labeling procedure and the tag is conjugated to the antibody after the specific binding of the antibodies to the surface antigens during the immuno-labeling procedure.

42. The method of claim 36, wherein the immuno-labeling is an immuno-magnetic labeling procedure, the tag is a magnetic particle, and
wherein a magnetic separation device is used in a separation procedure for depleting the immuno-magnetically labeled cells.

43. The method of claim 36, further comprising at least one direct immuno-labeling procedure, wherein the cell suspension is incubated with at least one tag-conjugated surface antigen specific antibody, said tag-conjugated surface antigen specific antibody being conjugated to a magnetic particle or a fluorescent tag.

44. The method of claim 36, further comprising at least one indirect immuno-labeling procedure,
wherein, in a first step of the method, the cell population is incubated with one or more surface antigen specific primary antibodies,
wherein after the first step, excess of the one or more unbound primary antibodies are removed by centrifugation followed by re-suspension of the cells;
wherein, in a second step of the method, the cell population is incubated with at least one tag-conjugated secondary antibody and/or with at least one other tag-conjugated reagent that specifically binds to primary antibodies.

45. The method of claim 44, wherein one or more steps of the immuno-labeling procedure and/or separation procedure of the immuno-depletion is performed under limiting conditions, wherein the limiting conditions favor the depletion of cells from the original population of cells which have a higher number of selected surface-antigen-specific antibodies bound per cell compared to cells which have a lower number of such selected antibodies bound per cell,
wherein one or more of the following conditions is applied:
in the first step of the indirect immunolabeling procedure, limiting conditions allow for only a partial saturation of the antigenic binding sites of the selected surface antigens on the cells by the primary antibodies, or
in the second step of the indirect immuno-labeling procedure, limiting incubation conditions allow for only a partial saturation of the antigenic binding sites of the primary antibodies by the tag-conjugated secondary antibodies, or
in the first step of the indirect immuno-labeling, standard incubation conditions are applied to allow for a maximized saturation of the selected surface antigenic binding sites while minimizing unspecific binding of the primary antibodies to the cells and in the second step of the indirect immuno-labeling limiting incubation conditions allow for only a partial saturation of the antigenic binding sites on the primary antibodies by the tag-conjugated secondary antibodies, or
in the first step of the indirect immuno-labeling limiting incubation conditions are adjusted to allow for only a partial saturation of the selected surface antigenic binding sites on the cells, and in the second step of the indirect immuno-labeling standard incubation conditions allow for a maximized saturation of the antigenic binding sites on the primary antibodies; or
wherein, in the separation procedure, conditions are adjusted such that cells which are labeled with two or more tags are removed from the original cell population whereas cells comprising fewer tags remain in the original cell population.

46. The method of claim 44,
wherein, in the first step of the immuno-labeling, the cell population is incubated with primary antibodies against at least 3 different selected surface antigens at the same time, and each of the primary antibodies is incubated according to standard incubation conditions.

47. The method of claim 36, wherein incubation conditions of one or more steps of the immuno-depletion are adjusted to allow for only a partial saturation of antigenic binding sites of at least one of the selected surface antigens, said selected surface antigens comprising at least one of CD14, CD19, CD34, another member of the CD45 surface antigen family, CD117, and ICAM-1.

48. The method of claim 47, wherein the partial saturation of the antigenic binding sites is achieved by reducing the contact efficiency between antibody and antigen or by reducing the binding probability or by reducing the binding strength between the antibody and antigen.

49. The method of claim 36, wherein the in vitro immuno-depletion comprises an immuno-labeling procedure for labeling the cells expressing at least one surface antigen of the group of selected surface antigens,
wherein the immuno-labeling is performed in at least two stages,
wherein in a first stage cells are labeled with antibodies against selected surface antigens except for antibodies against CD34, and
wherein in a second stage performed after the first stage, cells are labeled with antibodies against CD34.

50. The method of claim 36, wherein the duration of the ex vivo manipulations comprising in vivo depletion is limited to less than 10 hours.

51. The method of claim 36, wherein the group of selected surface antigens additionally comprises at least one surface antigen which is:
characteristic for one or more cell types of hematopoietic stem cells or cells of hematopoietic lineage, including CD2, CD3, CD 10, CD11b, CD15 (SSEA-1), CD16, CD19, CD44, CD56, CD123, CD235a, CD49f, ICAM-1,
absent or essentially absent on mesenchymal stem cells or on cells which have been reported to promote tissue regeneration, including CD11a/LFA-1, CD31, CD80, CD86, CD40, and CD144, or
characteristic for tumor cells or cells prone to transformation into tumor cells, including CD9, CD15, CD20, CD24, CD31, CD38, CD44, CD117, CD146, CD166, CD171, CD184, CD324, CD325, CD326, CD338, ERb2, and HER2/neu.

52. The method of claim 36, wherein the at least one further member of the CD45 family is selected from CD45R, CD45RA, CD45RB, CD45RC, CD45RAB, CD45RAC, CD45RBC, CD45RO, or CD45R(ABC).

53. The method of claim 36, wherein the group of selected surface antigens comprises CD45RA or CD45RO.

54. The method of claim 36, wherein the therapeutically active cell product is administered to a subject.

55. The method of claim 54, wherein the therapeutically active cell product is autologous to the subject.

56. The method of claim 54, wherein the subject has a disease, disorder, or condition selected from the group consisting of: lost or damaged tissue, neurological disease, autoimmune disease, multiple sclerosis, diabetes mellitus type I, diabetes mellitus type II, rheumatoid arthritis, myocardial infarction, and ischemic stroke.

57. A therapeutically active cell product produced by the method of claim 36.

58. A pharmaceutical formulation comprising the therapeutically active cell product produced by the method of claim 36.

59. A method for regenerating tissue in a subject in need thereof, comprising administering a therapeutically effective amount of the therapeutically active cell product produced by the method of claim 36.

60. A therapeutically active cell product comprising non-hematopoietic progenitor [stem] cells, multipotent stem cells and pluripotent stem cells obtainable ex vivo from an original population of cells derived from a tissue probe, by in vitro depletion of hematopoietic cells, wherein the portion of cells in the cell product expressing at least one surface antigen of a group of surface antigens characteristic of hematopoietic is reduced by a factor of at least 2, or at least 3, or least 5, least 10, or at least 50 compared to the original population of cells, and wherein the group of surface antigens characteristic of hematopoietic surface antigens comprises at least two members of the CD45 surface antigen family.

61. A method for regenerating tissue in a subject in need thereof, comprising:
obtaining a tissue sample from the subject;
subjecting the tissue sample to an in vitro immuno-depletion of hematopoietic stem cells and cells of hematopoietic lineage, wherein the immuno-depletion comprises a depletion of cells expressing at least one surface antigen of a group of selected surface antigens from the suspension of original population of cells, and the group of selected surface antigens comprises CD14, CD34, CD45, and at least one further member of the CD45 surface antigen family; and
administering the product of the immuno-depletion to the subject.
